SEQUENCE LISTING (2 sequences)

SEQ ID NO: 1 - DNA, 21 bp, Artificial
  Synthetic Primers
aagcttatggatggatggaga

SEQ ID NO: 2 - DNA, 21 bp, Artificial
  Synthetic Primers
ggatcctcagcgggccaaggc

(19) United States
(12) Patent Application Publication
(10) Pub. No.: US 2010/0210635 A1
(43) Pub. Date: Aug. 19, 2010

(54) RENIN INHIBITORS

(75) Inventors: Benjamin Jones, Cardiff-By-The-Sea, CA (US); Andre A. Kiryanov, San Diego, CA (US); Stephen W. Kaldor, Del Mar, CA (US)

Correspondence Address: TAKEDA SAN DIEGO, INC., 10410 SCIENCE CENTER DRIVE, SAN DIEGO, CA, 92121, US

(73) Assignee: Takeda Pharmaceutical Company Limited, Osaka (JP)

(21) Appl. No.: 12/294,906

(22) PCT Filed: Mar. 23, 2007
(86) PCT No.: PCT/US07/64814

§ 371 Date: Nov. 7, 2008

Related U.S. Application Data
(60) Provisional application No. 60/787,564, filed on Mar. 29, 2006.

(51) Int. Cl. A61K 31/538; C07D 241/04; A61K 31/495; C12N 9/99; C07D 403/06; A61K 31/496; C07D 405/06; C07D 401/10; C07D 265/36; C07D 413/02; C07D 403/02; A61K 31/5377; A61P 9/00; A61P 9/12; A61P 29/00; A61P 35/00; A61P 25/00; A61P 13/12

(52) U.S. Cl. 514/230.5; 544/391; 514/255.01; 435/184; 544/383; 514/252.12; 544/373; 514/254.08; 544/379; 514/254.1; 544/360; 514/253.12; 544/105; 544/369; 514/254.02; 544/357; 514/252.11; 544/121; 514/235.8

(57) ABSTRACT

Compounds, pharmaceutical compositions, kits and methods are provided for use with Renin that comprise a compound selected from the group consisting of: wherein the variables are as defined herein.

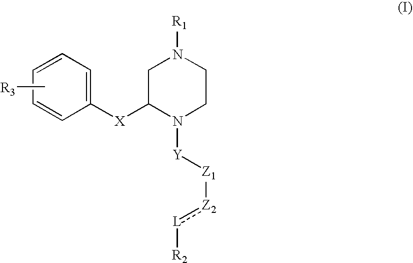

### FIELD OF THE INVENTION

The present invention relates to compounds that may be used to inhibit Renin, as well as compositions of matter and kits comprising these compounds. The invention also relates to methods for inhibiting Renin and treatment methods using compounds according to the present invention.

### BACKGROUND OF THE INVENTION

The renin-angiotensin-aldosterone system (“RAAS”) is one of the hormonal mechanisms involved in regulating pressure/volume homeostasis and also in the development of hypertension, a condition that can progress to more serious cardiovascular diseases such as congestive heart failure. Activation of RAAS begins with secretion of the enzyme Renin from juxtaglomerular cells in the kidney.

Renin, a member of the aspartyl protease family, passes from the kidneys into the blood where it cleaves angiotensinogen to generate the decapeptide angiotensin I. Angiotensin I is then cleaved in the lungs, the kidneys and other organs by the angiotensin-converting enzyme (ACE) to form the octapeptide angiotensin II. Angiotensin II, which is known to work on at least two receptor subtypes (AT₁ and AT₂), increases blood pressure both directly by arterial vasoconstriction and indirectly by liberating from the adrenal glands the sodium-ion-retaining hormone aldosterone. Angiotensin II also produces other physiological effects such as promoting sodium and fluid retention, inhibiting Renin secretion, increasing sympathetic nervous system activity, stimulating vasopressin secretion, causing a positive cardiac inotropic effect and modulating other hormonal systems.

Modulation of the RAAS represents a major advance in the treatment of cardiovascular diseases. In particular, the rationale to develop Renin inhibitors lies in its specificity (Kleinert H. D., Cardiovasc. Drugs, 1995, 9, 645). The only substrate known for Renin is angiotensinogen, which can only be processed (under physiological conditions) by Renin. Inhibitors of the enzymatic activity of Renin are therefore expected to bring about a reduction in the formation of angiotensin I and angiotensin II.

In view of the foregoing, Renin is an especially attractive target for the discovery of new therapeutics for cardiovascular disease, hypertension, congestive heart failure, myocardial infarction, renal protection, inflammation, neurological diseases, cancer and other diseases. Accordingly, there is a need to find new Renin inhibitors for use as therapeutic agents to treat human diseases. In particular, there is a continued need for metabolically stable, orally bioavailable Renin inhibitors that can be prepared on a large scale.

### SUMMARY OF THE INVENTION

The present invention relates to compounds that have activity for inhibiting Renin. The present invention provides compounds, pharmaceutical compositions, articles of manufacture and kits comprising these compounds, and also methods of using and method of preparing these compounds.

In one aspect, a pharmaceutical composition is provided that comprises a Renin inhibitor according to the present invention as an active ingredient. Pharmaceutical compositions according to the invention may optionally comprise 0.001%-100% of one or more inhibitors of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

In another aspect, the invention provides kits and other articles of manufacture for treating disease states associated with Renin. In one embodiment, a kit is provided that comprises a composition comprising at least one Renin inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another aspect, the invention provides an article of manufacture that comprises a composition comprising at least one Renin inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

In another aspect, the invention provides methods for preparing compounds, compositions and kits according to the present invention. For example, several synthetic schemes are provided herein for synthesizing compounds according to the present invention.

In another aspect, the invention provides methods for using compounds, compositions, kits and articles of manufacture according to the present invention.

In one embodiment, the compounds, compositions, kits and articles of manufacture are used to inhibit Renin.

In another embodiment, the compounds, compositions, kits and articles of manufacture are used to treat a disease state for which Renin possess activity that contributes to the pathology and/or symptomology of the disease state.

In another embodiment, a compound is administered to a subject wherein Renin activity within the subject is altered, preferably reduced.

In another embodiment, a prodrug of a compound is administered to a subject that is converted to the compound in vivo where it inhibits Renin.

In another embodiment, a method of inhibiting Renin is provided that comprises contacting a Renin with a compound according to the present invention.

In another embodiment, a method of inhibiting Renin is provided that comprises causing a compound according to the present invention to be present in a subject in order to inhibit Renin in vivo.

In another embodiment, a method of inhibiting a Renin is provided that comprises administering a first compound to a subject that is converted in vivo to a second compound wherein the second compound inhibits Renin in vivo. It is noted that the compounds of the present invention may be the first or second compounds.

In another embodiment, a therapeutic method is provided that comprises administering a compound according to the present invention.

In another embodiment, a method of treating a condition in a patient that is known to be mediated by Renin, or which is known to be treated by Renin inhibitors, comprising administering to the patient a therapeutically effective amount of a compound according to the present invention.

In another embodiment, a method is provided for treating a disease state for which Renin possess activity that contributes to the pathology and/or symptomology of the disease state, the method comprising: causing a compound according to the present invention to be present in a subject in a therapeutically effective amount for the disease state.

In another embodiment, a method is provided for treating a disease state for which Renin possess activity that contributes to the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which Renin possess activity that contributes to the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by Renin, or that is known to be treated by Renin inhibitors.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well know in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to inhibit Renin and thus may themselves be prodrugs for another compound. Such prodrugs of another compound may or may not themselves independently have Renin inhibitory activity.

### BRIEF DESCRIPTION OF THE FIGURES

FIG. 1 illustrates SEQ ID NO: 1 and SEQ ID NO: 2 referred to in this application.

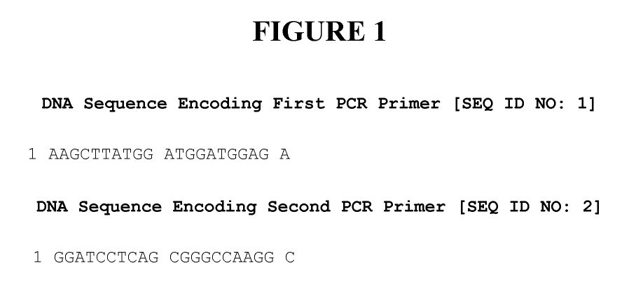

### DEFINITIONS

Unless otherwise stated, the following terms used in the specification and claims shall have the following meanings for the purposes of this Application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃₋₈ rings such as cyclopropane, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkoxy” means the radical —O-alkyl; the alkyl group is as defined in this and can be optionally substituted.

“Alkyl” represented by itself means a straight or branched, saturated or unsaturated, aliphatic radical having a chain of carbon atoms, optionally with oxygen (See “oxaalkyl”) or nitrogen atoms (See “azaalkyl”) between the carbon atoms. C_{X} alkyl and C_{X-Y} alkyl are typically used where X and Y indicate the number of carbon atoms in the chain. For example, C₁₋₆ alkyl includes alkyls that have a chain of between 1 and 6 carbons (e.g., methyl, ethyl, propyl, isopropyl, butyl, sec-butyl, isobutyl, tert-butyl, pentyl, hexyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenylethyl, 1-phenylethyl, 3-phenylpropyl, 2-thienylmethyl, 2-pyridinylmethyl and the like).

“Alkenyl” means a straight or branched, carbon chain that contains at least one carbon-carbon double bond. Examples of alkenyl include vinyl, allyl, isopropenyl, pentenyl, hexenyl, heptenyl, 1-propenyl, 2-butenyl, 2-methyl-2-butenyl, and the like.

“Alkynyl” means a straight or branched, carbon chain that contains at least one carbon-carbon triple bond. Examples of alkynyl include ethynyl, propargyl, 3-methyl-1-pentynyl, 2-heptynyl and the like.

“Alkylene”, unless indicated otherwise, means a straight or branched, saturated or unsaturated, aliphatic, divalent radical. C_{X} alkylene and C_{X-Y} alkylene are typically used where X and Y indicate the number of carbon atoms in the chain. For example, C₁₋₆ alkylene includes methylene (—CH₂—), ethylene (—CH₂CH₂—), trimethylene (—CH₂CH₂CH₂—), tetramethylene (—CH₂CH₂CH₂CH₂—), 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like.

“Alkenylene” means a straight or branched, divalent carbon chain having one or more carbon-carbon double bonds. Examples of alkenylene include ethene-1,2-diyl, propene-1,3-diyl, methylene-1,1-diyl, and the like.

“Alkynylene” means a straight or branched, divalent carbon chain having one or more carbon-carbon triple bonds. Examples of alkynylene include ethyne-1,2-diyl, propyne-1,3-diyl, and the like.

“Alkylidene” means a straight or branched saturated or unsaturated, aliphatic radical connected to the parent molecule by a double bond. C_{X} alkylidene and C_{X-Y} alkylidene are typically used where X and Y indicate the number of carbon atoms in the chain. For example, C₁₋₆ alkylidene includes methylidene (═CH₂), ethylidene (═CHCH₃), isopropylidene (═C(CH₃)₂), propylidene (═CHCH₂CH₃), allylidene (═CH—CH═CH₂), and the like).

“Amino” means the radical —NRₐR_{b}, where Rₐ and R_{b} are each independently hydrogen or a non-hydrogen substituent. Representative amino groups include, without limits, —NH₂, —NHCH₃, —N(CH₃)₂, —NHC₁₋₁₀-alkyl, —N(C₁₋₁₀-alkyl)₂, —NHaryl, —NHheteroaryl, —N(aryl)₂, —N(heteroaryl)₂, and the like. Optionally, Rₐ and R_{b} together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (see Heteroaryl).

“Aryl” means a monocyclic or polycyclic ring assembly where all the ring atoms are carbon atoms, and at least one of the rings comprising the ring assembly is an aromatic ring. If one or more ring atoms is not carbon (e.g., N, S), the ring assembly is a heteroaryl. C_{X} aryl and C_{X-Y} aryl are typically used where X and Y indicate the number of carbon atoms in the ring.

“Azaalkyl” means an alkyl, as defined above, except where one or more substituted or unsubstituted nitrogen atoms (—N—) are positioned between carbon atoms of the alkyl. For example, an (C₂₋₆) azaalkyl refers to a chain comprising between 2 and 6 carbons and one or more nitrogen atoms positioned between the carbon atoms.

“Bicyclic” means a two-ringed ring assembly where the two rings are fused together, linked by a single bond or linked by two bridging atoms.

“Bicycloalkyl” means a saturated or partially unsaturated fused bicyclic or bridged polycyclic ring assembly.

“Bicycloaryl” means a ring assembly of two rings, wherein the rings are linked by a single bond or fused and at least one of the rings comprising the ring assembly is an aromatic ring. C_{X} bicycloaryl and C_{X-Y} bicycloaryl are typically used where X and Y indicate the number of carbon atoms in the bicyclic ring assembly and directly attached to the ring.

“Bridging ring” as used herein refers to a ring that is bonded to another ring to form a compound having a bicyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRₐR_{b} where Rₐ and R_{b} are each independently hydrogen or a non-hydrogen substituent.

“Carbocycle” means a ring consisting of carbon atoms.

“Carbocyclic ketone derivative” means a carbocyclic derivative wherein the ring contains a —C(═O)— moiety.

“Carbonyl” typically means a divalent radical —C(═O)—. It is noted that the term “carbonyl” when referring to a monovalent substituent can alternatively refer to a substituted carbonyl or acyl group, —C(═O)Rₐ, where Rₐ is hydrogen or a non-hydrogen substituent on the carbonyl carbon, forming different carbonyl-containing groups including acids, acid halides, aldehydes, amides, esters, and ketones.

“Carboxy” typically means a divalent radical —C(O)O—. It is noted that the term “carboxy” when referring to a monovalent substituent means a substituted carboxy, —C(O)ORₐ, where Rₐ is hydrogen or a non-hydrogen substituent on the carboxyl group forming different carboxy containing groups including acids and esters. It is further noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a radical comprising a non-aromatic, saturated or partially unsaturated, monocyclic, fused or bridged polycyclic ring assembly. C_{X} cycloalkyl and C_{X-Y} cycloalkyl are typically used where X and Y indicate the number of carbon atoms in the ring assembly. For example, C₃₋₁₀ cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

“Cycloalkylene” means a divalent radical comprising a saturated or partially unsaturated, monocyclic or polycyclic ring assembly. C_{X} cycloalkylene and C_{X-Y} cycloalkylene are typically used where X and Y indicate the number of carbon atoms in the ring assembly.

“Cyclyl” means a mono- or polycyclic radical, typically a mono-, bi- or tricyclic, unsaturated, partially saturated or saturated ring system with typically 3 to 22, more typically 3 to 14, most typically 3-7, ring atoms and is unsubstituted or substituted by one or more substituents independently selected typically from the substituents as defined in this Application.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a multi-ring assembly wherein the rings comprising the ring assembly are so linked that the ring atoms that are common to two rings are directly bound to each other. The fused ring assemblies may be saturated, partially saturated, carbocyclics, heterocyclics, aromatics, heteroaromatics, and the like. Non-exclusive examples of common fused rings include decalin, naphthalene, anthracene, phenanthrene, indole, benzofuran, purine, quinoline, and the like.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like (e.g., halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroalkyl” means alkyl, as defined in this Application, provided that one or more of the atoms within the alkyl chain is a heteroatom.

“Heteroaryl” means a monocyclic or polycyclic ring assembly wherein at least one ring atom is a heteroatom and the remaining ring atoms are carbon, and at least one of the rings comprising the ring assembly is an aromatic ring. Monocyclic heteroaryl groups include, but are not limited to, cyclic aromatic groups having five or six ring atoms, wherein at least one ring atom is a heteroatom and the remaining ring atoms are carbon. The nitrogen atoms of such heteroaryl rings can be optionally quaternerized and the sulfur atoms of such heteroaryl rings can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes polycyclic ring assemblies, wherein a heteroaromatic ring is fused or linked by a bond to one or more rings independently selected from the group consisting of an aromatic ring, a cycloalkyl ring, a cycloalkenyl ring, a heterocycloalkyl ring and another heteroaromatic ring. Bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The polycylic heteroaryl ring assembly can be attached to the parent molecule through either the heteroaryl group itself or the aryl, cycloalkyl, cycloalkenyl or heterocycloalkyl group to which it is fused. The heteroaryl groups of this invention can be substituted or unsubstituted.

“Heterobicycloaryl” means bicycloaryl, as defined in this Application, provided that one or more of the atoms within the ring assembly is a heteroatom. For example, hetero(C₄₋₁₂)bicycloaryl as used in this Application includes, but is not limited to, indoline, 2-amino-4-oxo-3,4-dihydropteridin-6-yl, tetrahydroisoquinolinyl, and the like.

“Heterocycloalkyl” means cycloalkyl, as defined in this Application, provided that one or more of the atoms forming the ring is a heteroatom. Non-exclusive examples of heterocycloalkyl include piperidyl, 4-morpholyl, 4-piperazinyl, pyrrolidinyl, perhydropyrrolizinyl, 1,3-dioxanyl, 1,4-dioxanyl and the like.

“Heteroatom” refers to an atom that is not a carbon atom. Particular examples of heteroatoms include, but are not limited to nitrogen, oxygen, and sulfur.

“Heteroatom moiety” includes a moiety where the atom by which the moiety is attached is not a carbon. Examples of heteroatom moieties include —N═, —NR—, —N⁺(O⁻)═, —O—, —S— or —S(O)₂—, wherein R is hydrogen or a non-hydrogen substituent.

“Heterobicycloalkyl” means bicycloalkyl, as defined in this Application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like.

“Heterocyclyl” refers to a mono- or polycyclic radical, typically a mono-, bi- or tricyclic, unsaturated, partially saturated or saturated ring system with typically 3 to 22, more typically 3 to 14, most typically 3-7, ring atoms, and with one or more, preferably one to four, heteroatoms independently selected from nitrogen, oxygen, sulfur, S(═O)—, —S(═O)₂—, and is unsubstituted or substituted by one or more substituents independently selected typically from the substituents defined in this Application.

“Heterocycloalkylene” means cycloalkylene, as defined in this Application, provided that one or more of the ring member carbon atoms is replaced by a heteroatom.

“Hydroxy” means the radical —OH.

“IC₅₀” refers to the molar concentration of an inhibitor that produces 50% inhibition of the target enzyme.

“Iminoketone derivative” means a derivative comprising the moiety —C(═NR)—, wherein R is hydrogen or a non-hydrogen substituent attached to the nitrogen.

“Isomers” mean any compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four different substituents (where no two are the same) is termed a “chiral center.” A compound with one chiral center has two enantiomeric forms of opposite chirality. A mixture of equal amounts of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Leaving group” means a moiety that can be displaced by another moiety, such as by nucleophilic attack, during a chemical reaction. Leaving groups are well known in the art and include, for example, halides and OSO₂R′ where R′ is, for example, alkyl, haloalkyl, or aryl optionally substituted by halo, alkyl, alkoxy, amino, and the like. Non-limiting examples of leaving groups include chloro, bromo, iodo, mesylate, tosylate, and other similar groups.

“Moiety” means an interconnected group of atoms, generally referred to by its most characteristic structural component. For example, a “carbonyl moiety” refers to groups that contain a carbonyl group.

“Nitro” means the radical —NO₂.

“Oxaalkyl” means an alkyl, as defined above, except where one or more oxygen atoms (—O—) are positioned between carbon atoms of the alkyl. For example, an (C₂₋₆)oxaalkyl refers to a chain comprising between 2 and 6 carbons wherein one or more oxygen atoms is positioned between two carbon atoms.

“Oxy” typically means the radical —O—. It is noted that the term “oxy” when referring to a monovalent radical can alternatively refer to a substituents oxy group, —OR—, where R is hydrogen or a non-hydrogen substituent on the oxy radical forming oxy-containing groups including hydroxy, alkoxy, aryloxy, heteroaryloxy and carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound that is convertible in vivo metabolically into an inhibitor according to the present invention. The prodrug itself may or may not also have Renin inhibitory activity. For example, an inhibitor comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, an inhibitor comprising an amine group may be administered as an amide or as an N-alkyl (particularly N-methyl or N-ethyl) that is converted by hydrolysis or oxidation in vivo to the amine compound.

“Protected derivatives” means derivatives of inhibitors in which a reactive site or sites are blocked with protecting groups. Protected derivatives are useful in the preparation of inhibitors or in themselves may be active as inhibitors. Examples of protected group includes, but are not limited to, acetyl, tetrahydropyran, methoxymethyl ether, (3-methoxyethoxymethyl ether, ρ-methoxybenzyl, methylthiomethyl ether, pivaloyl, silyl ether, carbobenzyloxy, benzyl, tert-butoxycarbonyl, ρ-methoxyphenyl, 9-fluorenylmethyloxycarbonyl, acetals, ketals, acylals, dithianes, methylesters, benzyl esters, tert-butyl esters, and silyl esters. A comprehensive list of suitable protecting groups can be found in T. W. Greene, Protecting Groups in Organic Synthesis, 3rd edition, John Wiley & Sons, Inc. 1999.

“Ring” means a carbocyclic or a heterocyclic system.

“Substituent convertible to hydrogen in vivo” means any group that is convertible to a hydrogen atom by enzymological or chemical means including, but not limited to, hydrolysis, reduction and oxidation. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl (—(O)CO—C(CH₃)₃), benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine; —C(O)CH(NH2)CH₃), Arg (arginine), Asn (asparagine), Asp (aspartic acid), Cys (cysteine), Glu (glutamic acid), His (histidine), Ile (isoleucine), Leu (leucine; —C(O)CH(NH₂)CH₂CH(CH₃)₂), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups (—(O)CO—C(CH₃)₃), and the like. Suitable peptide residues include peptide residues comprising two to five, and optionally two to three, of the aforesaid amino acid residues. Examples of such peptide residues include, but are not limited to, residues of such peptides as Ala-Ala (—C(O)CH(NH)CH₃—C(O)CH(NH₂)CH₃)), Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups (—(O)CO—C(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl). Further examples of substituents “convertible to hydrogen in vivo” include enzymatic oxidizable groups such as N-alkyls, particularly N-methyl and N-ethyl.

“Substituted or unsubstituted” or “optionally substituted” means that a given moiety may consist of only hydrogen atoms bound at available valences (unsubstituted) or may further comprise one or more non-hydrogen atoms bound through available valencies (substituted). The substituents of an “optionally substituted” group may include, without limitation, one or more substituents independently selected from the group or designated subsets thereof, aldehyde, (C₁₋₁₀)alkyl, alkylene, alkylidene, amide, amino, aminoalkyl, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted.

In one particular embodiment, examples of substituents include, but are not limited to, hydrogen, halo, nitro, cyano, thio, oxy, hydroxy, carbonyloxy, (C₁₋₁₀)alkoxy, (C₄₋₁₄)aryloxy, (C₁₋₁₃)heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, (C₁₋₁₀)haloalkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, (C₃₋₁₂)heterocycloalkyl(C₁₋₁₀)alkyl, aryl(C₁₋₁₀)alkyl, (C₁₋₁₀)heteroaryl(C₁₋₅)alkyl, (C₉₋₁₂)bicycloaryl(C₁₋₅)alkyl, (C₈₋₁₂)heterobicycloaryl(C₁₋₅)alkyl, (C₃₋₁₂)cycloalkyl, (C₃₋₁₂)heterocycloalkyl, (C₉₋₁₂)bicycloalkyl, (C₃₋₁₂)heterobicycloalkyl, (C₄₋₁₂)aryl, (C₁₋₁₀)heteroaryl, (C₉₋₁₂)bicycloaryl and (C₄₋₁₂)heterobicycloaryl, the substituents are as defined herein. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, (C₁₋₁₀)heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, (C₁₋₁₀) haloalkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, (C₃₋₁₂)heterocycloalkyl(C₁₋₁₀)alkyl, aryl(C₁₋₁₀)alkyl, (C₁₋₁₀)heteroaryl(C₁₋₅)alkyl, (C₉₋₁₂)bicycloaryl(C₁₋₁₀)alkyl, (C₈₋₁₂)heterobicycloaryl(C₁₋₅)alkyl, (C₃₋₁₂)cycloalkyl, (C₃₋₁₂)heterocycloalkyl, (C₉₋₁₂)bicycloalkyl, (C₃₋₁₂)heterobicycloalkyl, (C₄₋₁₂)aryl, (C₁₋₁₀) heteroaryl, (C₉₋₁₂)bicycloaryl and (C₄₋₁₂)heterobicycloaryl, the substituents are as defined herein.

“Sulfinyl” means the radical —S(O)—. It is noted that the term “sulfinyl” when referring to a monovalent substituent can alternatively refer to a substituted sulfinyl group, —S(═O)R, where R is hydrogen or a non-hydrogen substituent on the sulfur atom forming different sulfinyl groups including sulfinic acids, sulfinamides, sulfinyl esters, and sulfoxides.

“Sulfonyl” means the radical —S(O)₂—. It is noted that the term “sulfonyl” when referring to a monovalent substituent can alternatively refer to a substituted sulfonyl group, —S(═O)₂R, where R is hydrogen or a non-hydrogen substituent on the sulfur atom forming different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thiocarbonyl” means the radical —C(S)—. It is noted that the term thiocarbonyl when referring to a monovalent substituent can alternatively refer to a substituted thiocarbonyl group, —C(═S)₂R, where R is hydrogen or a non-hydrogen substituent on the carbon atom forming different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} may each independently be hydrogen or any other substituent where the atom attached to the carbon is not a hydrogen atom. Hence, —CF₃, —CH₂OH and —CH₂CN, for example, are all C₁ alkyls.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} may each independently be hydrogen or any other substituent where the atom attached to the carbon is a heteroatom or cyano. Hence, CF₃, CH₂OH and CH₂CN, for example, are all C₁ alkyls.

### DETAILED DESCRIPTION OF THE INVENTION

The present invention relates to compounds, compositions, kits and articles of manufacture that may be used to inhibit Renin. The present invention also relates to methods for inhibiting Renin and treatment methods using compounds according to the present invention.

It is noted that the compounds of the present invention may also possess inhibitory activity for other aspartyl proteases (e.g., pepsin, gastricsin, napsin, BACE 1 & 2 and cathepsin D and E) and thus may be used to address disease states associated with these other family members. In addition, the compounds of the present invention may be useful as inhibitors of plasmepsins to treat malaria and as inhibitors of Candida albicans secreted aspartyl proteases to treat fungal infections.

In one embodiment, Renin inhibitors of the present invention comprise:

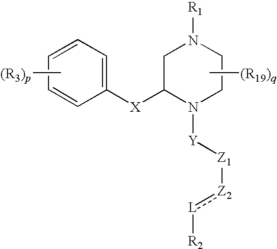

wherein:
p is selected from the group consisting of 0, 1, 2, 3, 4 and 5;
q is selected from the group consisting of 0, 1, 2 and 3;
L is absent or is a linker providing 1, 2 or 3 atom separation between Z₂ and R₂, wherein the atoms of the linker providing the separation are selected from the group consisting of carbon, oxygen, nitrogen, and sulfur;
X is —(CR₄R₅)ₙ—, where n is selected from the group consisting of 1 and 2;
Y is selected from the group consisting of —CO— and —SO₂—;
Z₁ is selected from the group consisting of CR₆R₇, NR₈, O and S;
Z₂ is selected from the group consisting of CR₉R₁₀, NR₁₁, O and S, or Z₂ and one or more of the atoms of L providing the separation are taken together to form a 3, 4, 5, 6 or 7 membered ring;
R₁ is selected from the group consisting of hydrogen and a substituent convertible in vivo to hydrogen;
R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;
R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀) alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted,
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R₆ and R₇ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, sulfonyl(C₁₋₃)alkyl, sulfinyl(C₁₋₃)alkyl, amino (C₁₋₁₀) alkyl and imino(C₁₋₃)alkyl, each substituted or unsubstituted, with the proviso that R₇ is absent when the atom to which it is bound forms part of a double bond, or R₆ and R₇ are taken together to form a ring;
R₈ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted;
R₉ and R₁₀ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together to form a ring, with the proviso that R₁₀ is absent when the atom to which it is bound forms part of a double bond;
R₁₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₁ is absent when the atom to which it is bound forms part of a double bond; and
R₁₉ is selected from the group consisting of hydrogen, methyl and fluoromethyl,
with the proviso that Z₁ is not —O— when Y is —CO—, R₁ is hydrogen, X is —CH₂—, R₂ is phenyl, and Z₂ is —CH₂—.

In one variation, Renin inhibitors of the present invention comprise:

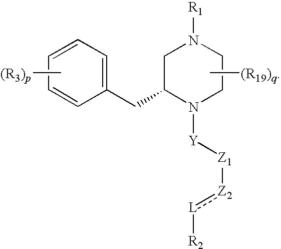

In another embodiment, Renin inhibitors of the present invention comprise:

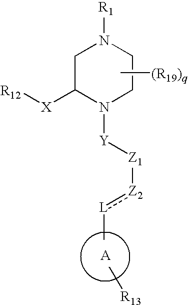

wherein:
q is selected from the group consisting of 0, 1, 2 and 3;
L is absent or is a linker providing 1, 2 or 3 atom separation between Z₂ and R₂, wherein the atoms of the linker providing the separation are selected from the group consisting of carbon, oxygen, nitrogen, and sulfur;
X is —(CR₄R₅)ₙ—, where n is selected from the group consisting of 1 and 2;
Y is selected from the group consisting of —CO— and —SO₂—;
Z₁ is selected from the group consisting of CR₆R₇, NR₈, O and S;
Z₂ is selected from the group consisting of CR₉R₁₀, NR₁₁, O and S, or Z₂ and one or more of the atoms of L providing the separation are taken together to form a 3, 4, 5, 6 or 7 membered ring;
R₁ is selected from the group consisting of hydrogen and a substituent convertible in vivo to hydrogen;
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R₆ and R₇ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, sulfonyl(C₁₋₃)alkyl, sulfinyl(C₁₋₃)alkyl, amino (C₁₋₁₀) alkyl and imino(C₁₋₃)alkyl, each substituted or unsubstituted, with the proviso that R₇ is absent when the atom to which it is bound forms part of a double bond, or R₆ and R₇ are taken together to form a ring;
R₈ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted;
R₉ and R₁₀ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together to form a ring, with the proviso that R₁₀ is absent when the atom to which it is bound forms part of a double bond;
R₁₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₁₀)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₁ is absent when the atom to which it is bound forms part of a double bond;
R₁₂ is a substituted or unsubstituted (C₄₋₇)aryl;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R₁₉ is selected from the group consisting of hydrogen, methyl and fluoromethyl,
with the proviso that Z₁ is not —O— when Y is —CO—, R₁ is hydrogen, X is —CH₂—, R₁₂ is phenyl, Z₂ is —CH₂—, ring A is phenyl and R₁₃ is hydrogen.

In one variation, Renin inhibitors of the present invention comprise:

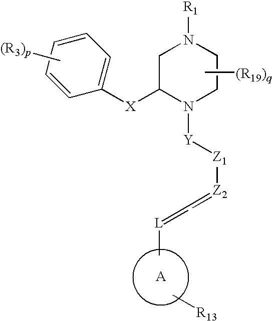

wherein:
p is selected from the group consisting of 0, 1, 2, 3, 4, and 5; and
R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted.

In another variation, Renin inhibitors of the present invention comprise:

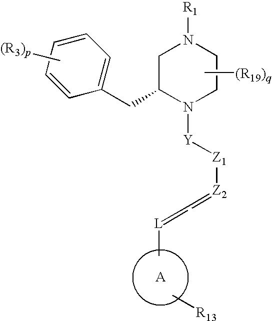

In still another variation, Renin inhibitors of the present invention comprise:

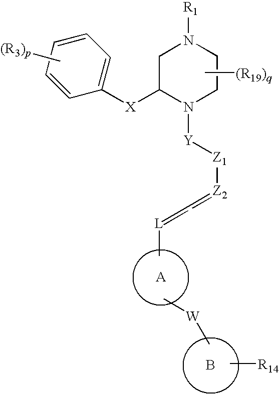

wherein:
p is selected from the group consisting of 0, 1, 2, 3, 4 and 5;
W is absent or selected from the group consisting of CR₁₅R₁₆, NR₁₇, O and S;
R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₁₀)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, with the proviso that R₁₆ is absent when the atom to which it is bound forms part of a double bond; and
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₇ is absent when the atom to which it is bound forms part of a double bond.

In yet another variation, Renin inhibitors of the present invention comprise:

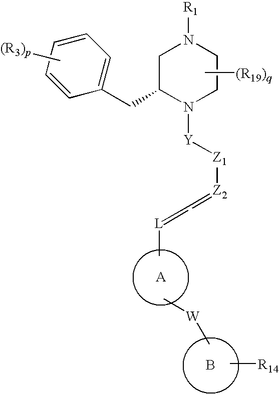

In yet another embodiment, Renin inhibitors of the present invention comprise:

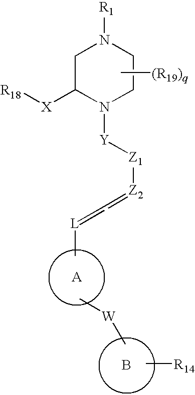

wherein:
q is selected from the group consisting of 0, 1, 2 and 3;
L is absent or is a linker providing 1, 2 or 3 atom separation between Z₂ and ring A, wherein the atoms of the linker providing the separation are selected from the group consisting of carbon, oxygen, nitrogen, and sulfur;
W is absent or selected from the group consisting of CR₁₅R₁₆, NR₁₇, O and S;
X is —(CR₄R₅)ₙ—, where n is selected from the group consisting of 1 and 2;
Y is selected from the group consisting of —CO— and —SO₂—;
Z₁ is selected from the group consisting of CR₆R₇ and NR₈, O and S;
Z₂ is selected from the group consisting of CR₉R₁₀ and NR₁₁, O and S, or Z₂ and one or more of the atoms of L providing the separation are taken together to form a 3, 4, 5, 6 or 7 membered ring;
R₁ is selected from the group consisting of hydrogen and a substituent convertible in vivo to hydrogen;
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R₆ and R₇ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, sulfonyl(C₁₋₃)alkyl, sulfinyl(C₁₋₃)alkyl, amino (C₁₋₁₀) alkyl and imino(C₁₋₃)alkyl, each substituted or unsubstituted, with the proviso that R₇ is absent when the atom to which it is bound forms part of a double bond, or R₆ and R₇ are taken together to form a ring;
R₈ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₁₀)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted;
R₉ and R₁₀ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together to form a ring, with the proviso that R₁₀ is absent when the atom to which it is bound forms part of a double bond;
R₁₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₁₀)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₁ is absent when the atom to which it is bound forms part of a double bond;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, with the proviso that R₁₆ is absent when the atom to which it is bound forms part of a double bond;
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₁₀)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₇ is absent when the atom to which it is bound forms part of a double bond;
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R₁₉ is selected from the group consisting of hydrogen, methyl and fluoromethyl.

In one variation of each of the above embodiments and variations, X is selected from the group consisting of —CH₂— and —CH₂CH₂—.

In another variation of each of the above embodiments and variations, R₄ is selected from the group consisting of hydrogen and halo.

In still another variation of each of the above embodiments and variations, R₅ is selected from the group consisting of hydrogen and halo.

In yet another variation of each of the above embodiments and variations, Z₁ is CR₆R₇.

In a further variation of each of the above embodiments and variations, Z₂ is CR₉R₁₀.

In still a further variation of each of the above embodiments and variations, R₆ is hydrogen.

In yet a further variation of each of the above embodiments and variations, R₇ is hydrogen.

In another variation of each of the above embodiments and variations, R₈ is selected from the group consisting of hydrogen and substituted or unsubstituted (C₁₋₃)alkyl.

In still another variation of each of the above embodiments and variations, R₉ is selected from the group consisting of hydrogen, hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted.

In yet another variation of each of the above embodiments and variations, R₁₀ is hydrogen.

In a further variation of each of the above embodiments and variations, R₁₁ is selected from the group consisting of hydrogen and substituted or unsubstituted (C₁₋₃)alkyl.

In still a further variation of each of the above embodiments and variations, R₁ is hydrogen.

In yet a further variation of each of the above embodiments and variations, R₁₂ is a substituted or unsubstituted phenyl.

In another variation of each of the above embodiments and variations, R₃ is selected from the group consisting of

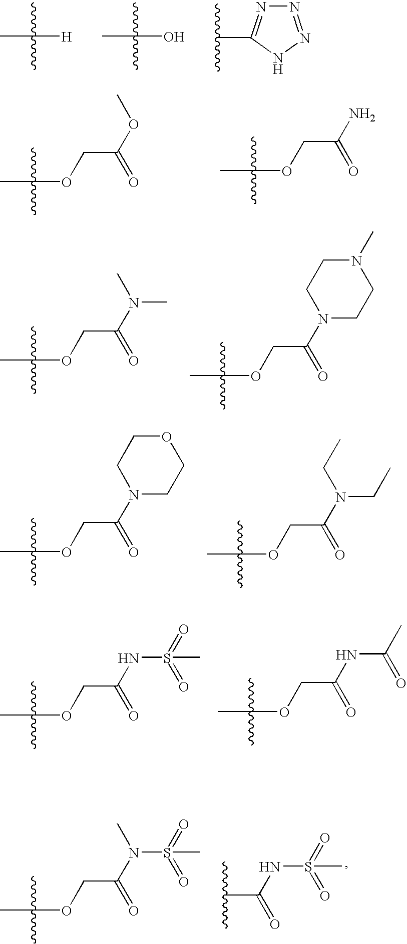

each substituted or unsubstituted.

In still another variation of each of the above embodiments and variations, R₂ is selected from the group consisting of phenyl and hetero(C₁₋₅)aryl, each substituted or unsubstituted. In yet another variation, R₂ is substituted with a substituent selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted. In a further variation, R₂ is substituted with a substituent selected from the group consisting of

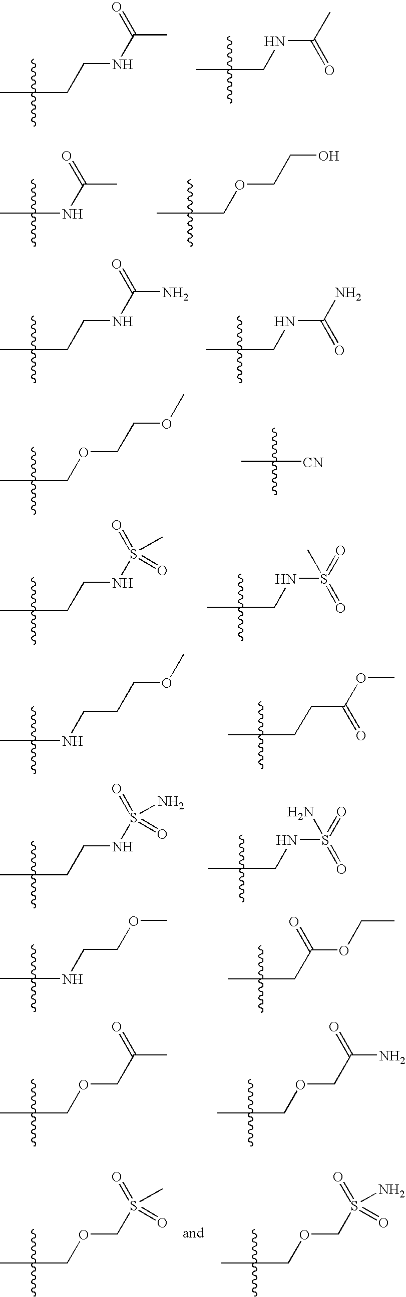

each substituted or unsubstituted.

In still a further variation of each of the above embodiments and variations, ring A is selected from the group consisting of phenyl and hetero(C₁₋₅)aryl, each substituted or unsubstituted.

In yet a further variation of each of the above embodiments and variations, R₁₃ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted. In another variation, R₁₃ is selected from the group consisting of

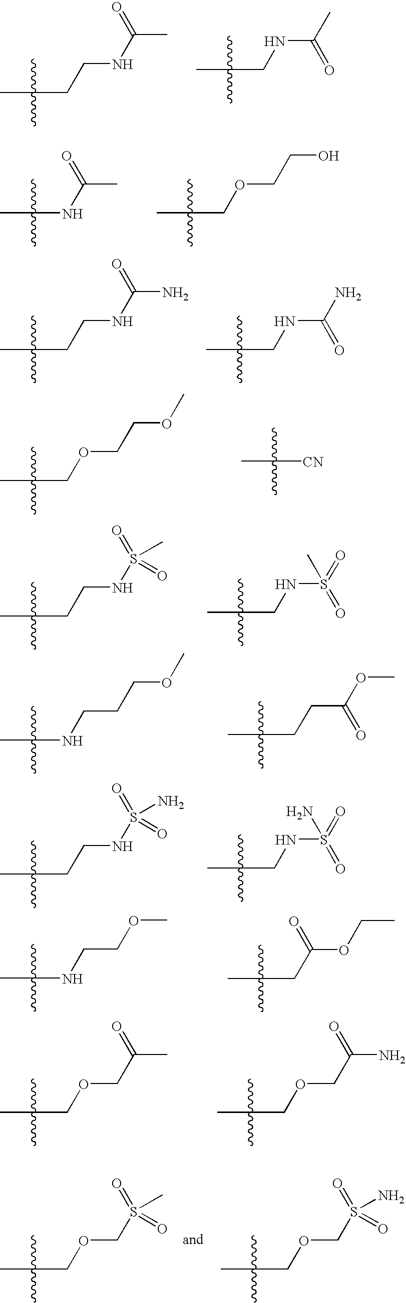

each substituted or unsubstituted.

In yet another variation of each of the above embodiments and variations, W is selected from the group consisting of CR₁₅R₁₆ where R₁₅ and R₁₆ are each independently selected from the group consisting of hydrogen, halo, hydroxyl and substituted or unsubstituted (C₁₋₃)alkyl; NH; and O.

In a further variation of each of the above embodiments and variations, R₁₅ is selected from the group consisting of hydrogen, hydroxyl, halo and substituted or unsubstituted (C₁₋₃)alkyl.

In still a further variation of each of the above embodiments and variations, R₁₆ is selected from the group consisting of hydrogen, hydroxyl, halo and substituted or unsubstituted (C₁₋₃)alkyl.

In yet a further variation of each of the above embodiments and variations, R₁₇ is selected from the group consisting of

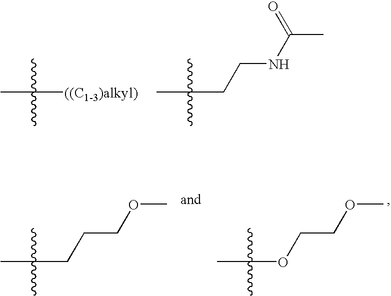

each substituted or unsubstituted.

In another variation of each of the above embodiments and variations, R₁₈ is selected from the group consisting of (C₁₋₆)alkyl, (C₃₋₇)cycloalkyl, (C₄₋₇)aryl and hetero(C₁₋₅)aryl, each substituted or unsubstituted.

In still another variation of each of the above embodiments and variations, ring B is selected from the group consisting of phenyl and hetero(C₁₋₁₀)aryl, each substituted or unsubstituted.

In yet another variation of each of the above embodiments and variations, R₁₄ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, amido(C₁₋₁₀)alkyl, alkoxyalkoxy(C₁₋₁₀)alkyl, alkoxy(C₁₋₁₀)alkyl and alkoxyalkoxy, each substituted or unsubstituted. In a further variation, R₁₄ is selected from the group consisting of

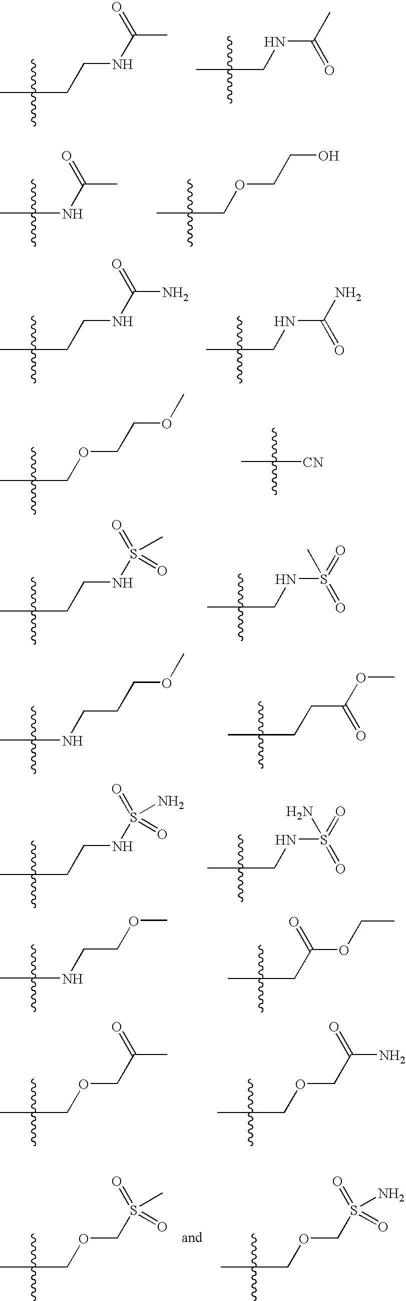

each substituted or unsubstituted.

In still a further variation of each of the above embodiments and variations, L is absent.

In yet a further variation of each of the above embodiments and variations, Y is —CO—.

In another variation of the above embodiments and variations, p is 0. In still another variation of the above embodiments and variations, p is 1. In yet another variation of the above embodiments and variations, q is 0. In a further variation of the above embodiments and variations, q is 1. In one particular variation, the piperazine ring is substituted with at least one R₁₉ at the 2-position of the ring. In still a further variation of the above embodiments and variations, at least one R₁₉ is methyl.

Particular examples of compounds according to the present invention include, but are not limited to:
(R)-1-(2-benzylpiperazin-1-yl)-3,3-diphenylpropan-1-one;
(R)-1-(2-benzylpiperazin-1-yl)-2-(4′-fluorobiphenyl-2-yl)ethanone;
(R)-1-(2-benzylpiperazin-1-yl)-2-(2-bromophenoxy)ethanone;
(R)-1-(2-benzylpiperazin-1-yl)-4-phenylbutane-1,4-dione;
(R)-1-(2-benzylpiperazin-1-yl)-3-phenylpropan-1-one;
1-((R)-2-benzylpiperazin-1-yl)-2,3-diphenylpropan-1-one;
(R)-1-(2-benzylpiperazin-1-yl)-3-(2-phenoxyphenyl)propan-1-one;
(R)-1-(2-benzylpiperazin-1-yl)-2-(2-phenoxyphenyl)ethanone;
1-((R)-2-benzylpiperazin-1-yl)-2-(2′-methoxybiphenyl-2-yl)ethanone;
(R)—N-benzhydryl-2-benzylpiperazine-1-carboxamide;
1-((R)-2-benzylpiperazin-1-yl)-2-(2′-chlorobiphenyl-2-yl)ethanone;
(R)-2-benzyl-1-(phenethylsulfonyl)piperazine;
2-(3-((R)-2-benzylpiperazin-1-yl)-3-oxo-1-p-tolylpropyl)isoindolin-1-one;
(R)-1-(2-benzylpiperazin-1-yl)-2-(naphthalen-1-yloxy)ethanone;
1-((R)-2-benzylpiperazin-1-yl)-3-(furan-2-yl)-4-phenylbutan-1-one;
(R)-2-benzyl-1-(2,2-diphenylethylsulfonyl)piperazine;
(R)-1-((R)-2-benzylpiperazin-1-yl)-3-phenylbutan-1-one;
(S)-1-((R)-2-benzylpiperazin-1-yl)-3-phenylbutan-1-one;
(R)-1-(2-benzylpiperazin-1-yl)-4-phenylbutan-1-one;
(R)-1-(2-benzylpiperazin-1-yl)-3-(2-(4-chlorophenoxy)phenyl)propan-1-one;
1-((R)-2-benzylpiperazin-1-yl)-2-(2,2-dimethyl-4-phenyltetrahydro-2H-pyran-4-yl)ethanone;
(R)-2-(3-(2-benzylpiperazin-1-yl)-3-oxopropyl)phenyl benzoate;
(R)-2-(2-(3-(2-benzylpiperazin-1-yl)-3-oxopropyl)phenoxy)benzonitrile;
(R)-1-(2-benzylpiperazin-1-yl)-3-(2-(m-tolyloxy)phenyl)propan-1-one;
(R)—N-(2-(2-(3-(2-benzylpiperazin-1-yl)-3-oxopropyl)phenoxy)phenethyl)acetamide;
(R)—N-(3-(2-(3-(2-benzylpiperazin-1-yl)-3-oxopropyl)phenoxy)phenyl)acetamide;
(R)—N-(2-(2-(3-(2-benzylpiperazin-1-yl)-3-oxopropyl)phenoxy)benzyl)acetamide;
(R)-1-(2-benzylpiperazin-1-yl)-3-(2-(3-(dimethylamino)phenoxy)phenyl)propan-1-one;
(R)-1-(2-benzylpiperazin-1-yl)-3-(2-(3-ethoxyphenoxy)phenyl)propan-1-one;
(R)-3-(2-(3-(2-benzylpiperazin-1-yl)-3-oxopropyl)phenoxy)-N-ethylbenzamide;
(R)—N-((2-(2-(3-(2-benzylpiperazin-1-yl)-3-oxopropyl)phenoxy)pyridin-3-yl)methyl)acetamide;
(R)—N-((2-(2-(3-(2-benzylpiperazin-1-yl)-3-oxopropyl)phenoxy)-4,6-dimethylpyridin-3-yl)methyl)acetamide;
(R)—N-(2-(2-(3-(2-benzylpiperazin-1-yl)-3-oxopropyl)phenoxy)-5-methylbenzyl)acetamide;
(R)—N-(2-(2-(3-(2-benzylpiperazin-1-yl)-3-oxopropyl)phenoxy)-5-methoxybenzyl)acetamide;
(R)-1-(2-benzylpiperazin-1-yl)-3-(2-(p-tolyloxy)phenyl)propan-1-one;
(R)-1-(2-benzylpiperazin-1-yl)-3-(2-(4-methoxyphenoxy)phenyl)propan-1-one;
(R)—N-((2-(2-(3-(2-benzylpiperazin-1-yl)-3-oxopropyl)-4-methylphenoxy)-6-methylpyridin-3-yl)methyl)acetamide;
(R)—N-(2-(2-(3-(2-benzylpiperazin-1-yl)-3-oxopropyl)-4-methylphenoxy)-5-methylbenzyl)acetamide;
(R)—N-((2-(2-(3-(2-benzylpiperazin-1-yl)-3-oxopropyl)-5-methylphenoxy)-6-methylpyridin-3-yl)methyl)acetamide;
(R)—N-(2-(2-(3-(2-benzylpiperazin-1-yl)-3-oxopropyl)-5-methylphenoxy)-5-(trifluoromethyl)benzyl)acetamide;
(R)—N-(2-(2-(3-(2-benzylpiperazin-1-yl)-3-oxopropyl)-5-methylphenoxy)-5-methylbenzyl)acetamide;
(R)—N-(2-(2-(3-(2-benzylpiperazin-1-yl)-3-oxopropyl)-4-methylphenoxy)-1-methylphenethyl)acetamide;
(R)—N-(2-(2-(3-(2-benzylpiperazin-1-yl)-3-oxopropyl)-4-methylphenoxy)phenethyl)acetamide;
(R)—N-(2-(2-(3-(2-benzylpiperazin-1-yl)-3-oxopropyl)-5-methylphenoxy)-1-methylphenethyl)acetamide;
(R)—N-(2-(2-(3-(2-benzylpiperazin-1-yl)-3-oxopropyl)-5-methylphenoxy)phenethyl)acetamide;
(R)—N-(2-(2-(3-(2-benzylpiperazin-1-yl)-3-oxopropyl)-4-methylphenoxy)-5-(trifluoromethyl)benzyl)acetamide;
(R)—N-(3-(2-(2-(3-(2-benzylpiperazin-1-yl)-3-oxopropyl)phenoxy)phenyl)propyl)acetamide;
(R)—N-(2-(2-(3-(2-benzylpiperazin-1-yl)-3-oxopropyl)phenoxy)-5-methylphenethyl)acetamide;
2-(3-((R)-2-benzylpiperazin-1-yl)-3-oxopropyl)-2-phenylcyclohexanone;
(R)-3-(2-benzylphenyl)-1-(2-benzylpiperazin-1-yl)propan-1-one;
(R)-1-(2-benzylpiperazin-1-yl)-3-(4-(4-chlorophenyl)-2-methyloxazol-5-yl)propan-1-one;
(R)-3-(3-(2-benzylpiperazin-1-yl)-3-oxopropyl)-2H-benzo[b][1,4]oxazin-2-one;
(R)-1-(2-benzylpiperazin-1-yl)-3-(2-(4-chlorobenzyl)phenyl)propan-1-one;
(R)-1-(2-benzylpiperazin-1-yl)-2-(2-methoxyphenoxy)ethanone;
(R)-1-(2-benzylpiperazin-1-yl)-3-(diphenylamino)propan-1-one;
(R)-4-(3-(2-benzylpiperazin-1-yl)-3-oxopropyl)-2-methoxyphenyl acetate;
(R)-4-(2-benzylpiperazin-1-yl)-4-oxo-N,N-diphenylbutanamide;
1-((R)-2-benzylpiperazin-1-yl)-3-(2,2-dimethyltetrahydro-2H-pyran-4-yl)-3-phenylpropan-1-one;
(R)-2-(3-benzylphenoxy)-1-(2-benzylpiperazin-1-yl)ethanone;
(R)-1-(2-benzylpiperazin-1-yl)-3-(4,5-diphenyloxazol-2-yl)propan-1-one;
(R)—N-(1-(2-(2-benzylpiperazin-1-yl)-2-oxoethyl)cyclohexyl)benzamide;
(R)-2-(benzhydrylthio)-1-(2-benzylpiperazin-1-yl)ethanone;
(R)-3-(2-(2-(3-(2-benzylpiperazin-1-yl)-3-oxopropyl)phenoxy)phenyl)propanenitrile;
(R)-methyl 3-(2-(2-(3-(2-benzylpiperazin-1-yl)-3-oxopropyl)phenoxy)phenyl)propanoate;
(R)-1-(2-(4-hydroxybenzyl)piperazin-1-yl)-3-(2-phenoxyphenyl)propan-1-one;
(R)-methyl 2-(4-((1-(3-(2-phenoxyphenyl)propanoyl)piperazin-2-yl)methyl)phenoxy)acetate;
(R)-2-(4-((1-(3-(2-phenoxyphenyl)propanoyl)piperazin-2-yl)methyl)phenoxy)acetamide;
(R)—N,N-dimethyl-2-(4-((1-(3-(2-phenoxyphenyl)propanoyl)piperazin-2-yl)methyl)phenoxy)acetamide;
(R)-4-((1-(3-(2-phenoxyphenyl)propanoyl)piperazin-2-yl)methyl)phenyl 4-methylpiperazine-1-carboxylate;
(R)-4-((1-(3-(2-phenoxyphenyl)propanoyl)piperazin-2-yl)methyl)phenyl morpholine-4-carboxylate;
(R)-4-((1-(3-(2-phenoxyphenyl)propanoyl)piperazin-2-yl)methyl)phenyl diethylcarbamate;
(R)-1-(2-benzylpiperazin-1-yl)-3-(2-(3-hydroxypropoxy)phenyl)propan-1-one;
(R)-1-(2-benzylpiperazin-1-yl)-3-(2-(3-methoxypropoxy)phenyl)propan-1-one;
(R)-1-(2-benzylpiperazin-1-yl)-3-(2-(3-(2-methoxyethoxy)propoxy)phenyl)propan-1-one;
(R)-1-(2-benzylpiperazin-1-yl)-3-(2-(3-(hydroxymethyl)phenoxy)phenyl)propan-1-one;
(R)-3-(2-(3-acetylphenoxy)phenyl)-1-(2-benzylpiperazin-1-yl)propan-1-one;
(R)-3-(2-(3-(2-benzylpiperazin-1-yl)-3-oxopropyl)phenoxy)benzamide;
(R)—N-(2-(2-(3-(2-benzylpiperazin-1-yl)-3-oxopropyl)phenoxy)phenethyl)propionamide;
(R)—N-(2-(2-(3-(2-(4-hydroxybenzyl)piperazin-1-yl)-3-oxopropyl)phenoxy)phenethyl)acetamide;
(R)-4-((1-(3-(2-(2-(2-acetamidoethyl)phenoxy)phenyl)propanoyl)piperazin-2-yl)methyl)phenyl morpholine-4-carboxylate;
(R)-2-(4-((1-(3-(2-(2-(2-acetamidoethyl)phenoxy)phenyl)propanoyl)piperazin-2-yl)methyl)phenoxy)acetamide;
(R)-3-(2-(2-(3-(2-benzylpiperazin-1-yl)-3-oxopropyl)phenoxy)phenyl)propanoic acid;
(R)—N-(2-(2-(3-(2-benzylpiperazin-1-yl)-3-oxopropyl)benzyl)phenethyl)acetamide;
(R)-3-(2-(2-(3-(2-benzylpiperazin-1-yl)-3-oxopropyl)phenoxy)phenyl)propanamide;
(R)-1-(2-(4-((1H-tetrazol-5-yl)methoxy)benzyl)piperazin-1-yl)-3-(2-phenoxyphenyl)propan-1-one;
(R)-2-(4-((1-(3-(2-(2-(2-acetamidoethyl)benzyl)phenyl)propanoyl)piperazin-2-yl)methyl)phenoxy)acetamide;
(R)-2-(4-((1-(3-(2-(2-(2-acetamidoethyl)benzyl)phenyl)propanoyl)piperazin-2-yl)methyl)phenoxy)acetic acid;
(R)-1-(2-benzylpiperazin-1-yl)-3-(2-(2-(hydroxymethyl)phenoxy)phenyl)propan-1-one;
(R)—N-(2-(2-(3-(2-(4-hydroxybenzyl)piperazin-1-yl)-3-oxopropyl)benzyl)phenethyl)acetamide; and
(R)-2-(4-((1-(3-(2-phenoxyphenyl)propanoyl)piperazin-2-yl)methyl)phenoxy)acetic acid;
(R)-2-(4-((1-(3-(2-(2-(2-acetamidoethyl)benzyl)phenyl) propanoyl)piperazin-2-yl)methyl)phenoxy)-N-(methylsulfonyl)acetamide
(R)-Methyl 2-(4-((1-(3-(2-(2-(2-acetamidoethyl)benzyl)phenyl) propanoyl)piperazin-2-yl)methyl)phenoxy)acetate
(R)-4-((1-(3-(2-(2-(2-acetamidoethyl)benzyl)phenyl)propanoyl)piperazin-2-yl)methyl)-N-(methylsulfonyl)benzamide
(R)—N-(2-(2-(3-(2-(4-(1H-tetrazol-5-yl)benzyl)piperazin-1-yl)-3-oxopropyl)benzyl)phenethyl)acetamide

It is noted that the compounds of the present invention may be in the form of a pharmaceutically acceptable salt, biohydrolyzable ester, biohydrolyzable amide, biohydrolyzable carbamate, solvate, hydrate or prodrug thereof. For example, the compound optionally comprises a substituent that is convertible in vivo to a different substituent such as a hydrogen.

It is further noted that the compound may be present in a mixture of stereoisomers, or the compound may comprise a single stereoisomer.

The present invention also provides a pharmaceutical composition comprising as an active ingredient a compound according to any one of the above embodiments and variations. In one particular variation, the composition is a solid formulation adapted for oral administration. In another particular variation, the composition is a liquid formulation adapted for oral administration. In yet another particular variation, the composition is a tablet. In still another particular variation, the composition is a liquid formulation adapted for parenteral administration.

In another of its aspects, there is provided a pharmaceutical composition comprising a compound according to any one of the above embodiments and variations, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In yet another of its aspects, there is provided a kit comprising a compound of any one of the above embodiments and variations; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound of any one of the above embodiments and variations; and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound of any one of the above embodiments and variations to a subject.

In another of its aspects, there is provided a method of inhibiting Renin comprising contacting Renin with a compound of any one of the above embodiments and variations.

In yet another of its aspects, there is provided a method of inhibiting Renin comprising causing a compound of any one of the above embodiments and variations to be present in a subject in order to inhibit Renin in vivo.

In a further of its aspects, there is provided a method of inhibiting Renin comprising administering a first compound to a subject that is converted in vivo to a second compound wherein the second compound inhibits Renin in vivo, the second compound being a compound according to any one of the above embodiments and variations.

In another of its aspects, there is provided a method of treating a disease state for which Renin possesses activity that contributes to the pathology and/or symptomology of the disease state, the method comprising causing a compound of any one of the above embodiments and variations to be present in a subject in a therapeutically effective amount for the disease state.

In yet another of its aspects, there is provided a method of treating a disease state for which Renin possesses activity that contributes to the pathology and/or symptomology of the disease state, the method comprising administering a compound of any one of the above embodiments and variations to a subject, wherein the compound is present in the subject in a therapeutically effective amount for the disease state.

In a further of its aspects, there is provided a method of treating a disease state for which Renin possesses activity that contributes to the pathology and/or symptomology of the disease state, the method comprising administering a first compound to a subject that is converted in vivo to a second compound wherein the second compound inhibits Renin in vivo, the second compound being a compound according to any one of the above embodiments and variations.

In one variation of each of the above embodiments and variations, the disease state is selected from the group consisting of cardiovascular disease, hypertension, congestive heart failure, myocardial infarction, renal protection, inflammation, neurological disease and cancer.

Another aspect of the invention relates to methods for preparing Renin inhibitors of the invention. In one embodiment, the method comprising

coupling a compound having the formula

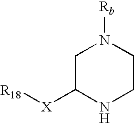

to a compound of the formula R_{d}—Y-J,

under conditions that form a reaction product of the formula

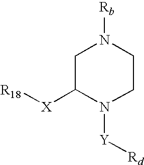

wherein
J is a leaving group;
X is —(CR₄R₅)ₙ—, where n is selected from the group consisting of 1 and 2;
Y is selected from the group consisting of —C(O)— and —S(O)₂—;
R_{b} is selected from the group consisting of R₁, benzyl and Boc;
R_{d} is substituted alkyl or substituted heteroalkyl;
R₁ is selected from the group consisting of hydrogen and a substituent convertible in vivo to hydrogen;
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In some variations of the preceding embodiment, Y is —C(O)—.

In other variations of the preceding embodiment, Y is —S(O)₂—.

In some variations of the preceding embodiment and variations, R_{d} is selected from the group consisting of

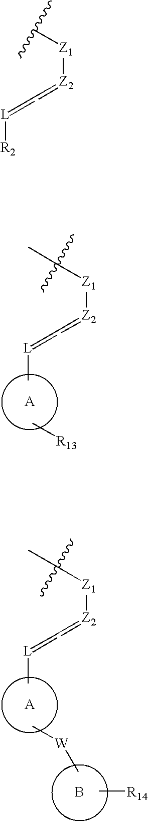

wherein
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
L is absent or is a linker providing 1, 2 or 3 atom separation between Z₂ and R₂ or ring A, wherein the atoms of the linker providing the separation are selected from the group consisting of carbon, oxygen, nitrogen, and sulfur;
W is absent or selected from the group consisting of CR₁₅R₁₆, NR₁₇, O and S;
Z₂ is selected from the group consisting of CR₆R₇ and NR₁₁, O and S;
Z₂ is selected from the group consisting of CR₉R₁₀ and NR₁₁, O and S, or Z₂ and one or more of the atoms of L providing the separation are taken together to form a 3, 4, 5, 6 or 7 membered ring;
R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;
R₆ and R₇ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, sulfonyl(C₁₋₃)alkyl, sulfinyl(C₁₋₃)alkyl, amino (C₁₋₁₀) alkyl and imino(C₁₋₃)alkyl, each substituted or unsubstituted, with the proviso that R₇ is absent when the atom to which it is bound forms part of a double bond, or R₆ and R₇ are taken together to form a ring;
R₈ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₁₀)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted;
R₉ and R₁₀ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together to form a ring, with the proviso that R₁₀ is absent when the atom to which it is bound forms part of a double bond;
R₁₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₁₀)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₁₀)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₁ is absent when the atom to which it is bound forms part of a double bond;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₁₀)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, with the proviso that R₁₆ is absent when the atom to which it is bound forms part of a double bond; and
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₇ is absent when the atom to which it is bound forms part of a double bond.

In some variations of the preceding embodiment and variations, L is absent.

In some variations of the preceding embodiment and variations, W is absent, or is selected from the group consisting of —CR₁₅R₁₆—, and —O—, wherein R₁₅ and R₁₆ are each independently selected from the group consisting of hydrogen, halo, hydroxyl and substituted and unsubstituted (C₁₋₃)alkyl, NH, and O. In some other variations, W is CH₂ or O.

In some variations of the preceding embodiment and variations, Z₁ is CR₆R₇. In some variations, R₆ and R₇ are both hydrogen.

In some variations of the preceding embodiment and variations, Z₂ is CR₉R₁₀, or O.

In some variations of the preceding embodiment and variations, R₉ is selected from the group consisting of hydrogen, hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted.

In some variations of the preceding embodiment and variations, R₁₀ is hydrogen.

In some other variations of the preceding embodiment and variations, Z₂ is CH₂.

In some variations of the preceding embodiment and variations, R₂ is selected from the group consisting of phenyl and (C₁₋₅)heteroaryl, each substituted or unsubstituted.

In some variations, R₂ is substituted with a substituent selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, and cycloalkoxy(C₃₋₆)carbonylalkyl(C₁₋₁₀)alkyl, each substituted or unsubstituted.

In other variations, R₂ is substituted with a substituent selected from the group consisting of

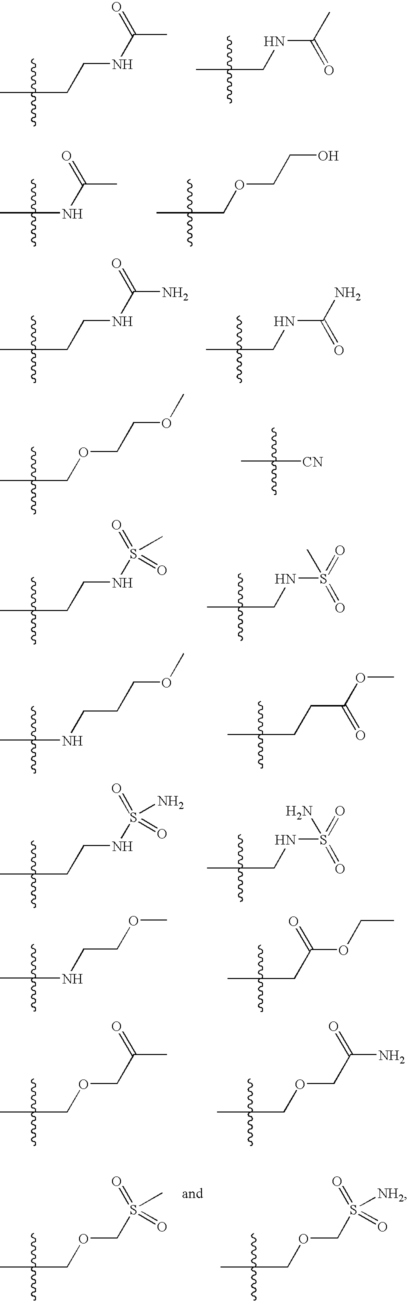

each substituted or unsubstituted.

In another embodiment of the method for the preparation of the Renin inhibitors of the invention, the method comprises

coupling a compound having the formula

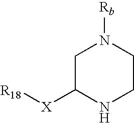

to a compound of the formula

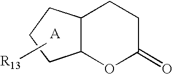

under conditions that form a compound of the formula

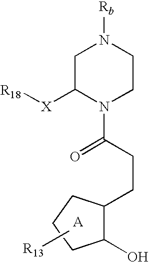

wherein
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
X is —(CR₄R₅)ₙ—, where n is selected from the group consisting of 1 and 2;
R_{b} is selected from the group consisting of R₁, benzyl and Boc;
R₁ is selected from the group consisting of hydrogen and a substituent convertible in vivo to hydrogen;
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

In one variation of the preceding embodiment, the method further comprises:

reacting the compound having the formula

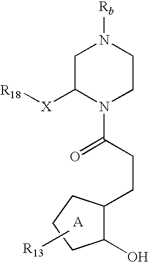

with a dihydrocoumarine derivative having the formula

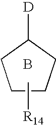

under conditions that form a compound having the formula

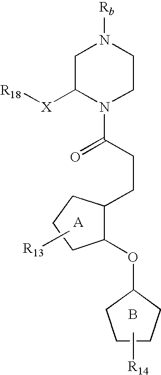

wherein
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.
D is selected from the group consisting of —F, —Cl and —Br; and
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted.

In some variations of the preceding two embodiments, the method further comprises:

coupling a compound of the formula

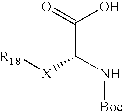

to another compound of the formula

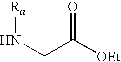

under conditions that form a compound having the formula

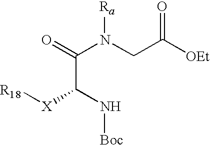

cyclizing the compound immediately above to form a compound having the formula

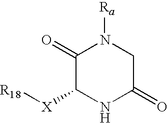

reducing the compound immediately above to a product having the formula

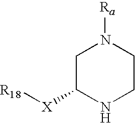

wherein
Rₐ is R₁ or benzyl.

In some variations of the preceding two embodiments and variations, R₅ is selected from the group consisting of (C₁₋₆)alkyl, (C₃₋₇)cycloalkyl, (C₄₋₇)aryl and hetero(C₁₋₅)aryl, each substituted or unsubstituted.

In other variations, R₁₈ is a substituted phenyl and the substituent is selected from the group consisting of

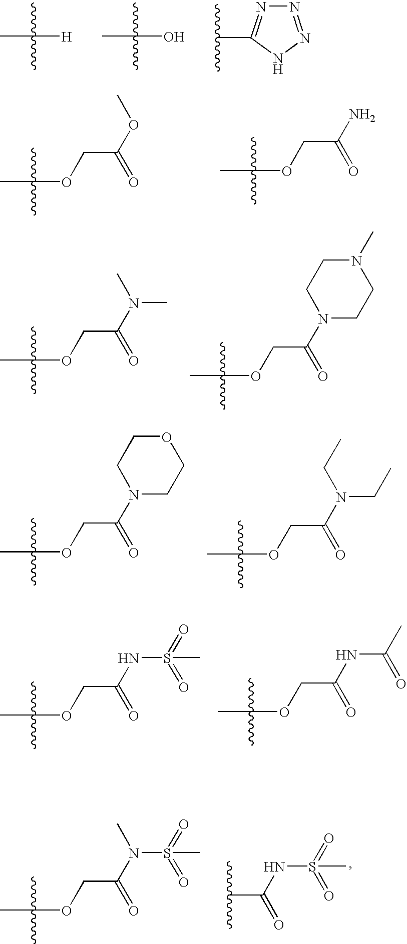

each substituted or unsubstituted.

In some variations of the preceding two embodiments and variations, R₄ and R₅ are each independently selected from the group consisting of hydrogen and halo.

In some other variations of the preceding two embodiments and variations, X is selected from the group consisting of —CH₂— and —CH₂CH₂—.

In still other variations of the preceding two embodiments and variations, ring A is selected from the group consisting of phenyl and hetero(C₁₋₅)aryl, each substituted or unsubstituted.

In still other variations of the preceding two embodiments and variations, R₁₃ is selected from the group consisting of

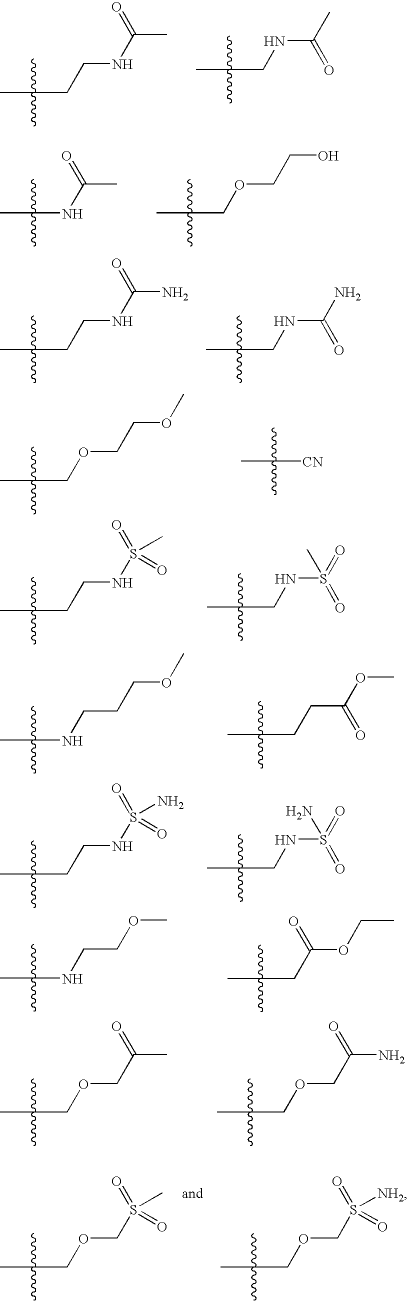

each substituted or unsubstituted.

In still other variations of the preceding two embodiments and variations, ring B is selected from the group consisting of phenyl and hetero(C₁₋₁₀)aryl, each substituted or unsubstituted.

In still other variations of the preceding two embodiments and variations, R₁₄ is selected from the group consisting of

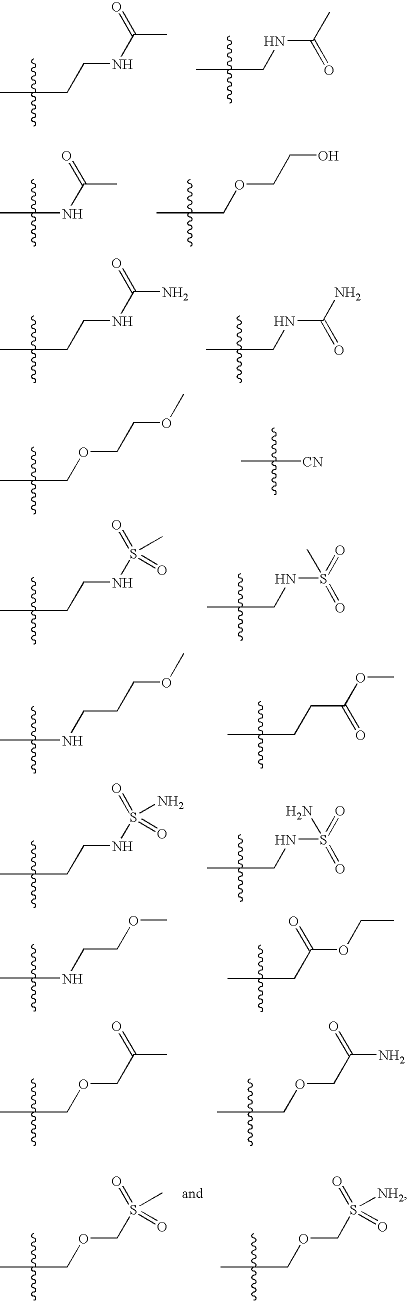

each substituted or unsubstituted.

Yet another aspect of the present invention relates to intermediates used for the preparation of the Renin inhibitors of the invention. In one embodiment, the intermediate is a compound of the formula

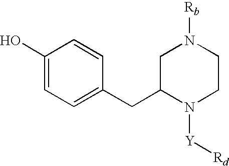

wherein
Y is selected from the group consisting of —C(O)— and —S(O)₂—;
R_{b} is selected from the group consisting of R₁, benzyl and Boc;
R_{d} is substituted alkyl or substituted heteroalkyl; and
R₁ is selected from the group consisting of hydrogen and a substituent convertible in vivo to hydrogen.

A compound of the formula

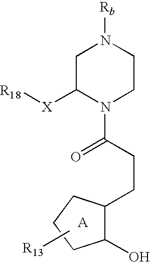

wherein
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
X is —(CR₄R₅)ₙ— where n is selected from the group consisting of 1 and 2;
R_{b} is selected from the group consisting of R₁, benzyl and a Boc group;
R₁ is selected from the group consisting of hydrogen and a substituent convertible in vivo to hydrogen;
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In another embodiment, the intermediate is a compound of the formula

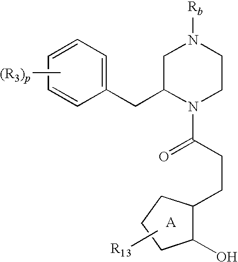

wherein
p is selected from the group consisting of 0, 1, 2, 3, 4 and 5;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R_{b} is selected from the group consisting of R₁, benzyl and Boc;
R₁ is selected from the group consisting of hydrogen and a substituent convertible in vivo to hydrogen;
R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀) alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted, and
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀) alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₁₀)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In still another embodiment of the intermediate is a compound of the formula

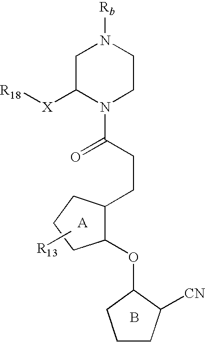

wherein
X is —(CR₄R₅)ₙ—, where n is selected from the group consisting of 1 and 2;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R_{b} is selected from the group consisting of R₁, benzyl and Boc;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In some variations of the preceding three embodiments, R₁₈ is a substituted phenyl.

In other variations, R₃ is selected from the group consisting of

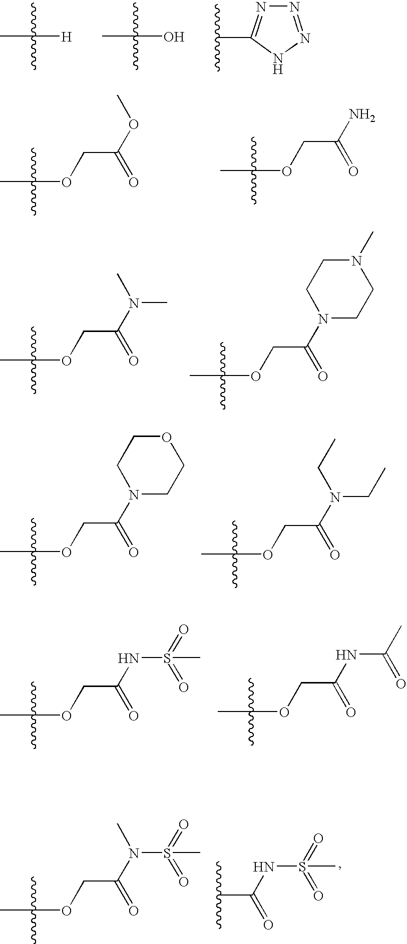

each substituted or unsubstituted.

In yet other variations, Y is —C(O)—.

The compound according to any one of claims 90-96, wherein R₁ is hydrogen, methyl or ethyl.

In yet other variations, ring A is selected from the group consisting of phenyl and hetero (C₁₋₅)aryl, each substituted or unsubstituted.

In yet other variations, R₁₃ is selected from the group consisting of

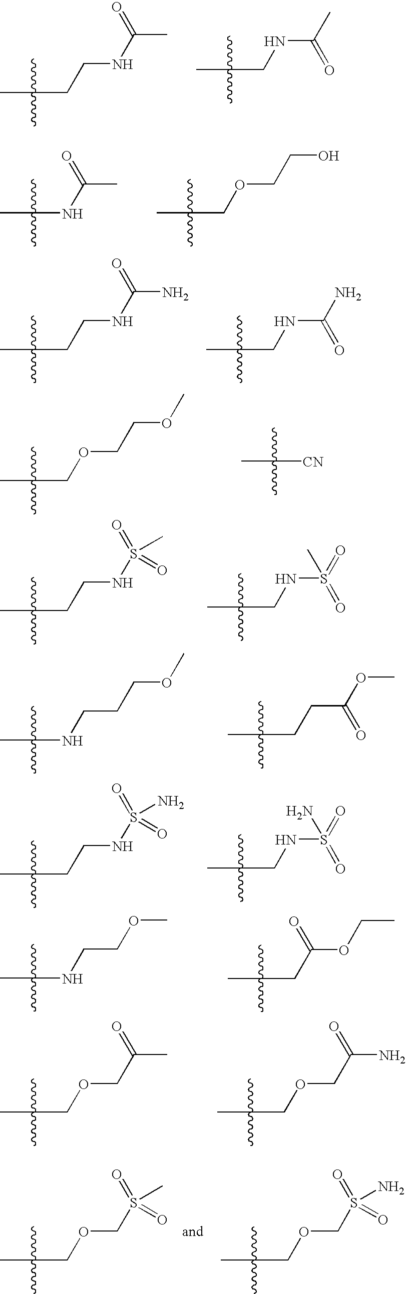

each substituted or unsubstituted.

In still other variations, ring B is selected from the group consisting of phenyl and hetero and (C₁₋₁₀)aryl, each substituted or unsubstituted.

### Salts, Hydrates, and Prodrugs of Renin Inhibitors

It should be recognized that the compounds of the present invention may be present and optionally administered in the form of salts, hydrates and prodrugs that are converted in vivo into the compounds of the present invention. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention that comprise basic nitrogen-containing groups may be quaternized with such agents as (C₁₋₄) alkyl halides, e.g., methyl, ethyl, iso-propyl and tert-butyl chlorides, bromides and iodides; di (C₁₋₄) alkyl sulfates, e.g., dimethyl, diethyl and diamyl sulfates; (C₁₋₁₈) alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water-soluble and oil-soluble compounds of the present invention.

N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

Compounds of the present invention may also be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

### Indications for Use of Renin Inhibitors

Renin inhibitors of the present invention may be used to treat and/or prevent high blood pressure, cardiovascular diseases, congestive heart failure, myocardial infarction, renal protection, inflammation, neurological disease and cancer.

Renin is a proteolytic enzyme synthesized and stored principally in the juxtaglomerular apparatus. When renin is released into the blood from the kidney, the renin-angiotensin-aldosterone system (“RAAS”) is activated. Renin acts on the alpha-2 globulin angiotensinogen (synthesized in the liver) to generate angiotensin I. This non-pressor decapeptide is converted to angiotensin II by angiotensin-converting enzyme (ACE). The major pharmacological effects of angiotensin II are vasoconstriction and stimulation of the adrenal cortex to release aldosterone, a hormone which causes sodium retention. Vasoconstriction and conservation of sodium both contribute to increased blood pressure. Angiotensin II also produces other physiological effects such as inhibiting renin secretion, increasing sympathetic nervous system activity, stimulating vasopressin secretion, causing a positive cardiac inotropic effect and modulating other hormonal systems. Thus, the renin-angiotensin system plays an important role in normal cardiovascular homeostasis and in some forms of elevated blood pressure (hypertension).

The reduction of the activity of renin in a subject through inhibition may therefore be used to therapeutically address the diseases and conditions caused by the overactivation of RAAS.

Thus, renin inhibiting compounds of the present invention may be used as agents for control of hypertension, may also be used to treat and/or prevent congestive heart failure and hyperaldosteronism, vascular diseases related to diabetes, and renal diseases such as acute or chronic renal failure. In addition, the renin inhibiting compounds may also be used as diagnostic agents for identification of cases of hypertension due to renin excess.

It is noted that the compounds of the present invention may also possess inhibitory activity for other aspartyl proteases (e.g., pepsin, gastricsin, napsin, BACE 1 & 2 and cathepsin D and E) and thus may be used to address disease states associated with these other family members.

In addition, the compounds of the present invention may be useful as inhibitors of plasmepsins to treat malaria and as inhibitors of Candida albicans secreted aspartyl proteases to treat fungal infections.

It is further noted that additional diseases beyond those disclosed herein may also be identified as the biological roles that renin and the RAAS system play in various pathways become more fully understood.

### Combination Therapy

A wide variety of therapeutic agents may have a therapeutic additive or synergistic effect when used in combination with renin inhibitors according to the present invention. Such therapeutic agents may additively or synergistically combine with the renin inhibitors to reduce or alleviate the effects and symptoms of cardiovascular disease.

The compounds according to the present invention may be used in combination with other therapeutic agents, wherein the cells are treated with a compound according to the present invention before, at the same time, and/or after the cells are treated with the one or more additional cardiovascular therapeutics; these treatments are referred to herein as combination therapy. It is noted that administration of one agent before another is referred to herein as sequential therapy, even if the agents are also administered together. It is noted that combination therapy is intended to cover methods where agents are administered before or after each other (sequential therapy) as well as when the agents are administered at the same time.

Representative classes of cardiovascular agents that may be used with the renin inhibitors of the present invention include, but are not limited to, diuretics, adrenergic blocking agents, vasodilators, calcium channel blockers, angiotensin converting enzyme (ACE) inhibitors, potassium channel activators, antiserotoninergic agents, thromboxane synthetase inhibitors, angiotensin II antagonists, angiotensin II receptor blockers and other agents useful for treating (in a human or other mammal) hypertension, congestive heart failure, or vascular diseases related to diabetes, or for treating renal diseases such as acute or chronic renal failure.

Representative diuretics include hydrochlorothiazide, polythiazide, piretanide, torasemide, bumetanide, amiloride, chlorothiazide, indapamide, acetazolamide, amiloride, bumetanide, benzthiazide, ethacrynic acid, furosemide, indacrinone, metolazone, spironolactone, triamterene, chlorthalidone and the like or a pharmaceutically acceptable salt thereof.

Representative adrenergic blocking agents include phentolamine, phenoxybenzamine, prazosin, terazosin, tolazine, atenolol, metoprolol, albuterol, nadolol, propranolol, timolol, carteolol and the like or a pharmaceutically acceptable salt thereof.

Representative vasodilators include hydralazine, minoxidil, diazoxide, nitroprusside, flosequinan and the like or a pharmaceutically acceptable salt thereof.

Representative calcium channel blockers include aminone, bencyclane, diltiazem, fendiline, flunarizine, nicardipine, nimodipine, perhexylene, verapamil, gallopamil, nifedipine and the like or a pharmaceutically acceptable salt thereof.

Representative ACE inhibitors include ramipril, aptopril, enalapril, lisinopril, fosinopril, captopril and the like or a pharmaceutically acceptable salt thereof.

Representative potassium channel activators include pinacidil, glibenclamide, glimepiride, diaoxide, cromocalim, and the like or a pharmaceutically acceptable salt thereof.

Representative antiserotoninergic agents include ketanserin and the like or a pharmaceutically acceptable salt thereof.

Representative angiotensin II antagonists include DUP527 and the like or a pharmaceutically acceptable salt thereof.

Representative angiotensin II receptor blockers (angiotensin II receptor antagonists (ARBs)) include losartan, irbesartan, valsartan, omapatrilat, gemopatrilat and the like or a pharmaceutically acceptable salt thereof.

Other representative cardiovascular agents include sympatholytic agents such as methyldopa, clonidine, guanabenz, reserpine and the like or a pharmaceutically acceptable salt thereof.

### Dosage, Host and Safety

The compounds of the present invention are stable and can be used safely. In particular, the compounds of the present invention are useful as renin inhibitors for a variety of subjects (e.g., humans, non-human mammals and non-mammals). The optimal dose may vary depending upon such conditions as, for example, the type of subject, the body weight of the subject, the route of administration, and specific properties of the particular compound being used. In general, the daily dose for oral administration to an adult (body weight of about 60 kg) is about 1 to 1000 mg, about 3 to 300 mg, or about 10 to 200 mg. It will be appreciated that the daily dose can be given in a single administration or in multiple (e.g., 2 or 3) portions a day.

### Compositions Comprising Renin Inhibitors

A wide variety of compositions and administration methods may be used in conjunction with the compounds of the present invention. Such compositions may include, in addition to the compounds of the present invention, conventional pharmaceutical excipients, and other conventional, pharmaceutically inactive agents. Additionally, the compositions may include active agents in addition to the compounds of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The Renin inhibitors and compositions comprising them may be administered or coadministered in any conventional dosage form. Co-administration in the context of this invention is intended to mean the administration of more than one therapeutic agent, one of which includes a Renin inhibitor, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When compounds according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding compounds according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more compounds according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15^{th} Edition, 1975. The composition or formulation to be administered will, in any event, contain a sufficient quantity of an inhibitor of the present invention to reduce Renin activity in vivo, thereby treating the disease state of the subject.

Dosage forms or compositions may optionally comprise one or more compounds according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more Renin inhibitors, optionally 0.1-95%, and optionally 1-95%.

Salts, preferably sodium salts, of the inhibitors may be prepared with carriers that protect the compound against rapid elimination from the body, such as time release formulations or coatings. The formulations may further include other active compounds to obtain desired combinations of properties.

A. Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, compounds according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water-soluble FD and C dyes, mixtures thereof; and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The compounds of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising compounds of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

B. Injectables, Solutions, and Emulsions

The present invention is also directed to compositions designed to administer the compounds of the present invention by parenteral administration, generally characterized by subcutaneous, intramuscular or intravenous injection. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of an inhibitor in the parenteral formulation may be adjusted so that an injection administers a pharmaceutically effective amount sufficient to produce the desired pharmacological effect. The exact concentration of an inhibitor and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is known and practiced in the art.

Injectables may be designed for local and systemic administration. Typically a therapeutically effective dosage is formulated to contain a concentration of at least about 0.1% w/w up to about 90% w/w or more, preferably more than 1% w/w of the Renin inhibitor to the treated tissue(s). The inhibitor may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The Renin inhibitor may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

C. Lyophilized Powders

The compounds of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a Renin inhibitor is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the inhibitor.

D. Topical Administration

The compounds of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The Renin inhibitors may be formulated as aerosols for topical application, such as by inhalation (see, U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The inhibitors may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the Renin inhibitor alone or in combination with other pharmaceutically acceptable excipients can also be administered.

E. Formulations for Other Routes of Administrations

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

F. Examples of Formulations

The following are particular examples of oral, intravenous and tablet formulations that may optionally be used with compounds of the present invention. It is noted that these formulations may be varied depending on the particular compound being used and the indication for which the formulation is going to be used.

ORAL FORMULATION

| | | |
|---|---|---|
| Compound of the Present Invention | 10-100 | mg |
| Citric Acid Monohydrate | 105 | mg |
| Sodium Hydroxide | 18 | mg |
| Flavoring | | |
| Water | q.s. to 100 | mL |

INTRAVENOUS FORMULATION

| Compound of the Present Invention | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

TABLET FORMULATION

| | |
|---|---|
| Compound of the Present Invention | 1% |
| Microcrystalline Cellulose | 73% |
| Stearic Acid | 25% |
| Colloidal Silica | 1%. |

### Kits Comprising Renin Inhibitors

The invention is also directed to kits and other articles of manufacture for treating diseases associated with Renin. It is noted that diseases are intended to cover all conditions for which the Renin possess activity that contributes to the pathology and/or symptomology of the condition.

In one embodiment, a kit is provided that comprises a composition comprising at least one inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Preparation and Biological Testing of Renin Inhibitors

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) and can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this Application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds according to the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| μL (microliters) | Ac (acetyl) |
| atm (atmosphere) | ATP (Adenosine Triphophatase) |
| BOC (tert-butyloxycarbonyl) | BOP (bis(2-oxo-3- |
| | oxazolidinyl)phosphinic |
| | chloride) |
| BSA (Bovine Serum Albumin) | CBZ (benzyloxycarbonyl) |
| CDI (1,1-carbonyldiimidazole) | DCC (dicyclohexylcarbodiimide) |
| DCE (dichloroethane) | DCM (dichloromethane) |
| DMAP (4-dimethylaminopyridine) | DME (1,2-dimethoxyethane) |
| DMF (N,N-dimethylformamide) | DMPU (N,N′- |
| | dimethylpropyleneurea) |
| DMSO (dimethylsulfoxide) | EDCI (ethylcarbodiimide |
| | hydrochloride) |
| EDTA | Et (ethyl) |
| (Ethylenediaminetetraacetic | |
| acid) | |
| Et₂O (diethyl ether) | EtOAc (ethyl acetate) |
| FMOC (9- | g (grams) |
| fluorenylmethoxycarbonyl) | |
| h (hours) | HOAc or AcOH (acetic acid) |
| HOBT (1-hydroxybenzotriazole) | HOSu (N-hydroxysuccinimide) |
| HPLC (high pressure liquid | Hz (Hertz) |
| chromatography) | |
| i.v. (intravenous) | IBCF (isobutyl chloroformate) |
| i-PrOH (isopropanol) | L (liters) |
| M (molar) | mCPBA (meta-chloroperbenzoic |
| | acid) |
| Me (methyl) | MeOH (methanol) |
| mg (milligrams) | MHz (megahertz) |
| min (minutes) | mL (milliliters) |
| mM (millimolar) | mmol (millimoles) |
| mol (moles) | MOPS (Morpholinepropanesulfonic |
| | acid) |
| mp (melting point) | NaOAc (sodium acetate) |
| OMe (methoxy) | psi (pounds per square inch) |
| RP (reverse phase) | RT (ambient temperature) |
| SPA (Scintillation Proximity | TBAF (tetra-n-butylammonium |
| Assay) | fluoride) |
| TBS (t-butyldimethylsilyl) | tBu (tert-butyl) |
| TEA (triethylamine) | TFA (trifluoroacetic acid) |
| TFAA (trifluoroacetic | THF (tetrahydrofuran) |
| anhydride) | |
| TIPS (triisopropylsilyl) | TLC (thin layer chromatography) |
| TMS (trimethylsilyl) | TMSE (2-(trimethylsilyl)ethyl) |
| Tr (retention time) | ° C. (degrees Centigrade) |

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at RT unless otherwise noted.

hu 1H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad). When two rotomers are observed, the combined NMR spectra are presented.

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60F-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosures of all documents cited throughout this application are incorporated herein by reference.

A. Synthetic Schemes for Compounds of the Present Invention

Compounds according to the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

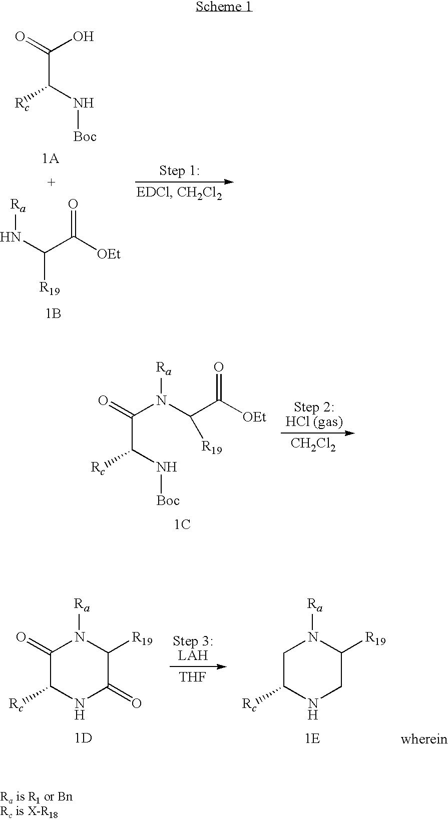

N-Boc protected starting material 1A (1 equiv.) can be coupled to an amino acid derivative 1B (1 equiv.) using ethylcarbodiimide hydrochloride (EDCI) (1.1-1.5 equiv.) and HOBt (1.1-1.5 equiv.) in dichloromethane (0.1-0.5 M) (Step 1). The Boc group can be removed upon bubbling of HCl gas through the cold (0-15° C.) solution of the resulting amide 1C in dichloromethane (0.2-0.5 M). Subsequent cyclization upon removal of the solvent can afford the dioxopiperazine derivative 1D (Step 2), which can be reduced to the corresponding diamine 1E with lithium aluminum hydride (LAH) (2-3 equiv.) in THF (0.2-0.4 M) at reflux for 1-24 h (Step 3).

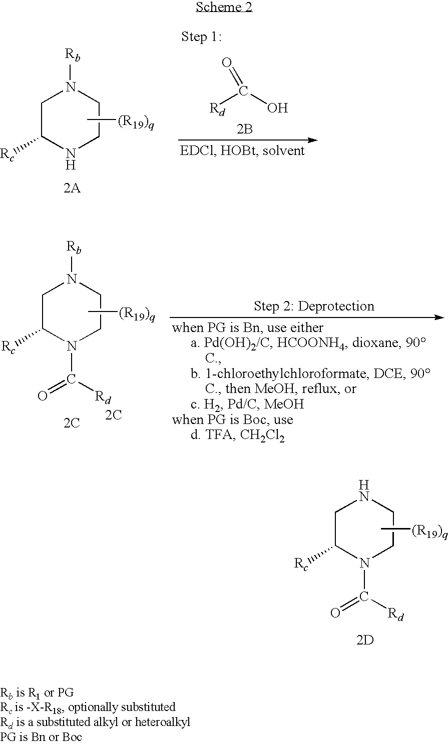

The amine component 2A (1.0 equiv.) can be reacted with a variety of carboxylic acids 2B (1.0-2.0 equiv.) under the standard EDCI (1.0-1.5 equiv.)/HOBt (1.0-1.5 equiv.) conditions in dichloromethane or DMF (0.1-0.5 M) at 0-90° C. (Step 1) forming the corresponding amide 2C. The protective group, if it is benzyl, can be removed (Step 2) using one of the following conditions: (a) Pd(OH)₂/HCOONH₄/dioxane at 90° C. for 0.5-24 h, (b) 1-chloroethylchloroformate, and dichloroethane (DCE), 90° C. for 1-3 h, then MeOH, 20-65° C. for 0.1-2 h, or (c) 30-70 psi H₂, Pd/C, MeOH. The protective group, if it is Boc, can be removed with excess TFA in dichloromethane (room temperature, 0.1-18 h). The crude product 2D can be purified by HPLC (acetonitrile-water, TFA buffered) or flash column chromatography on silica gel.

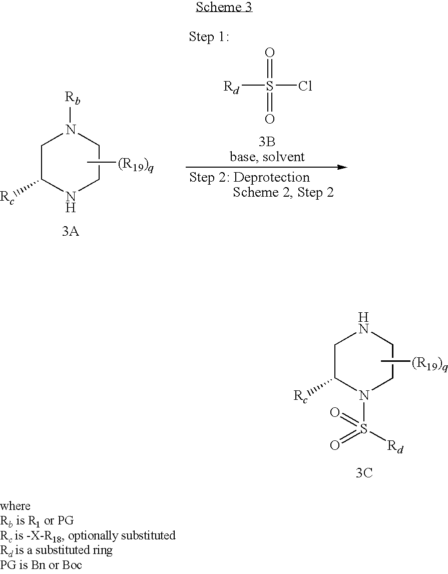

The amine component 3A (1.0 equiv.) can be reacted with a variety of sulfonyl chlorides 3B (1.0-3.0 equiv.) using base (e.g. NEt₃ or pyridine) in an appropriate solvent (e.g. dichloromethane, 0.1-0.5 M). The resulting sulfonamide can be deprotected in a manner identical to that in Scheme 2, Step 2 to afford compound 3C.

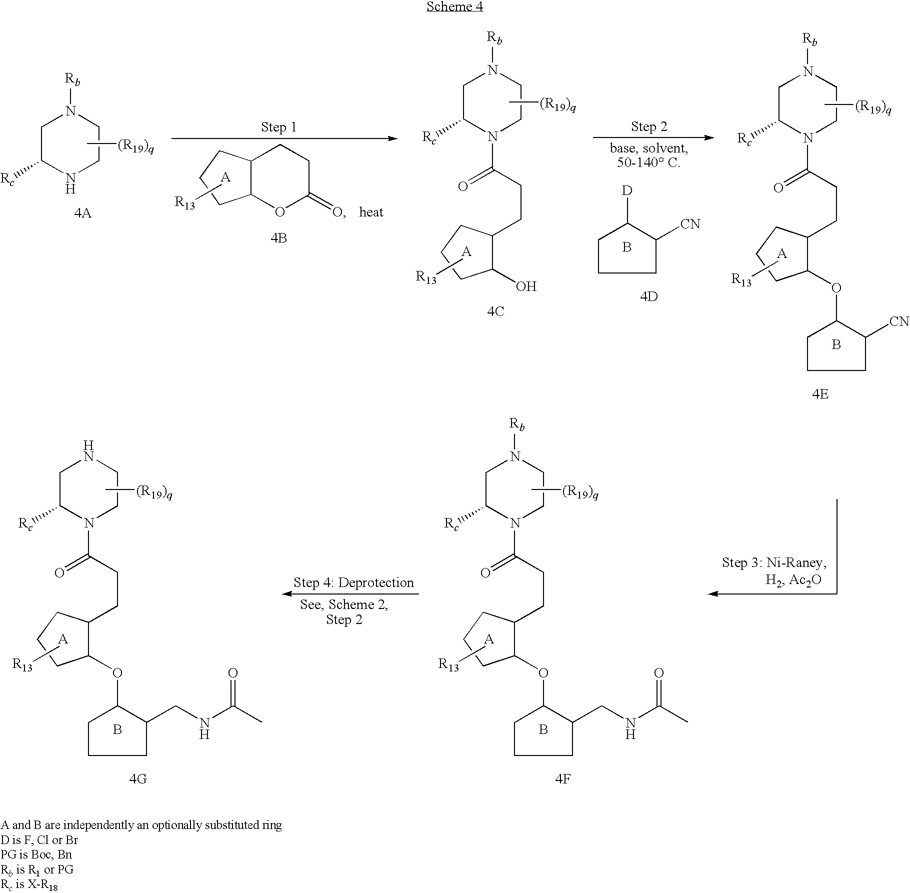

The amine component 4A (1 equiv.) heated with dihydrocoumarine derivatives 4B (1-1.5 equiv.) at 90-120° C. in toluene (0.2-2 M) can provide the corresponding hydroxy-derivative 4C (Step 1). 4C (Step 1) (1 equiv.) can then be used in the nucleophilic displacement of the aryl or heteroaryl halides 4D (1-10 equiv.) using base (e.g. K₂CO₃ or Cs₂CO₃) in acetone or DMF (0.1-1.0 M) upon heating to 50-140° C. for 0.5 h to 3 d (Step 2). The resulting nitrile 4E (1 equiv.) can be reduced to the primary amine and in situ converted to an acetyl-derivative 4F using H₂ (30-100 psi) and Ni-Raney (excess) in Ac₂O (0.05-0.2 M) (Step 3). Finally, deprotection of the secondary amine (Step 4, as described in Scheme 2, Step 2) can afford the target compounds 4G.

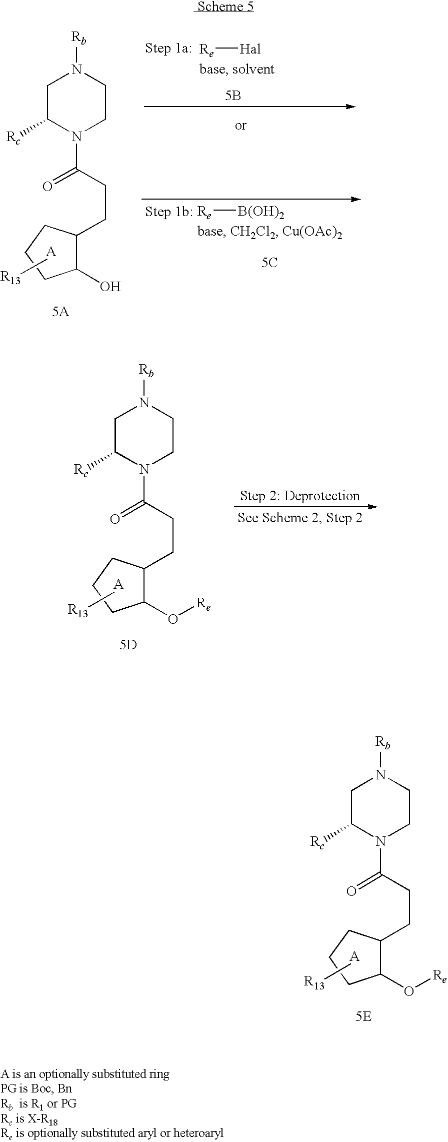

Component 5A (1 equiv.) can be reacted with a substituted or unsubstituted aryl/heteroaryl halide 5B (1-10 equiv.) in the presence of base (e.g. K₂CO₃) in acetone or DMF (0.1-0.5 M) at 50-120° C. (Step 1a) to yield 5D. Alternatively, the same component 5A (1 equiv.) can be coupled to aryl/heteroaryl boronic acids 5C (2-5 equiv.) using copper catalyst (e.g. Cu(OAc)₂, 2-4 equiv.) and pyridine or triethylamine (3-10 equiv.) in dichloromethane (0.1-0.5 M) at room temperature for 1-7 d (Step 1b) to yield 5D. Deprotection (as in Scheme 2, Step 2) of 5D can provide the desired amines 5E (Step 2).

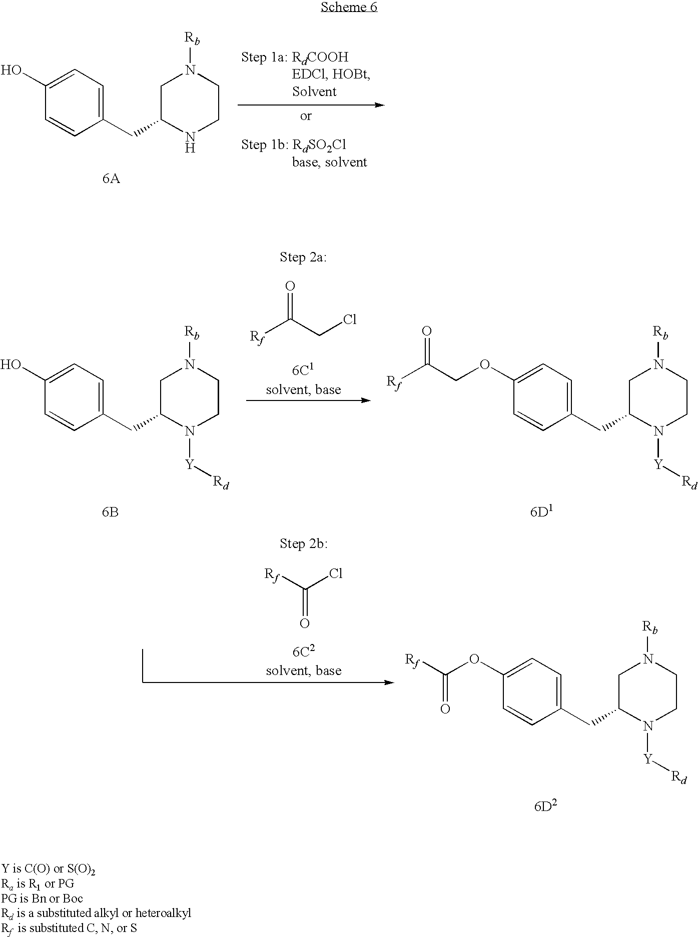

The cyclic substituents on the amine component may be functionalized according to Scheme 6. The amine component 6A can be functionalized as described in Scheme 2 or Scheme 3, respectively, (Step 1a or 1b). The resulting phenol 6B (1 equiv.) can then be reacted with various electrophiles (1-10 equiv.) (Step 2). For example, phenol 6B may react with α-halocarbonyl-containing compounds 6C¹ (3-10 equiv.) using base (e.g. K₂CO₃, 3-5 equiv.) in acetone or DMF (0.1-1.0 M) at 50-130° C. (Step 2a) to afford compounds 6D¹. Also, 6B may react with acid chlorides/carbamoyl chlorides 6C² (1-10 equiv.) in pyridine (0.2-1.2 M) at 0-50° C. (Step 2b) to afford compounds 6D². Compounds 6D¹ and 6D² may be deprotected (when R_{b} is Bn or Boc) as described in Scheme 2, Step 2.

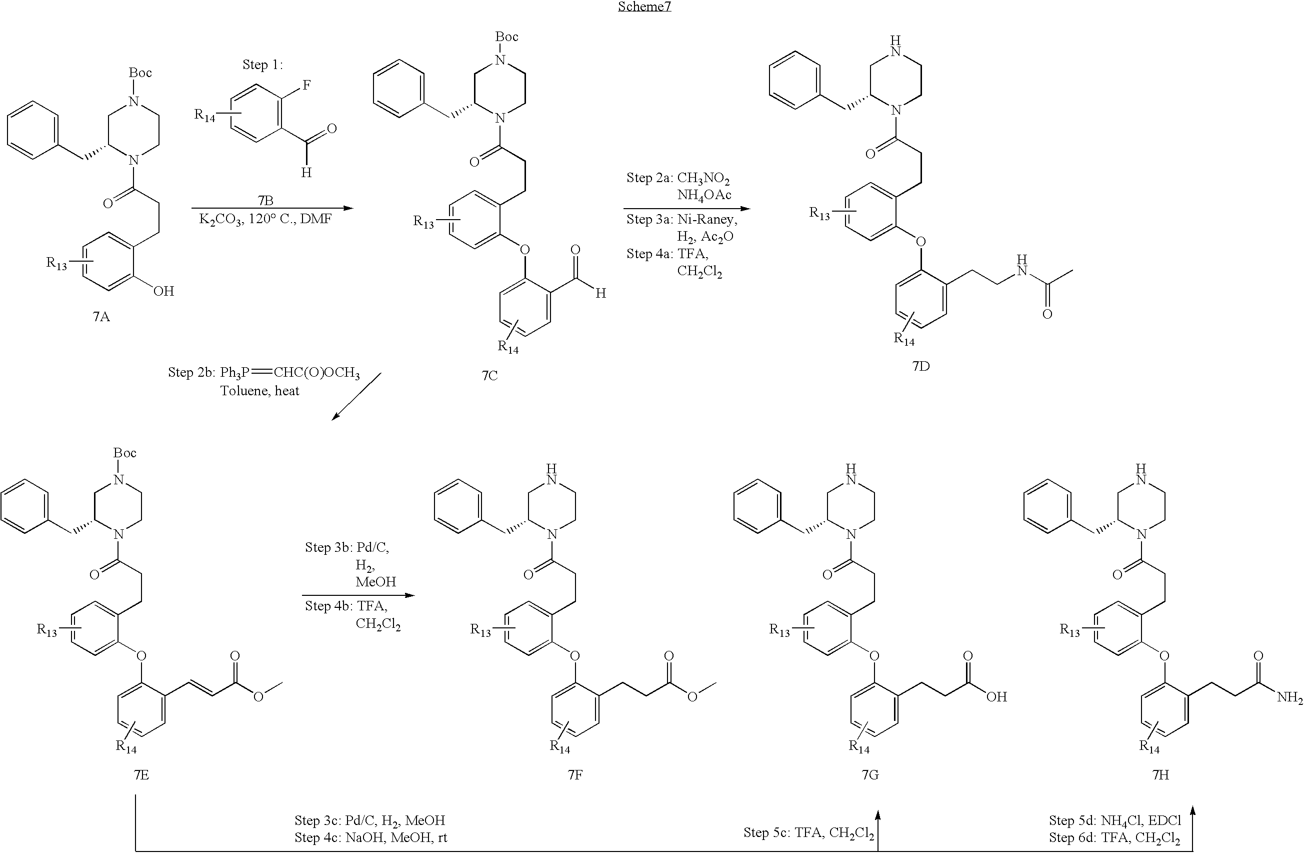

The phenol component 7A (1 equiv.) can be used in the nucleophilic substitution reaction of 2-fluorobenzaldehyde (7B) (1-3 equiv.) using base (e.g. K₂CO₃, 2-5 equiv.) in DMF (0.2-1 M) at elevated temperatures (60-130° C.) (Step 1). The resulting aldehyde 7C can be used for the preparation of several subtypes of piperazine compounds. For example, the aldehyde 7C (1 equiv.) can be converted to the unsaturated nitro-derivative via Henry reaction (Step 2a), using nitromethane (20-100 equiv.) and ammonium acetate (1.1-2.0 equiv.) at 60-100° C. for 0.3-3 h. The resulting nitro-compound (1 equiv.) can then be reduced to the amine and converted to the acyl derivative (Step 3a, as described in Scheme 4, Step 3), which is then deprotected (Step 4a, as described in Scheme 2, Step 2d) to afford the desired compound 7D. Alternatively, aldehyde 7C (1 equiv.) can be reacted with methyl (triphenylphosphoranylidene) acetate (1.1-1.5 equiv.) in toluene (0.5-1.0 M) at elevated temperature (80-110° C.) for 1-24 h (Step 2b). The resulting alkene 7E (1 equiv.) can be hydrogenated (Pd/C, 50-70 psi H₂) in alcohol (e.g. MeOH, 0.1-0.3 M) to give the corresponding saturated ester (Step 3b). The ester can then either be: (a) deprotected (Step 4b, as described in Scheme 2, Step 2d) to give the desired ester containing compound 7F; (b) saponified with excess aq. NaOH (1 M) in MeOH (0.1-0.3 M) (Step 4c) and deprotected to give the desired acid containing compound 7G (Step 5c, as described in Scheme 2, Step 2d); or (c) saponified (Step 4c), and then converted to the corresponding amide using NH₄Cl (1.1-1.5 equiv.), EDCI (1.1-1.5 equiv.), HOBt (1.1-1.5 equiv.) and TEA (1.0-3.0 equiv.) in dichloromethane (0.1-0.3 M) (Step 5d) and deprotected to afford amide 7H (Step 6d, as described in Scheme 2, Step 2).

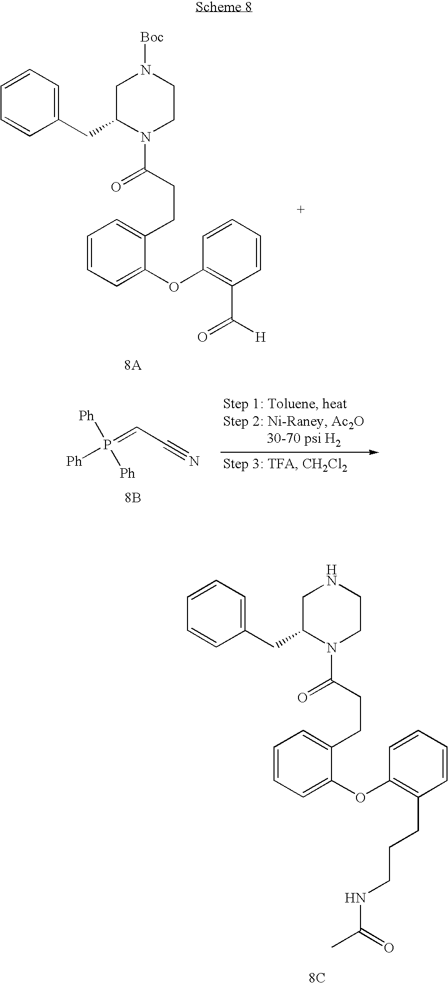

The aldehyde 8A (1 equiv.) can be converted to the unsaturated nitrile derivative via a Wittig reaction (Step 1), using (triphenylphosphoranylidene)acetonitrile (8B) (1.0-1.5 equiv.) in toluene upon heating (80-110° C.). The resulting nitrile (1 equiv.) can then be reduced to the amine and converted to the acyl derivative in situ (Step 2, as described in Scheme 4, Step 3). The desired final compound 8C can be obtained via deprotection (Step 3, as described in Scheme 2, Step 2d).

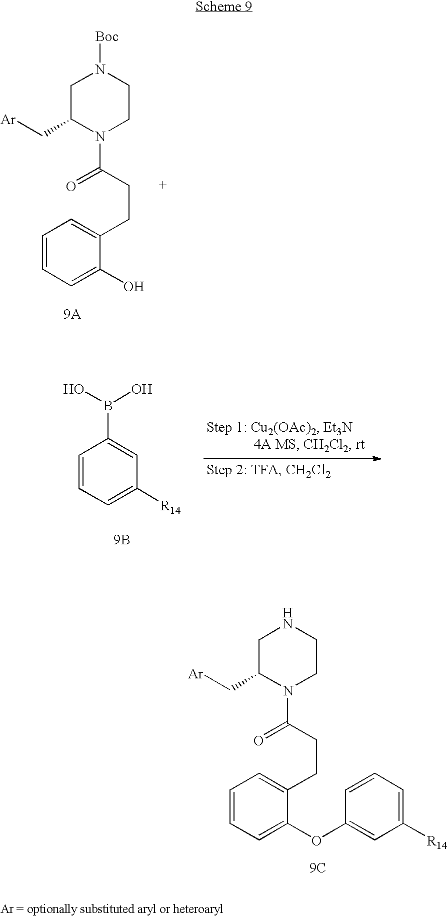

The phenol component 9A (1 equiv.) can be coupled to various boronic acids 9B (2-4 equiv.) using Cu(OAc)₂ (3-10 equiv.) in the presence of base (TEA or pyridine, 3-10 equiv.) and 4 A molecular sieves (4A MS) in dichloromethane (0.1-0.3 M) at room temperature for 1-7 d (Step 1). Deprotection can then provide the desired amines 9C (Step 2, as described in Scheme 2, Step 2d).

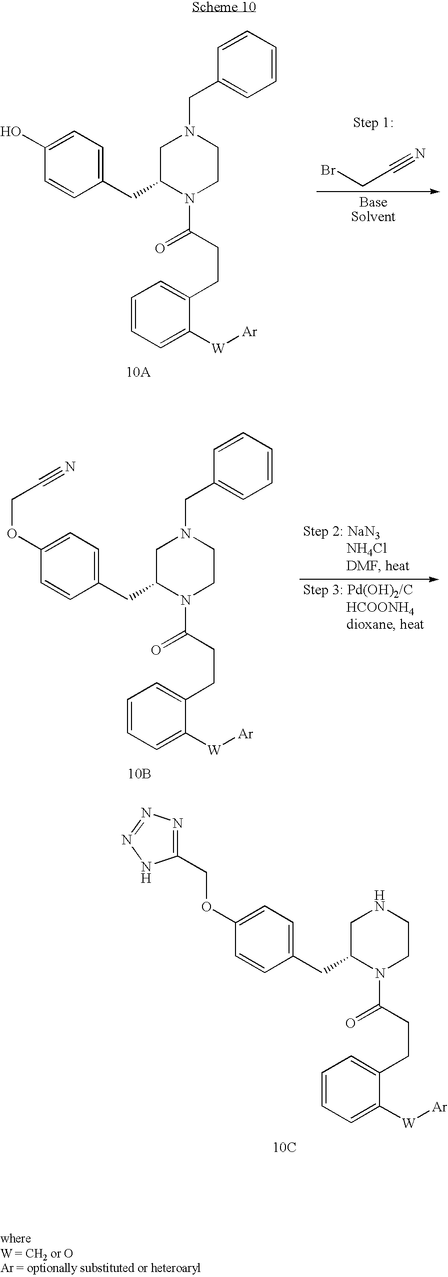

The phenol component 10A (1 equiv.) can be treated with bromoacetonitrile (1.0-2.0 equiv.) in the presence of base (e.g. K₂CO₃, 3-5 equiv.) in acetone (0.5 M) at 30-60° C. for 0.5-3 d (Step 1). The resulting nitrile 10B (1 equiv.) can then be converted to the tetrazole moiety using NaN₃ (1 equiv.) and ammonium chloride (1 equiv.) in DMF (0.3 M) upon heating at 80-130° C. (Step 2). Deprotection (Step 3, as described in Scheme 2, Step 2a) can afford the desired compound 10C.

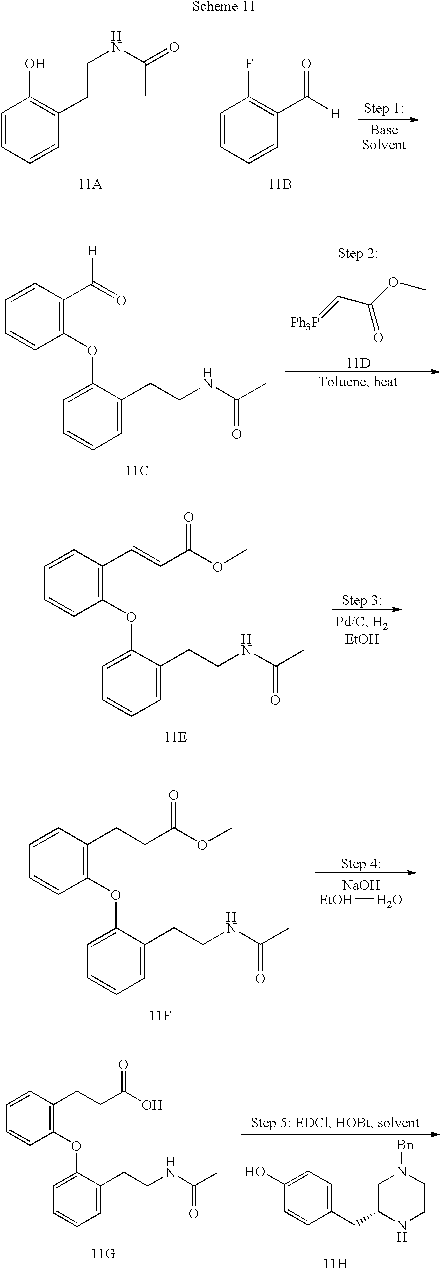

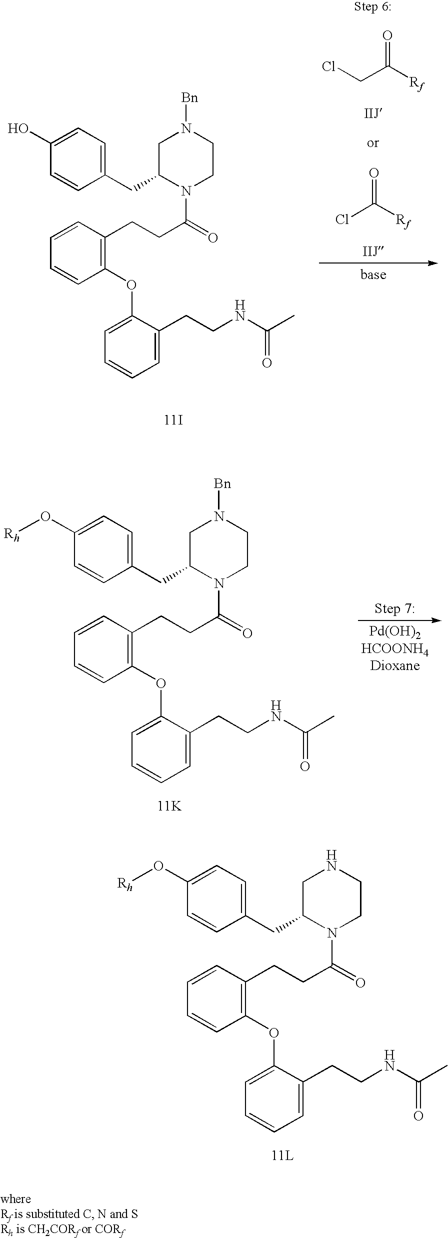

The phenol component 11A (1 equiv.) can be used in the nucleophilic substitution reaction of 2-fluorobenzaldehyde (111B) (1-3 equiv.) using base (e.g. K₂CO₃, 2-5 equiv.) in DMF (0.2-1 M) at elevated temperatures (60-130° C.) (Step 1). The resulting aldehyde 11C (1 equiv.) can be reacted with the methyl (triphenylphosphoranylidene) acetate (11D) (1.1-1.5 equiv.) in toluene (0.3-1.0 M) at elevated temperature (80-110° C.) (Step 2). The resulting alkene 11E (1 equiv.) can be hydrogenated (Pd/C, 50-70 psi H₂) in EtOH (0.1-0.3 M) to give the corresponding saturated ester 11F (Step 3). The ester can then be saponified with an excess of 1M aq. NaOH in EtOH (0.1-0.3 M) to the carboxylic acid 11G (Step 4). The acid 11G can be coupled to the amine component 11H (Step 5, as described in Scheme 2, Step 1). The resulting phenol 11I can then be derivatized with various electrophiles (e.g. 11J′ and 11J″) (Step 6, as described in Scheme 6, Steps 2a and 2b) to form 11K and deprotected to form 11L (Step 7, as described in Scheme 2, Step 2a).

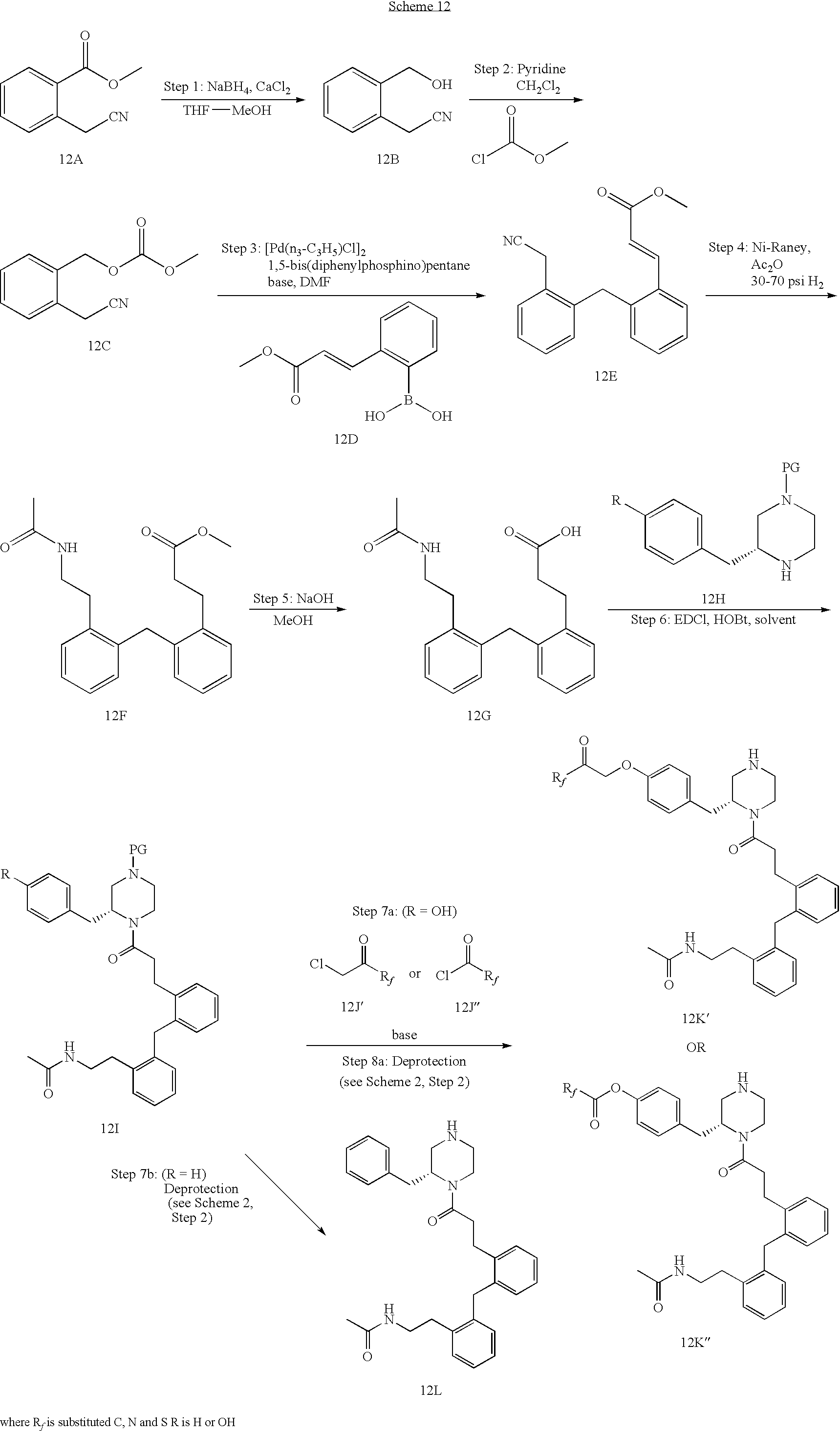

In Step 1, the starting ester 12A (1 equiv.) can be reduced with NaBH₄ (10 equiv.) in the presence of CaCl₂ (2 equiv.) in THF (0.05-0.15 M). The resulting alcohol 12B (1 equiv.) can be converted to the carbonate 12C with methylchloroformate (1 equiv.) and pyridine (2-5 equiv.) in dichloromethane (0.1-0.5 M) at 0-30° C. (Step 2). The carbonate 12C (1 equiv.) can then be used in the Suzuki coupling (Step 3) with boronic acids 12D (0.7-3 equiv.) using allylpalladium (II) chloride dimer (0.02-0.2 equiv.), 1,5-bis-(diphenylphosphino)pentane (0.04-0.4 equiv.), and a base (e.g. K₂CO₃, 2.2 equiv.) in DMF (1 M). The resulting nitrile 12E can then be reduced to the amine and converted in situ to the acetylamine 12F (Step 4, as described in Scheme 4, Step 3). The ester 12F (1 equiv.) can be saponified with a base (e.g. excess 1M aq. NaOH) in MeOH (0.1-0.3 M) (Step 5). The resulting acid 12G (1 equiv.) can be reacted with the piperazine component 12H (1-1.5 equiv.) using standard coupling conditions affording the corresponding N-protected amine (Step 6, as described in Scheme 2, step 1). If R═H, the Boc or Bn group can be removed to afford the amine 12L (Step 7b, as described in Scheme 2, Step 2). When R═OH, the N-protected amine 12I may be functionalized with various electrophiles, e.g., with compounds 12J′ or 12J″, (Step 7a, as described in Scheme 6, Steps 2a or 2b) and then deprotected to form the corresponding amines, 12K′ or 12K″, respectively (Step 8a, as described in Scheme 2, Step 2).

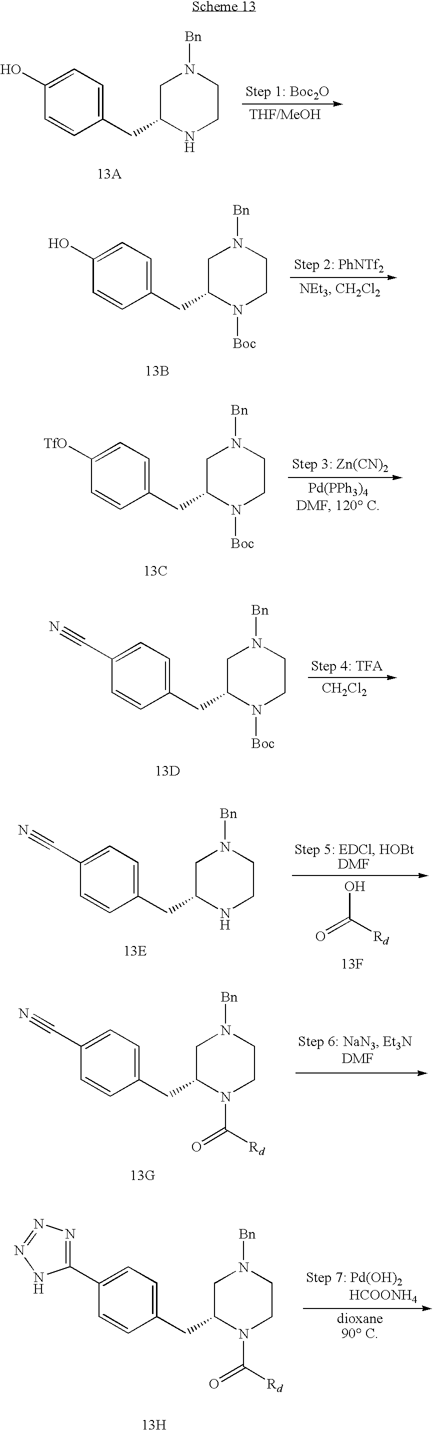

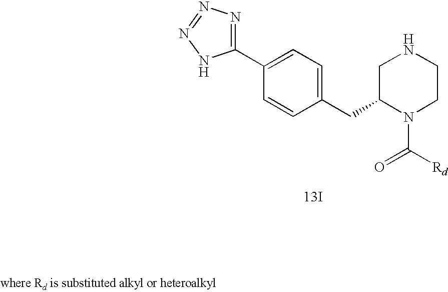

The Bn-protected piperazine 13A can be further protected with Boc-group to afford compound 13B using Boc₂O in THF/MeOH mixture at 0° C. to room temperature for 1-24 h (Step 1). 13B can then be converted to the corresponding triflate 13C with PhNTf₂ (1.0-1.2 equiv.) and Et₃N (2-3 equiv.) in dichloromethane (0.05-0.5M) (Step 2). The triflate 13C (1 equiv.) can then be reacted with zinc (II) cyanide (2 equiv.) in the presence of Pd(PPh₃)₄ (0.05-0.2 equiv.) in DMF (0.05-0.5 M) at 100-140° C. for 1-12 h to afford the nitrile 13D (Step 3). Boc group can be removed under the standard TFA/CH₂Cl₂ conditions as described in Scheme 2, Step 2d (Step 4) and the resulting amine 13E (1 equiv.) can be coupled to a variety of carboxylic acids (13F, 0.5-3 equiv.) using EDCI (1-2 equiv.) and HOBt (1-2 equiv.) in dichloromethane or DMF (0.05-1M) to afford amides 13G (Step 5). 13G can then be heated with NaN₃ (1-2 equiv.) and Et₃N—HCl (1-2 equiv.) in DMF (0.1-1M) at 80-120° C. for 1-3 days to form tetrazoles 13H (Step 6). Deprotection of the amino moiety can finally serve the desired piperazines 131 as described in Scheme 2, Step 2a (Step 7).

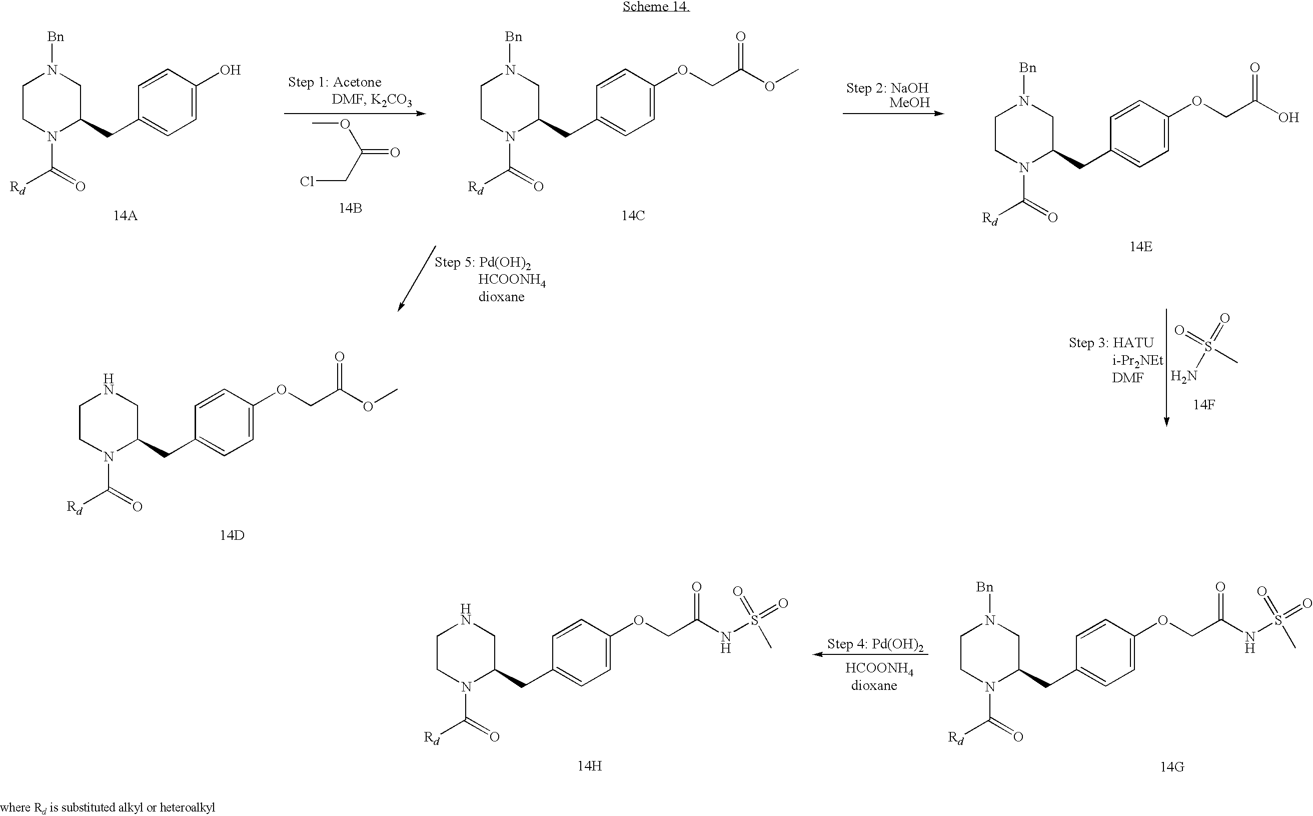

Phenol 14A (obtained as described in Scheme 2, Step 1) can be alkylated with chloroacetate derivatives, e.g., methyl chloroacetate (14B), in acetone and/or DMF (0.05-0.2M) with base (e.g. K₂CO₃, 3-5 equiv.) upon heating at 60° C. for 3 h to 3d. The resulting ester 14C can then be saponified with base (e.g. 1M NaOH) in alcohol (e.g. MeOH, 0.05-0.2M) at room temperature to 100° C. over 1-24 h to afford the corresponding carboxylic acid 14E. The acid can be coupled with sulfonamide 14F (1-5 equiv.) employing HATU (1-2 equiv.) and Hunig's base (3-5 equiv.) in DMF (0.05-0.2 M) to afford 14G. The benzyl group can then be removed as described in Scheme 2, Step 2a to afford the desired product 14H.

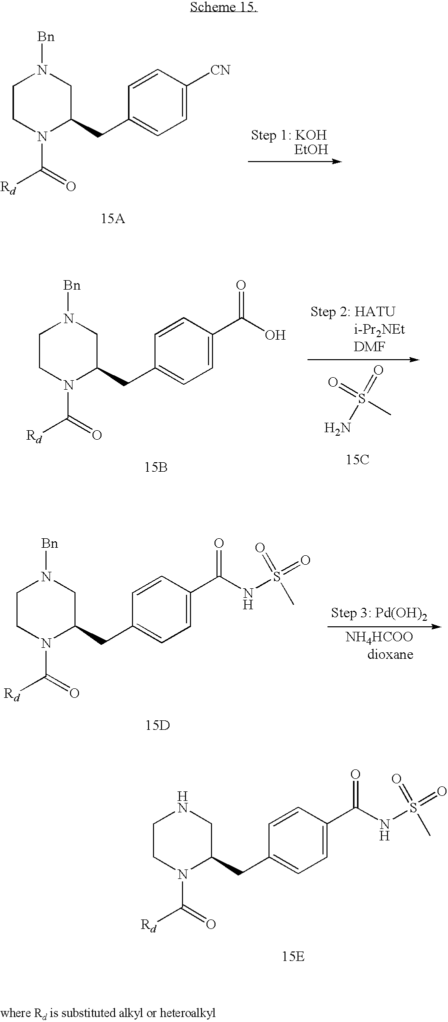

The cyano-derivative 15A (obtained as described in Scheme 13, Steps 1-5) can be converted to the carboxylic acid 15B upon heating with aqueous base, e.g. KOH (2-5 equiv.), in EtOH (0.5-0.2M) for 5-24 h. 15B can then be coupled to methanesulfonamide (15C) as described in Scheme 14 Step 3 with heating at 70-100° C. to afford 15D. The benzyl group can then be removed as described in Scheme 2, Step 2a to afford 15E.

Chiral components can be separated and purified using any of a variety of techniques known to those skilled in the art. For example, chiral components can be purified using supercritical fluid chromatography (SFC). In one particular variation, chiral analytical SFC/MS analyses are conducted using a Berger analytical SFC system (AutoChem, Newark, Del.) which consists of a Berger SFC dual pump fluid control module with a Berger FCM 1100/1200 supercritical fluid pump and FCM 1200 modifier fluid pump, a Berger TCM 2000 oven, and an Alcott 718 autosampler. The integrated system can be controlled by BI-SFC Chemstation software version 3.4. Detection can be accomplished with a Watrers ZQ 2000 detector operated in positive mode with an ESI interface and a scan range from 200-800 Da with 0.5 second per scan. Chromatographic separations can be performed on a ChiralPak AD-H, ChiralPak AS-H, ChiralCel OD-H, or ChiralCel OJ-H column (5μ, 4.6×250 mm; Chiral Technologies, Inc. West Chester, Pa.) with 10 to 40% methanol as the modifier and with or without ammonium acetate (10 mM). Any of a variety of flow rates can be utilized including, for example, 1.5 or 3.5 mL/min with an inlet pressure set at 100 bar. Additionally, a variety of sample injection conditions can be used including, for example, sample injections of either 5 or 10 L in methanol at 0.1 mg/mL in concentration.

In another variation, preparative chiral separations are performed using a Berger MultiGram II SFC purification system. For example, samples can be loaded onto a ChiralPak AD column (21×250 mm, 10μ). In particular variations, the flow rate for separation can be 70 mL/min, the injection volume up to 2 mL, and the inlet pressure set at 130 bar. Stacked injections can be applied to increase the efficiency.

In each of the above reaction procedures or schemes, the various substituents may be selected from among the various substituents otherwise taught herein.

Descriptions of the syntheses of particular compounds according to the present invention based on the above reaction scheme are set forth herein.

The present invention is further exemplified, but not limited, by examples provided below that describe the synthesis of particular compounds according to the invention.

B. Biological Testing

The activity of compounds as Renin inhibitors may be assayed in vitro, in vivo or in a cell line. Example D below provides an in vitro enzymatic activity assay for activity against Renin.

Test compounds in varying concentrations may be reacted with recombinant human renin in the presence of substrate, e.g., QXL520-γ-Abu-Ile-His-Pro-Phe-His-Leu-Val-Ile-His-Thr-Lys (HiLyteFluo488)-Arg-OH (Anaspec, San Jose, Calif.). The reaction can be followed kinetically using fluorescence (excitation λ=485 nm; emission λ=538 nm). Inhibition constants (IC₅₀) may be calculated by non-linear curve fitting of the compound concentrations and fluorescence intensities to the standard IC₅₀ equation. IC₅₀ values for selected compounds of the present invention are given in Table 1.

The present invention is further exemplified, but not limited by, the following examples that describe the synthesis and testing of particular compounds according to the invention.

### EXAMPLES

### Example 1

### (R)-1-(2-Benzylpiperazin-1-yl)-3-phenylpropan-1-one

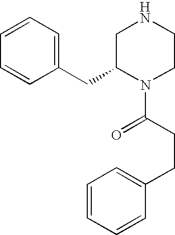

The title compound was prepared as described in Scheme 2. Specifically, 3-phenylpropionic acid (160 mg, 1.07 mmol) and (R)-1,3-dibenzylpiperazine (267 mg, 1.00 mmol) were dissolved in dichloromethane and EDCI (230 mg, 1.20 mmol) was added followed by HOBt (162 mg, 1.05 mmol). The reaction mixture was stirred at room temperature for 2 h, diluted with ethyl ether (10 mL), washed with water (5 mL), NaHCO₃ (sat. aq., 2×10 mL), dried (MgSO₄), filtered and concentrated in vacuo to afford 0.378 g of crude yellow oil. It was dissolved in MeOH (10 mL) and Pd/C (10% w/w, 92 mg) was added. The reaction mixture was hydrogenated at 70 psi of H₂ for 3d, filtered through a short plug of celite and concentrated in vacuo. The residue was purified by HPLC (25-40% acetonitrile in water, TFA buffered) to afford the title compound as a white semisolid (75.3 mg, 24%). ¹H NMR (due to a ˜1:1 mixture of the tertiary amide rotamers, combined NMR spectral data for both rotamers are reported herein, leading to 2× hydrogen count) (400 MHz, DMSO-D6) δ ppm 1.80 (s, 1H) 2.30-2.46 (m, 7H) 2.54-2.75 (m, 8H) 2.79-2.90 (m, 5H) 3.00-3.18 (m, 3H) 3.56 (d, J=12.38 Hz, 1H) 3.84-3.92 (m, 1H) 4.18-4.25 (m, 1H) 4.43-4.56 (m, 1H) 7.00 (d, J=7.07 Hz, 2H) 7.10-7.28 (m, 18H). ESI-MS: m/z 309.4 (M+H)⁺.

### Example 2

### (R)-1-(2-Benzylpiperazin-1-yl)-3,3-diphenylpropan-1-one

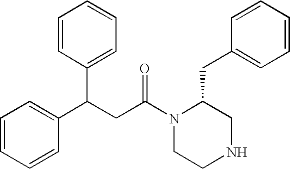

The title compound was prepared as described for Example 1. ¹H NMR (due to a ˜1:1 mixture of the tertiary amide rotamers, combined NMR spectral data for both rotamers are reported herein, leading to 2× hydrogen count) (400 MHz, DMSO-D6) δ ppm 2.14-2.45 (m, 8H) 2.51-3.17 (m, 12H) 3.71-4.47 (m, 6H) 6.88-6.92 (m, 2H) 7.06-7.32 (m, 28H); ESI-MS: m/z 385.5 (M+H)⁺.

### Example 3

### (R)-1-(2-Benzylpiperazin-1-yl)-2-(4′-fluorobiphenyl-2-yl)ethanone

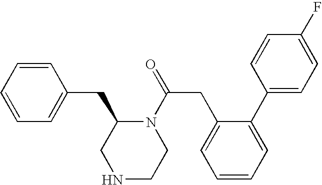

The title compound was prepared as described in Scheme 2. Specifically, 2-(4′-fluorobiphenyl-2-yl)acetic acid (246.0 mg, 1.07 mmol) and (R)-1,3-dibenzylpiperazine (267 mg, 1.00 mmol) were dissolved in dichloromethane (6 mL) and EDCI (230 mg, 1.20 mmol) was added followed by HOBt (162 mg, 1.05 mmol). The reaction mixture was stirred at room temperature for 16 h, diluted with dichloromethane (10 mL), washed with water (5 mL), NaHCO₃ (sat. aq. 2×10 mL), dried (MgSO₄), filtered and concentrated in vacuo to afford 0.38 g of crude brown oil. It was dissolved in dioxane (5 mL) and ammonium formate (200.0 mg, 3.18 mmol) was added followed by Pd(OH)₂/C (20% w/w, 114.0 mg). The reaction was sealed and heated to 80° C. for 1 hr. The crude mixture was then cooled to room temperature and diluted with dichloromethane (10 mL). The solution was filtered through a plug of celite and concentrated to an oil. The residue was purified by HPLC (30-60% acetonitrile in water, TFA buffered) to afford the title compound as a white semisolid (28.2 mg, 7.2%). ¹H NMR (due to a ˜1:1 mixture of the tertiary amide rotamers, combined NMR spectral data for both rotamers are reported herein, leading to 2× hydrogen count) (400 MHz, DMSO-D6) δ, ppm: 2.26-2.45 (m, 4H), 2.57-3.11 (m, 12H), 3.25 (d, J=17.33 Hz, 1H), 3.45-3.55 (m, 3H), 4.15 (d, J=11.58 Hz, 1H), 4.48 (bs, 1H), 6.90-7.28 (m, 26H). ESI-MS: m/z 389.4 (M+H)⁺.

### Example 4

### (R)-1-(2-Benzylpiperazin-1-yl)-2-(2-bromophenoxy)ethanone

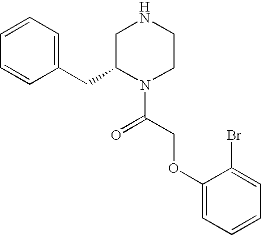

The title compound was prepared as described in Scheme 2 via the following steps:

### Step A: (R)-2-(2-Bromophenoxy)-1-(2,4-dibenzylpiperazin-1-yl)ethanone

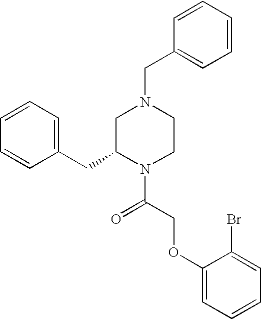

2-(2-Bromophenoxy)acetic acid (247 mg, 1.07 mmol) and (R)-1,3-dibenzylpiperazine (267 mg, 1.00 mmol) were dissolved in dichloromethane and EDCI (230 mg, 1.20 mmol) was added followed by HOBt (162 mg, 1.05 mmol). The reaction mixture was stirred at room temperature for 2 h, diluted with ethyl ether (10 mL), washed with water (5 mL), NaHCO₃ (sat. aq. 2×10 mL), dried (MgSO₄), filtered and concentrated in vacuo to afford the crude yellow oil (0.415 g, 87%), which was used in the next step without further purification. ESI-MS: m/z 479.3 (M+H)⁺.

### Step B: (R)-1-(2-Benzylpiperazin-1-yl)-2-(2-bromophenoxy)ethanone

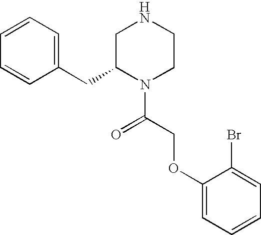

(R)-2-(2-Bromophenoxy)-1-(2,4-dibenzylpiperazin-1-yl)ethanone (0.415 g, 0.866 mmol) was dissolved in dichloroethane (3 mL) and cooled to 0° C. To this solution was added 1-chloroethyl chloroformate (0.61 mL, 5.6 mmol). The reaction mixture was allowed to warm to room temperature and was then heated at 80° C. for 16 hand at 110° C. for an additional 3 h. The reaction mixture was then allowed to cool to room temperature and methanol (10 mL) was added and the solution was stirred at room temperature for thirty minutes. The crude reaction mixture was concentrated in vacuo and the dark brown residue was purified by HPLC (30-60% acetonitrile in water, TFA buffered) to afford the title compound as a white semisolid (17.0 mg, 5.1%). ESI-MS: m/z 389.3 (M+H)⁺.

### Example 5

### (R)-1-(2-Benzylpiperazin-1-yl)-4-phenylbutane-1,4-dione

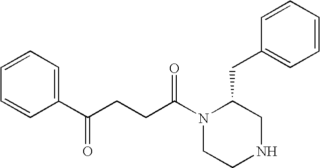

The title compound was prepared as described for Example 4. ESI-MS: m/z 337.4 (M+H)⁺.

### Example 6

### 1-((R)-2-Benzylpiperazin-1-yl)-2,3-diphenylpropan-1-one

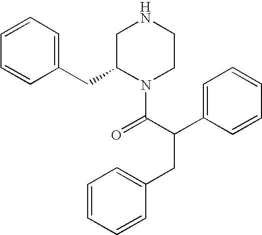

The title compound was prepared as described for Example 3. ESI-MS: m/z 385.4 (M+H)⁺.

### Example 7

### (R)-1-(2-Benzylpiperazin-1-yl)-3-(2-phenoxyphenyl)propan-1-one

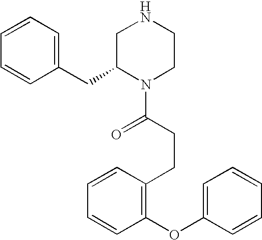

The title compound was prepared as described for Example 3. ESI-MS: m/z 401.4 (M+H)⁺.

### Example 8

### (R)-1-(2-Benzylpiperazin-1-yl)-2-(2-phenoxyphenyl)ethanone

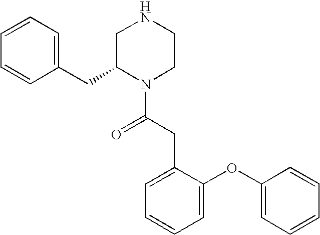

The title compound was prepared as described for Example 3. ESI-MS: m/z 387.4 (M+H)⁺.

### Example 9

### 1-((R)-2-Benzylpiperazin-1-yl)-2-(2′-methoxybiphenyl-2-yl)ethanone

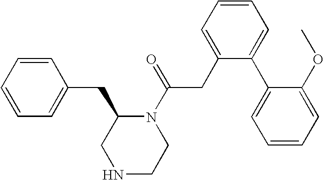

The title compound was prepared as described for Example 3. ESI-MS: m/z 401.4 (M+H)⁺.

### Example 10

### (R)—N-Benzhydryl-2-benzylpiperazine-1-carboxamide

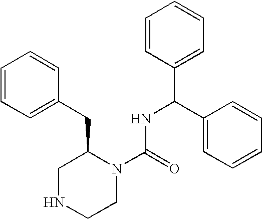

(R)-1,3-dibenzylpiperazine (200 mg, 0.751 mmol) was dissolved in dichloromethane (1.5 mL). To the stirred solution was added diphenylmethyl isocyanate (0.14 mL, 0.75 mmol) via syringe. The reaction mixture was allowed to stir at room temperature for 3 h. The reaction mixture was diluted with dichloromethane (10 mL) and washed with water (2×10 mL) and brine (10 mL). The organic layer was separated, dried (Na₂SO₄), and concentrated to yield an amorphous solid (0.315 g). It was dissolved in MeOH (10 mL) and Pd/C (10%, 92 mg) was added. The reaction mixture was hydrogenated at 70 psi of H₂ for 3d, filtered through a short plug of celite and concentrated in vacuo. The residue was purified by HPLC (25-40% acetonitrile in water, TFA buffered) to afford the title compound as a white semisolid (25 mg, 8.6%); ESI-MS: m/z 386.4 (M+H)⁺.

### Example 11

### 1-((R)-2-Benzylpiperazin-1-yl)-2-(2′-chlorobiphenyl-2-yl)ethanone

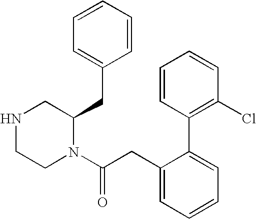

The title compound was prepared as described for Example 4. ESI-MS: m/z 405.3 (M+H)⁺.

### Example 12

### (R)-2-Benzyl-1-(phenethylsulfonyl)piperazine

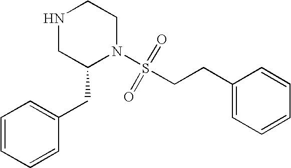

The title compound was prepared as described in Scheme 3. Specifically, (R)-1,3-dibenzylpiperazine (267 mg, 1.00 mmol) was dissolved in dichloromethane (5 mL) and TEA (1 mL, 7.17 mmol) was added, followed by 2-phenethylsulfonyl chloride (307 mg, 1.50 mmol). The reaction mixture was stirred at room temperature for 10 min, concentrated in vacuo and dissolved in dioxane (10 mL). Ammonium formate (0.500 g, 7.93 mmol) and Pd(OH)₂/C (10%, 150 mg) were added. The vial was sealed and heated at 85° C. for 0.5 h. The reaction mixture was filtered through celite, concentrated in vacuo and the crude product was purified by HPLC (20-35% acetonitrile in water, buffered by 0.05% TFA) to afford the title compound as a white solid (30.5 mg, 59%). ¹H NMR (400 MHz, CHLOROFORM-D) δ ppm 2.66 (ddd, J=13.33, 11.18, 5.31 Hz, 1H) 2.77-2.98 (m, 6H) 3.03 (d, J=11.87 Hz, 1H) 3.14 (d, J=7.83 Hz, 2H) 3.35 (td, J=12.82, 3.16 Hz, 1H) 3.57-3.62 (m, 1H) 4.07-4.15 (m, 1H) 7.05-7.08 (m, 2H) 7.11-7.17 (m, 1H) 7.19-7.30 (m, 7H); ESI-MS: m/z 345.4 (M+H)⁺.

### Example 13

### (R)-2-Benzyl-1-(2,2-diphenylethylsulfonyl)piperazine

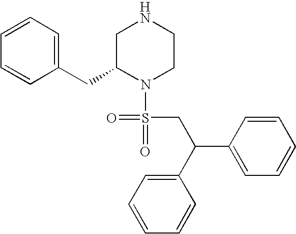

The title compound was prepared as described for Example 12. ESI-MS: m/z 421.4 (M+H)⁺.

### Example 14

### 2-(3-((R)-2-Benzylpiperazin-1-yl)-3-oxo-1-p-tolylpropyl)isoindolin-1-one

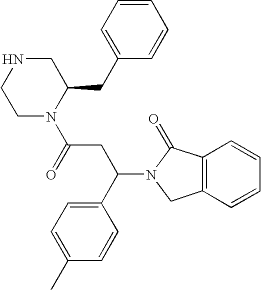

The title compound was prepared as described for Example 3. ESI-MS: m/z 454.4 (M+H)⁺.

### Example 15

### (R)-1-(2-Benzylpiperazin-1-yl)-2-(naphthalen-1-yloxy)ethanone

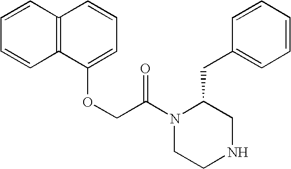

The title compound was prepared as described for Example 3. ESI-MS: m/z 361.4 (M+H)⁺.

### Example 16

### 1-((R)-2-Benzylpiperazin-1-yl)-3-(furan-2-yl)-4-phenylbutan-1-one

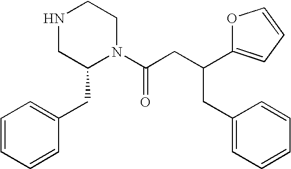

The title compound was prepared as described for Example 3. ESI-MS: m/z 389.4 (M+H)⁺.

### Example 17

### (R)-1-((R)-2-Benzylpiperazin-1-yl)-3-phenylbutan-1-one

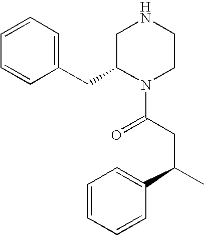

The title compound was prepared as described for Example 3. ESI-MS: m/z 323.4 (M+H)⁺.

### Example 18

### (S)-1-((R)-2-Benzylpiperazin-1-yl)-3-phenylbutan-1-one

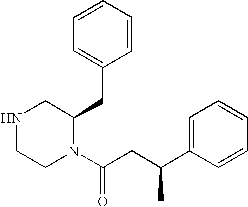

The title compound was prepared as described for Example 3. ESI-MS: m/z 323.4 (M+H)⁺.

### Example 19

### (R)-1-(2-Benzylpiperazin-1-yl)-4-phenylbutan-1-one

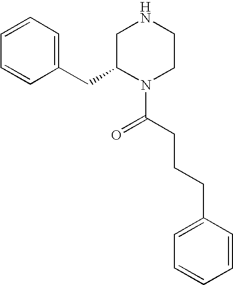

The title compound was prepared as described for Example 3. ESI-MS: m/z 323.4 (M+H)⁺.

### Example 20

### 1-((R)-2-Benzylpiperazin-1-yl)-2-(2,2-dimethyl-4-phenyltetrahydro-2H-pyran-4-yl)ethanone

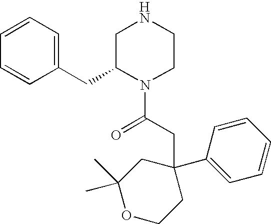

The title compound was prepared as described for Example 3. ESI-MS: m/z 407.4 (M+H)⁺.

### Example 21

### (R)-2-(3-(2-Benzylpiperazin-1-yl)-3-oxopropyl)phenyl benzoate

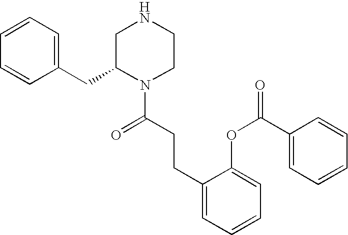

The title compound was prepared as described in Scheme 2. Specifically, 3-(2-(Benzoyloxy)phenyl)propanoic acid (290 mg, 1.07 mmol) and (R)-tert-butyl 3-benzylpiperazine-1-carboxylate (276 mg, 1.00 mmol) were dissolved in dichloromethane (5 mL) and EDCI (230 mg, 1.20 mmol) was added followed by HOBt (162 mg, 1.05 mmol). The reaction mixture was stirred at room temperature for 16 h, diluted with dichloromethane (10 mL), washed with water (5 mL), NaHCO₃ (sat. aq., 2×10 mL), dried (MgSO₄), filtered and concentrated in vacuo to afford 0.315 g of crude brown oil. It was dissolved in dichloromethane (3 mL) and trifluoroacetic acid (2 mL) was added. The reaction mixture was allowed to stir at rt for 2 h and was concentrated in vacuo to an orange oil. The residue was purified by HPLC (30-60% acetonitrile in water, TFA buffered) to afford the title compound as a white semisolid (25.0 mg, 5.8%). ESI-MS: m/z 429.3 (M+H)⁺.

### Example 22

### (R)-1-(2-Benzylpiperazin-1-yl)-3-(2-(4-chlorophenoxy)phenyl)propan-1-one

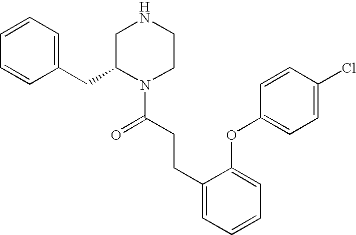

The title compound was prepared as described for Example 21. ESI-MS: m/z 435.4 (M+H)⁺.

### Example 23

### (R)-1-(2-benzylpiperazin-1-yl)-3-(2-(m-tolyloxy)phenyl)propan-1-one

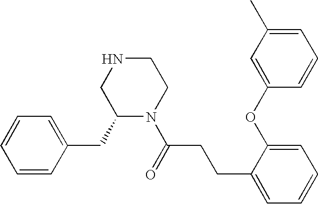

The title compound was prepared as described for Example 3. ESI-MS: m/z 415.3 (M+H)⁺.

### Example 24

### (R)—N-(2-(2-(3-(2-Benzylpiperazin-1-yl)-3-oxopropyl)phenoxy)phenethyl)acetamide

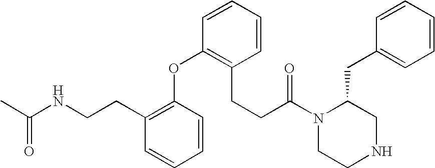

The title compound was prepared as described in Scheme 7 (Steps 1-4-a) via the following steps:

### Step A: (R)-tert-Butyl 3-benzyl-4-(3-(2-(2-formylphenoxy)phenyl)propanoyl)piperazine-1-carboxylate

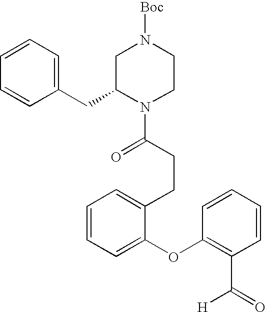

3-(2-(Benzoyloxy)phenyl)propanoic acid (2.07 g, 8.1 mmol) and (R)-tert-butyl 3-benzylpiperazine-1-carboxylate (2.09 g, 7.54 mmol) were dissolved in dichloromethane (30 mL) and EDCI (1.73 g, 9.00 mmol) was added followed by HOBt (1.44 g, 9.40 mmol). The reaction mixture was stirred at room temperature for 16 h, diluted with dichloromethane (200 mL), washed with water (200 mL), NaHCO₃ (sat. aq. 2×100 mL), dried (MgSO₄), filtered and concentrated in vacuo to afford 3.4 g of crude yellow oil. The residue was dissolved in tetrahydrofuran (200 mL) and dimethylformamide (10 mL). To the solution was added cesium carbonate (10.5 g, 32.2 mmol) and the reaction mixture was allowed to stir for 6 h at 80° C. The reaction mixture was cooled to room temperature, diluted with ethyl acetate (500 mL) and washed with water (2×200 mL) and brine (200 mL). The organic layer was separated, dried (Na₂SO₄) and concentrated in vacuo to afford 2.1 g of crude product as a thick oil. The oil was then dissolved in dimethylformamide (10 mL) and potassium carbonate was added (2.1 g, 15 mmol). To the stirred mixture was added 2-fluorobenzaldehyde (2.07 mL, 19.8 mmol) and the reaction was stirred for 12 h at 120° C. The dark brown solution was then cooled to room temperature and diluted with ethyl acetate (200 mL) and washed with water (3×200 mL) and brine (100 mL). The organic layer was separated, dried (Na₂SO₄), and concentrated in vacuo to a dark brown oil. Purification of the oil via column chromatography (20-35% ethyl acetate in hexanes) yielded 1.54 g (36%) of the title compound as a white solid. ESI-MS: m/z 529.4 (M+H)⁺.

### Step B: (R,E)-tert-butyl 3-benzyl-4-(3-(2-(2-(2-nitrovinyl)phenoxy)phenyl)propanoyl)-piperazine-1-carboxylate

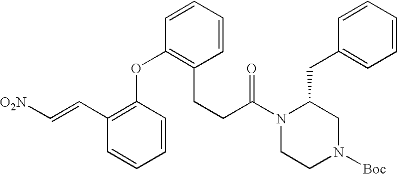

Ammonium acetate (60 mg, 0.73 mmol) was added to a flask containing (R)-tert-butyl 3-benzyl-4-(3-(2-(2-formylphenoxy)phenyl)propanoyl)piperazine-1-carboxylate (500 mg, 0.946 mmol) and nitromethane (3 mL) was added. The mixture was heated to 80° C. with stirring for 0.5 h and allowed to cool to room temperature. The reaction mixture was diluted with dichloromethane (10 mL) and washed with water (3×20 mL) and brine (1×20 mL). The organic layer was separated, dried (Na₂SO₄) and concentrated in vacuo to afford the title compound as a yellow amorphous solid (540 mg, 100%). ESI-MS: m/z 572.4 (M+H)⁺.

### Step C: (R)—N-(2-(2-(3-(2-Benzylpiperazin-1-yl)-3-oxopropyl)phenoxy)phenethyl)-acetamide

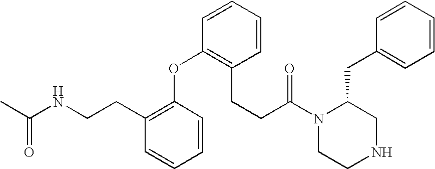

(R,E)-tert-butyl 3-benzyl-4-(3-(2-(2-(2-nitrovinyl)phenoxy)phenyl)propanoyl)piperazine-1-carboxylate (120 mg, 0.210 mmol) was dissolved in acetic anhydride (2 mL) and an aliquot (˜0.75 mL) of well-mixed Ni-Raney suspension in Ac₂O [prepared by washing ˜3 mL of commercial Ni-Raney suspension in water with water (3×5 mL), ethanol (3×5 mL), acetic anhydride (3×5 mL), in that order, and suspending it in acetic anhydride (5 mL)] was added to the yellow solution. The reaction mixture was charged with 50 psi hydrogen and heated to 50° C. with stirring for 1 h. The reaction was cooled to room temperature, diluted with dichloromethane (10 mL), and filtered through a plug of celite to remove catalyst. The clear solution was then concentrated to an oil and dissolved in dichloromethane (5 mL). Trifluoroacetic acid (2 mL) was added and the solution was allowed to stir at room temperature for 1 h. It was then concentrated to an oil in vacuo. The residue was purified by HPLC (30-60% acetonitrile in water, TFA buffered) to afford the title compound as a white semisolid (27.6 mg, 27%). ¹H NMR (400 MHz, DMSO-D6) δ, ppm: 1.72 (d, J=8.10 Hz, 6H), 2.31-3.30 (m, 26H), 3.91 (d, J=15.33 Hz, 1H), 4.35 (s, 1H), 4.51 (d, J=14.24 Hz, 1H), 4.83 (s, 1H), 6.61-6.70 (m, 4H), 6.97-7.30 (m, 22H), 7.92 (bs, 2H) 8.64 (bs, 2H), 8.98-9.31 (m, 2H). ESI-MS: m/z 485.8 (M+H)⁺.

### Example 25

### (R)—N-(2-(2-(3-(2-Benzylpiperazin-1-yl)-3-oxopropyl)-4-methylphenoxy)-1-methylphenethyl)acetamide

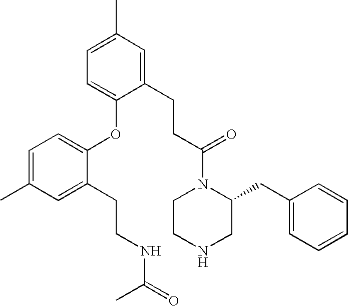

The title compound was prepared as described in Scheme 4 (Step 1) and Scheme 7 (Steps 1-4a) via the following steps:

### Step A: 6-Methylchroman-2-one

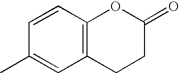

6-Methyl-2H-chromen-2-one (5.00 g, 31.2 mmol) was dissolved in tetrahydrofuran (30 mL) and Pd(OH)₂/C (20% w/w, 500 mg) was added. The reaction mixture was hydrogenated at 90 psi of hydrogen for 2 h at 50° C. It was then cooled, filtered through celite and concentrated in vacuo to yield 5.06 g (100%) of 6-methyl-2H-chromen-2-one as an oil. ESI-MS: m/z 163.3 (M+H)⁺.

### Step B: (R)-tert-Butyl 3-benzyl-4-(3-(2-hydroxy-5-methylphenyl)propanoyl)piperazine-1-carboxylate

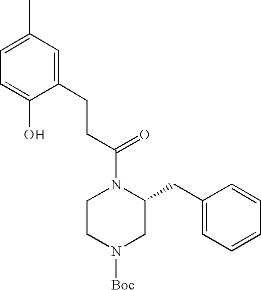

6-Methylchroman-2-one (1.10 g, 6.78 mmol) was dissolved in toluene (5 mL) and added to a solution of (R)-tert-butyl 3-benzylpiperazine-1-carboxylate (1.70 g, 6.15 mmol) in toluene (5 mL). The reaction mixture was stirred for 2 h at 110° C., cooled to room temperature and concentrated in vacuo to a viscous oil. Flash-column chromatography (30% ethyl acetate in hexanes) yielded the title compound as a white solid (1.90 g, 64%). ESI-MS: m/z 439.4 (M+H)⁺.

### Step C: (R)-tert-Butyl 3-benzyl-4-(3-(2-(2-formyl-4-methylphenoxy)-5-methylphenyl)propanoyl)piperazine-1-carboxylate

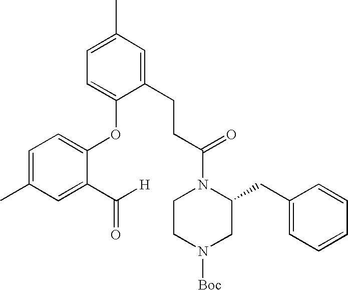

(R)-tert-Butyl 3-benzyl-4-(3-(2-hydroxy-5-methylphenyl)propanoyl)piperazine-1-carboxylate (150 mg, 0.342 mmol) was dissolved in dimethylformamide (0.5 mL) and potassium carbonate was added (95.0 mg, 0.687 mmol). To the stirred mixture was added 2-fluoro-5-methylbenzaldehyde (469 mg, 3.40 mmol) and the reaction was stirred for 12 h at 120° C. The dark brown solution was then cooled to room temperature, diluted with ethyl acetate (20 mL) and washed with water (3×20 mL) and brine (10 mL). The organic layer was dried (Na₂SO₄) and concentrated to a dark brown oil in vacuo. Purification of the oil via column chromatography (20-35% ethyl acetate in hexanes) yielded the title compound as a white solid (82 mg, 43%). ESI-MS: m/z 557.4 (M+H)⁺.

### Step D: (R)—N-(2-(2-(3-(2-Benzylpiperazin-1-yl)-3-oxopropyl)-4-methylphenoxy)-5-methylphenethyl)acetamide

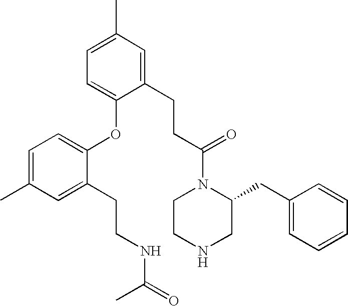

The title compound was prepared as described for Example 24, B-C. ¹H NMR (due to a ˜1:1 mixture of the tertiary amide rotamers, combined NMR spectral data for both rotamers are reported herein, leading to 2× hydrogen count) (400 MHz, DMSO-D6) δ, ppm: 1.72 (d, J=8.10 Hz, 7H), 2.23 (s, 12H), 2.26-3.22 (m, 31H), 3.91 (d, J=15.46 Hz, 1H), 4.31 (s, 1H), 4.51 (d, J=15.47 Hz, 1H), 4.82 (s, 1H), 6.46-6.56 (m, 4H), 6.80-7.07 (m, 8H), 7.16-7.24 (m, 10H), 7.92 (bs, 2H), 8.64 (bs, 2H), 8.98-9.31 (m, 2H). ESI-MS: m/z 514.5 (M+H)⁺.

### Example 26

### (R)—N-(2-(2-(3-(2-Benzylpiperazin-1-yl)-3-oxopropyl)-4-methylphenoxy)phenethyl)acetamide

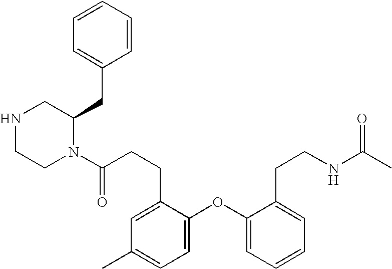

The title compound was prepared as described for Example 25. ESI-MS: m/z 500.4 (M+H)⁺.

### Example 27

### (R)—N-(2-(2-(3-(2-Benzylpiperazin-1-yl)-3-oxopropyl)-5-methylphenoxy)-1-methylphenethyl)acetamide

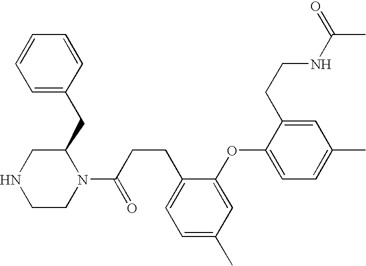

The title compound was prepared as described for Example 25. ESI-MS: m/z 514.5 (M+H)⁺.

### Example 28

### (R)—N-(2-(2-(3-(2-Benzylpiperazin-1-yl)-3-oxopropyl)-5-methylphenoxy)phenethyl)acetamide

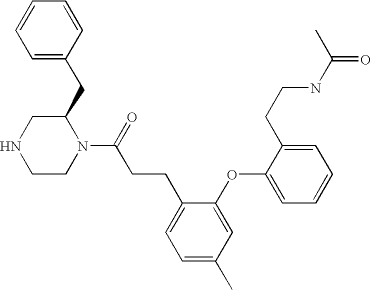

The title compound was prepared as described for Example 25. ESI-MS: m/z 500.4 (M+H)⁺.

### Example 29

### (R)—N-(2-(2-(3-(2-Benzylpiperazin-1-yl)-3-oxopropyl)phenoxy)benzyl)acetamide

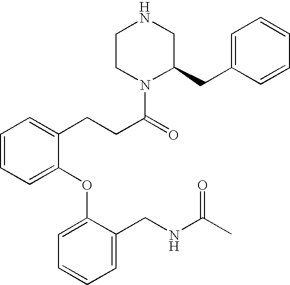

The title compound was prepared as described in Scheme 4 via the following steps:

### Step A: (R)-tert-Butyl 3-benzyl-4-(3-(2-(2-cyanophenoxy)phenyl)propanoyl)piperazine-1-carboxylate

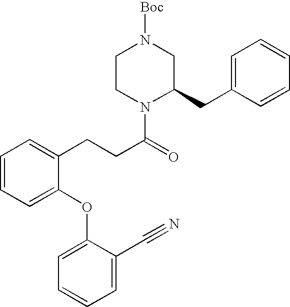

(R)-tert-Butyl 3-benzyl-4-(3-(2-hydroxyphenyl)propanoyl)piperazine-1-carboxylate (prepared as described in Example 25, B) (200 mg, 0.47 mmol) was dissolved in dimethylformamide (0.5 mL) and potassium carbonate (100 mg, 0.72 mmol) was added followed by 2-fluorobenzonitrile (568 mg, 4.7 mmol). The reaction mixture was stirred at 120° C. for 5 h. It was then cooled to room temperature and diluted with dichloromethane (10 mL). The organics were washed with water (10 mL), brine (5 mL), dried (Na₂SO₄) and concentrated in vacuo to afford the title compound as a yellow semisolid (215 mg, 87%), which was used in the next step without further purification. ESI-MS: m/z 526.4 (M+H)⁺.

### Step B: (R)-tert-butyl 4-(3-(2-(2-(acetamidomethyl)phenoxy)phenyl)propanoyl)-3-benzylpiperazine-1-carboxylate

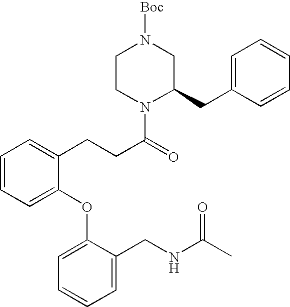

(R)-tert-Butyl 3-benzyl-4-(3-(2-(2-cyanophenoxy)phenyl)propanoyl)piperazine-1-carboxylate (215 mg, 0.409 mmol) was dissolved in acetic anhydride (4 mL) and an aliquot (˜1 mL) of well-mixed Ni-Raney suspension in Ac₂O [prepared by washing ˜3 mL of commercial Ni-Raney suspension in water with water (3×5 mL), ethanol (3×5 mL), acetic anhydride (3×5 mL), in that order, and suspending it in acetic anhydride (5 mL)] was added to the yellow solution. The reaction mixture was charged with 50 psi hydrogen and heated to 50° C. with stirring for 1 h. The reaction was cooled to room temperature, diluted with dichloromethane (10 mL), and filtered through a plug of celite to remove catalyst. The clear solution was then concentrated to an oil (230 mg, 98%), which was used in the next step without further purification. ESI-MS: m/z 572.5 (M+H)⁺.

### Step C: (R)—N-(2-(2-(3-(2-Benzylpiperazin-1-yl)-3-oxopropyl)phenoxy)benzyl)acetamide

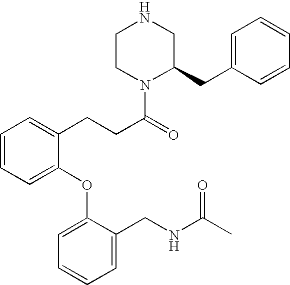

(R)-tert-Butyl 4-(3-(2-(2-(acetamidomethyl)phenoxy)phenyl)propanoyl)-3-benzylpiperazine-1-carboxylate (230 mg, 0.402 mmol) was dissolved in dichloromethane (5 mL). Trifluoroacetic acid (3 mL) was added and the solution was allowed to stir at room temperature for 1 h. It was then concentrated to an oil in vacuo. The residue was purified by HPLC (30-60% acetonitrile in water, TFA buffered) to afford the title compound as a white semisolid (10.9 mg, 4.9%). ESI-MS: m/z 472.4 (M+H)⁺.

### Example 30

### (R)-2-(2-(3-(2-benzylpiperazin-1-yl)-3-oxopropyl)phenoxy)benzonitrile

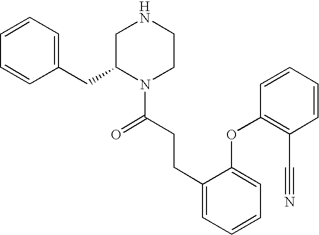

The title compound was prepared as described for Example 29, Step A, using (R)-1-(2,4-dibenzylpiperazin-1-yl)-3-(2-hydroxyphenyl)propan-1-one as a nucleophile and the resulting N-benzyl derivative was deprotected as described in Example 4, Step B. ESI-MS: m/z 426.3 (M+H)⁺.

### Example 31

### (R)-1-(2-Benzylpiperazin-1-yl)-3-(2-(3-(dimethylamino)phenoxy)phenyl)propan-1-one

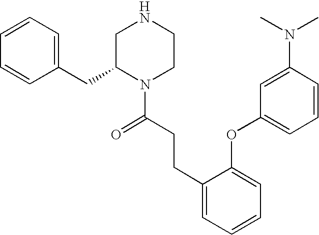

The title compound was prepared as described in Scheme 9. Specifically, (R)-tert-butyl 3-benzyl-4-(3-(2-hydroxyphenyl)propanoyl)piperazine-1-carboxylate (prepared in Example 25) (150 mg, 0.35 mmol) were dissolved in dichloromethane (2 mL) and 4 A molecular sieves were added (200 mg). To the mixture was added copper acetate (320 mg, 1.76 mmol) and pyridine (0.2 mL, 1.76 mmol). The solution was stirred at room temperature for 72 h and the reaction mixture was diluted with dichloromethane (50 mL), filtered through celite, then washed with water (25 mL) and brine (25 mL). The organic layer was dried and concentrated in vacuo to an oil. This was dissolved in dichloromethane (5 mL) and trifluoroacetic acid (3 mL) was added. After 1 h of stirring at room temperature the reaction mixture was concentrated and the residue was purified by HPLC (30-60% acetonitrile in water, TFA buffered) to afford the title compound as a white semisolid (35.3 mg, 23%). ESI-MS: m/z 444.4 (M+H)⁺.

### Example 32

### (R)-1-(2-Benzylpiperazin-1-yl)-3-(2-(3-ethoxyphenoxy)phenyl)propan-1-one

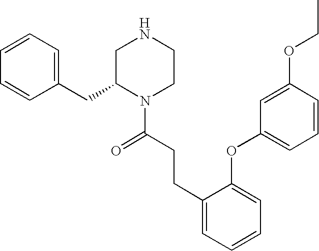

The title compound was prepared as described for Example 31. ESI-MS: m/z 445.4 (M+H)⁺.

### Example 33

### (R)—N-(3-(2-(3-(2-Benzylpiperazin-1-yl)-3-oxopropyl)phenoxy)phenyl)acetamide

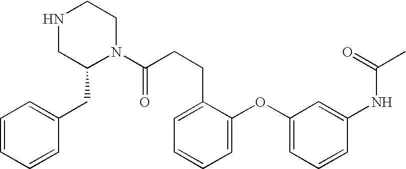

The title compound was prepared as described for Example 31. ESI-MS: m/z 458.4 (M+H)⁺.

### Example 34

### (R)-3-(2-(3-(2-Benzylpiperazin-1-yl)-3-oxopropyl)phenoxy)-N-ethylbenzamide

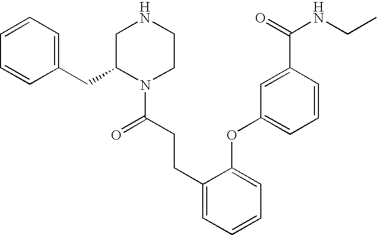

The title compound was prepared as described for Example 31. ESI-MS: m/z 472.4 (M+H)⁺.

### Example 35

### (R)-1-(2-Benzylpiperazin-1-yl)-3-(2-(3-(hydroxymethyl)phenoxy)phenyl)propan-1-one

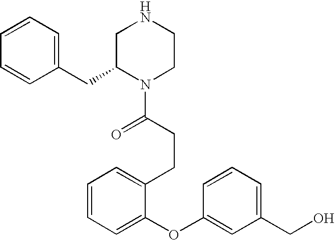

The title compound was prepared as described for Example 31. ESI-MS: m/z 431.4 (M+H)⁺.

### Example 36

### (R)-3-(2-(3-Acetylphenoxy)phenyl)-1-(2-benzylpiperazin-1-yl)propan-1-one

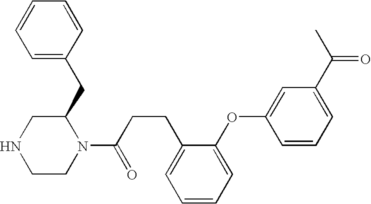

The title compound was prepared as described for Example 31. ESI-MS: m/z 443.4 (M+H)⁺.

### Example 37

### (R)-3-(2-(3-(2-benzylpiperazin-1-yl)-3-oxopropyl)phenoxy)benzamide

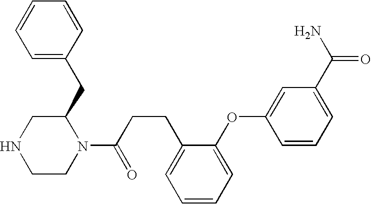

The title compound was prepared as described for Example 31. ESI-MS: m/z 444.4 (M+H)⁺.

### Example 38

### (R)—N-((2-(2-(3-(2-benzylpiperazin-1-yl)-3-oxopropyl)phenoxy)-pyridin-3-yl)methyl)acetamide

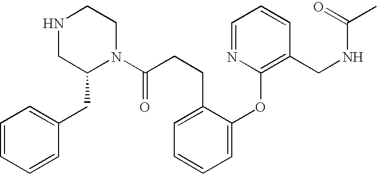

The title compound was prepared as described for Example 29. ESI-MS: m/z 473.4 (M+H)⁺.

### Example 39

### (R)—N-((2-(2-(3-(2-benzylpiperazin-1-yl)-3-oxopropyl)phenoxy)-4,6-dimethylpyridin-3-yl)methyl)acetamide

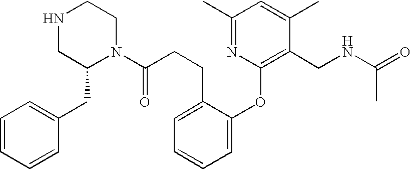

The title compound was prepared as described for Example 29. ESI-MS: m/z 501.4 (M+H)⁺.

### Example 40

### (R)—N-(2-(2-(3-(2-benzylpiperazin-1-yl)-3-oxopropyl)phenoxy)-5-methylbenzyl)acetamide

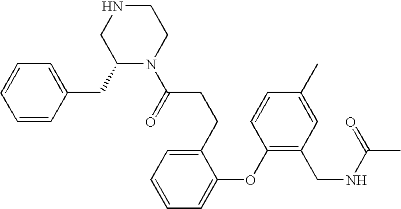

The title compound was prepared as described for Example 29. ESI-MS: m/z 486.4 (M+H)⁺.

### Example 41

### (R)—N-(2-(2-(3-(2-benzylpiperazin-1-yl)-3-oxopropyl)phenoxy)-5-methoxybenzyl)acetamide

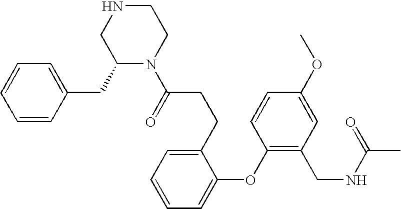

The title compound was prepared as described for Example 29. ESI-MS: m/z 502.4 (M+H)⁺.

### Example 42

### (R)—N-((2-(2-(3-(2-benzylpiperazin-1-yl)-3-oxopropyl)-4-methylphenoxy)-6-methylpyridin-3-yl)methyl)acetamide

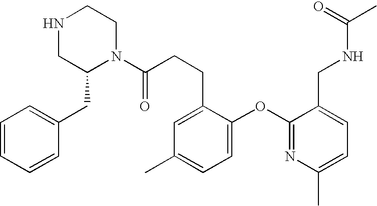

The title compound was prepared as described for Example 29. ESI-MS: m/z 501.4 (M+H)⁺.

### Example 43

### (R)—N-(2-(2-(3-(2-benzylpiperazin-1-yl)-3-oxopropyl)-4-methylphenoxy)-5-methylbenzyl)acetamide

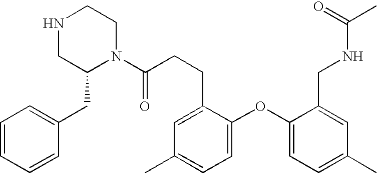

The title compound was prepared as described for Example 29. ESI-MS: m/z 500.4 (M+H)⁺.

### Example 44

### (R)—N-((2-(2-(3-(2-benzylpiperazin-1-yl)-3-oxopropyl)-5-methylphenoxy)-6-methylpyridin-3-yl)methyl)acetamide

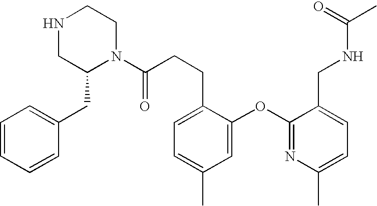

The title compound was prepared as described for Example 29. ESI-MS: m/z 501.4 (M+H)⁺.

### Example 45

### (R)—N-(2-(2-(3-(2-benzylpiperazin-1-yl)-3-oxopropyl)-5-methylphenoxy)-5-(trifluoromethyl)benzyl)acetamide

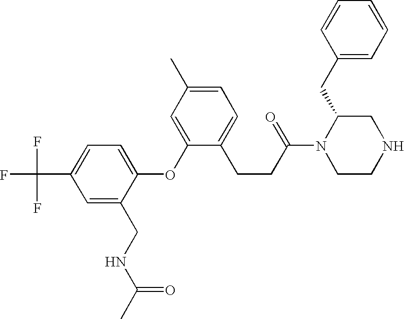

The title compound was prepared as described for Example 29. ESI-MS: m/z 554.5 (M+H)⁺.

### Example 46

### (R)—N-(2-(2-(3-(2-benzylpiperazin-1-yl)-3-oxopropyl)-5-methylphenoxy)-5-methylbenzyl)acetamide

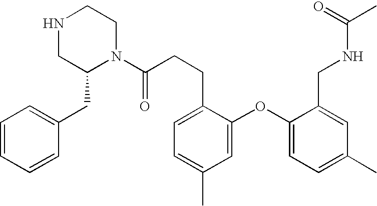

The title compound was prepared as described for Example 29. ESI-MS: m/z 500.4 (M+H)⁺.

### Example 47

### (R)—N-(2-(2-(3-(2-benzylpiperazin-1-yl)-3-oxopropyl)-4-methylphenoxy)-5-(trifluoromethyl)benzyl)acetamide

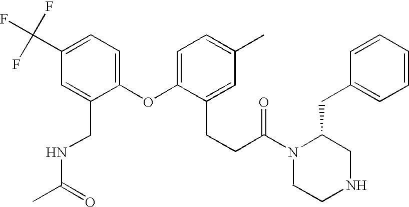

The title compound was prepared as described for Example 29. ESI-MS: m/z 554.4 (M+H)⁺.

### Example 48

### (R)—N-(3-(2-(2-(3-(2-benzylpiperazin-1-yl)-3-oxopropyl)phenoxy)-phenyl)propyl)acetamide

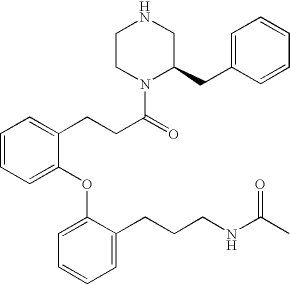

The title compound was prepared as described in Scheme 8 via the following steps:

### Step A: (R,E)-tert-butyl 3-benzyl-4-(3-(2-(2-(2-cyanovinyl)phenoxy)phenyl)propanoyl)piperazine-1-carboxylate

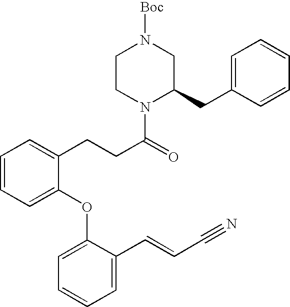

(R)-tert-butyl 3-benzyl-4-(3-(2-(2-formylphenoxy)phenyl)propanoyl)piperazine-1-carboxylate (100 mg, 0.189 mmol) was dissolved in toluene (0.3 mL) and (triphenylphosphoranylidene) acetonitrile (75 mg, 0.25 mmol) was added. The reaction mixture was heated to 90° C. and stirred for 2 h. The crude reaction mixture was then concentrated to a thick oil and purified via flash chromatography (30% ethyl acetate in hexanes) to yield 95 mg (91%) of the title compound as a semi-solid. ESI-MS: m/z 552.4 (M+H)⁺.

### Step B: (R)—N-(3-(2-(2-(3-(2-benzylpiperazin-1-yl)-3-oxopropyl)phenoxy)phenyl)propyl)acetamide

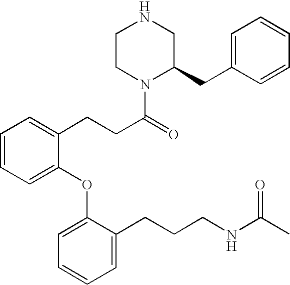

(R,E)-tert-butyl 3-benzyl-4-(3-(2-(2-(2-cyanovinyl)phenoxy)phenyl)propanoyl)piperazine-1-carboxylate (50 mg, 0.09 mmol) was dissolved in acetic anhydride (1 mL) and an aliquot (˜0.5 mL) of well-mixed Ni-Raney suspension in Ac₂O [prepared by washing ˜3 mL of commercial Ni-Raney suspension in water with water (3×5 mL), ethanol (3×5 mL), acetic anhydride (3×5 mL), in that order, and suspending it in acetic anhydride (5 mL)] was added to the yellow solution. The reaction mixture was charged with 50 psi hydrogen and heated to 50° C. with stirring for 1 h. The reaction was cooled to room temperature, diluted with dichloromethane (10 mL), and filtered through a plug of celite to remove catalyst. The clear solution was then concentrated to an oil and dissolved in dichloromethane (5 mL). Trifluoroacetic acid (3 mL) was added and the solution was allowed to stir at room temperature for 1 h. It was then concentrated to an oil in vacuo. The residue was purified by HPLC (30-60% acetonitrile in water, TFA buffered) to afford the title compound as a white semisolid (5.0 mg, 11.1%). ESI-MS: m/z 500.5 (M+H)⁺.

### Example 49

### (R)—N-(2-(2-(3-(2-benzylpiperazin-1-yl)-3-oxopropyl)phenoxy)-5-methylphenethyl)acetamide

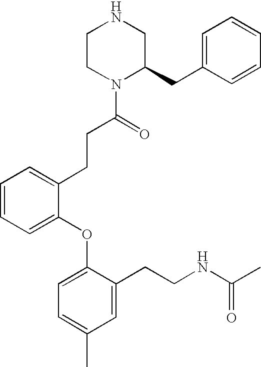

The title compound was prepared as described for Example 25. ESI-MS: m/z 500.5 (M+H)⁺.

### Example 50

### (R)-1-(2-benzylpiperazin-1-yl)-3-(2-(p-tolyloxy)phenyl)propan-1-one

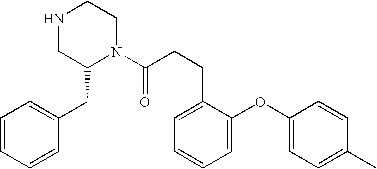

The title compound was prepared as described for Example 21. The crude product was purified by HPLC to afford the title compound as a white solid (TFA salt; 95.0 mg, 31%). ESI-MS: m/z 415.4 (M+H)⁺.

### Example 51

### (R)-1-(2-benzylpiperazin-1-yl)-3-(2-(4-methoxyphenoxy)-phenyl)propan-1-one

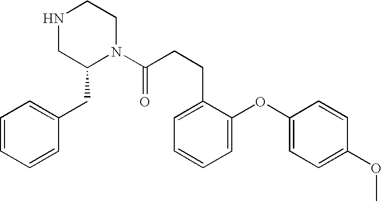

The title compound was prepared as described for Example 50. ESI-MS: m/z 431.4 (M+H)⁺.

### Example 52

### 2-(3-((R)-2-benzylpiperazin-1-yl)-3-oxopropyl)-2-phenylcyclohexanone

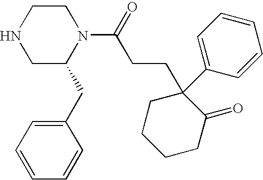

The title compound was prepared as described for Example 50. ESI-MS: m/z 405 (M+H)⁺.

### Example 53

### (R)-3-(2-benzylphenyl)-1-(2-benzylpiperazin-1-yl)propan-1-one

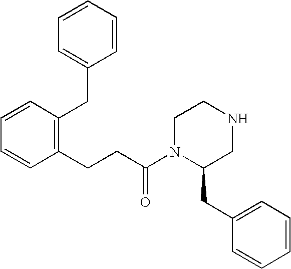

The title compound was prepared as described for Example 50. ESI-MS: m/z 399 (M+H)⁺.

### Example 54

### (R)-1-(2-benzylpiperazin-1-yl)-3-(4-(4-chlorophenyl)-2-methyloxazol-5-yl)propan-1-one

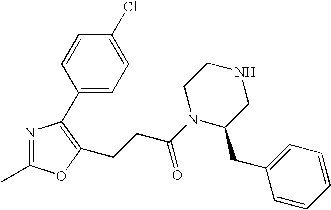

The title compound was prepared as described for Example 50. ESI-MS: m/z 424 (M+H)⁺.

### Example 55

### (R)-3-(3-(2-benzylpiperazin-1-yl)-3-oxopropyl)-2H-benzo[b][1,4]-oxazin-2-one

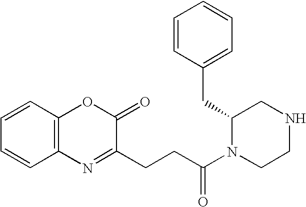

The title compound was prepared as described for Example 50. ESI-MS: m/z 378 (M+H)⁺.

### Example 56

### (R)-1-(2-benzylpiperazin-1-yl)-3-(2-(4-chlorobenzyl)phenyl)propan-1-one

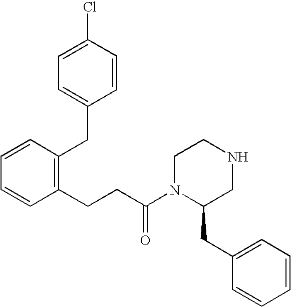

The title compound was prepared as described for Example 50. ESI-MS: m/z 433 (M+H)⁺.

### Example 57

### (R)-1-(2-benzylpiperazin-1-yl)-2-(2-methoxyphenoxy)ethanone

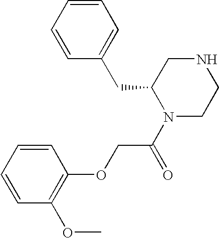

The title compound was prepared as described for Example 50. ESI-MS: m/z 341 (M+H)⁺.

### Example 58

### (R)-1-(2-benzylpiperazin-1-yl)-3-(diphenylamino)propan-1-one

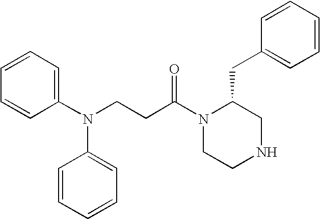

The title compound was prepared as described for Example 50. ESI-MS: m/z 400 (M+H)⁺.

### Example 59

### (R)-4-(3-(2-benzylpiperazin-1-yl)-3-oxopropyl)-2-methoxyphenyl acetate

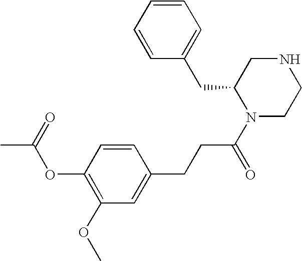

The title compound was prepared as described for Example 50. ESI-MS: m/z 397 (M+H)⁺.

### Example 60

### (R)-4-(2-benzylpiperazin-1-yl)-4-oxo-N,N-diphenylbutanamide

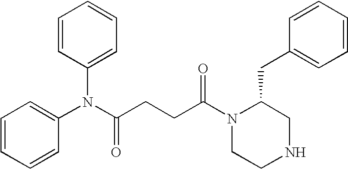

The title compound was prepared as described for Example 50. ESI-MS: m/z 428 (M+H)⁺.

### Example 61

### 1-((R)-2-benzylpiperazin-1-yl)-3-(2,2-dimethyltetrahydro-2H-pyran-4-yl)-3-phenylpropan-1-one

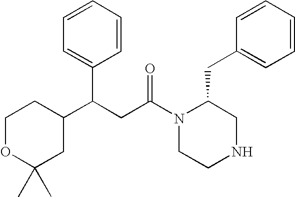

The title compound was prepared as described for Example 50. ESI-MS: m/z 421 (M+H)⁺.

### Example 62

### (R)-2-(3-benzylphenoxy)-1-(2-benzylpiperazin-1-yl)ethanone

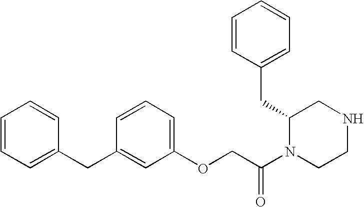

The title compound was prepared as described for Example 50. ESI-MS: m/z 401 (M+H)⁺.

### Example 63

### (R)-1-(2-benzylpiperazin-1-yl)-3-(4,5-diphenyloxazol-2-yl)propan-1-one

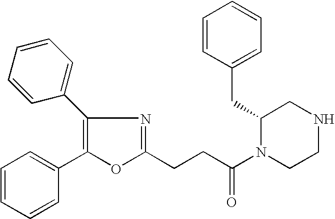

The title compound was prepared as described for Example 50. ESI-MS: m/z 452 (M+H)⁺.

### Example 64

### (R)—N-(1-(2-(2-benzylpiperazin-1-yl)-2-oxoethyl)cyclohexyl)benzamide

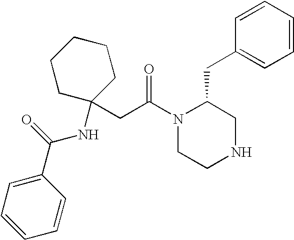

The title compound was prepared as described for Example 50. ESI-MS: m/z 420 (M+H)⁺.

### Example 65

### (R)-2-(benzhydrylthio)-1-(2-benzylpiperazin-1-yl)ethanone

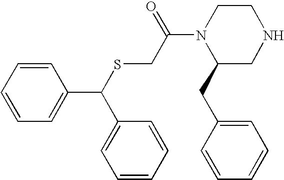

The title compound was prepared as described for Example 50. ESI-MS: m/z 417 (M+H)⁺.

### Example 66

### (R)-3-(2-(2-(3-(2-benzylpiperazin-1-yl)-3-oxopropyl)phenoxy)phenyl)propanenitrile

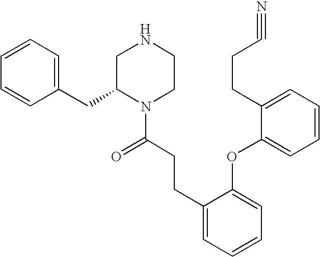

(R,E)-tert-butyl 3-benzyl-4-(3-(2-(2-(2-cyanovinyl)phenoxy)phenyl)propanoyl)piperazine-1-carboxylate (prepared in Example 48, 50 mg, 0.09 mmol) was dissolved in THF (5 mL) and Pd/C (10%, 5 mg) was added. The reaction mixture was hydrogenated under 1 atm of hydrogen at room temperature for 2 h and was then filtered through a plug of celite. The reaction mixture was concentrated in vacuo, dissolved in dichloromethane (5 mL) and trifluoroacetic acid (3 mL) was added. The mixture was allowed to stir at room temperature for 1 h and then concentrated to an oil. The residue was purified by HPLC (30-60% acetonitrile in water, TFA buffered) to afford the title compound as a white semisolid (7.0 mg, 17.1%). ESI-MS: m/z 454.4 (M+H)⁺.

### Example 67

### (R)-methyl 3-(2-(2-(3-(2-benzylpiperazin-1-yl)-3-oxopropyl)phenoxy)phenyl)propanoate

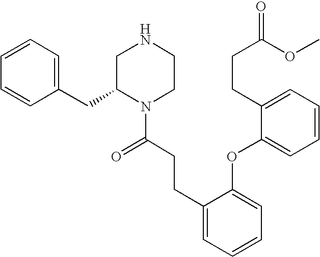

The title compound was prepared as outlined in Scheme 7, steps 1-4-b) using procedures in Example 66 with (triphenylphosphoranylidene) acetate as an ylide reagent. ESI-MS: m/z 487.4 (M+H)⁺.

### Example 68

### (R)-1-(2-(4-hydroxybenzyl)piperazin-1-yl)-3-(2-phenoxyphenyl)-propan-1-one

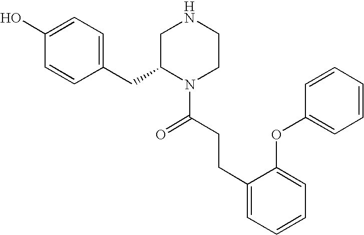

The title compound was prepared as described for Example 3, using (R)-4-((4-benzylpiperazin-2-yl)methyl)phenol (prepared as described in Scheme 1) as an amine component (Scheme 2). ESI-MS: m/z 417.4 (M+H)⁺.

### Example 69

### (R)-methyl 2-(4-((1-(3-(2-phenoxyphenyl)propanoyl)piperazin-2-yl)methyl)phenoxy)acetate

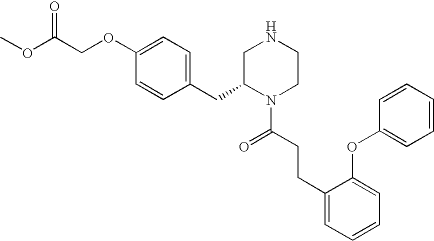

The title compound was prepared as described in Scheme 6. Specifically, (R)-tert-Butyl 3-(4-hydroxybenzyl)-4-(3-(2-phenoxyphenyl)propanoyl)piperazine-1-carboxylate (70 mg, 0.13 mmol) was dissolved in acetone (0.5 mL) and potassium carbonate (38 mg, 0.28 mmol) was added followed by methyl 2-chloroacetate (17 mg, 0.16 mmol). The reaction was allowed to stir at 80° C. for 16 h. The reaction was then cooled to room temperature, filtered through celite, and concentrated to an oil. The crude oil was then dissolved in dioxane (5 mL) and ammonium formate (32 mg, 0.52 mmol) followed by the addition of Pd(OH)₂ (25 mg, 10% by weight on carbon). The reaction mixture was stirred at 80° C. for 1 h in a sealed vial and then cooled to room temperature. The mixture was filtered through celite and concentrated to an oil. The residue was purified by HPLC (30-60% acetonitrile in water, TFA buffered) to afford the title compound as a white semisolid (7.0 mg, 11.0%). ¹H NMR (due to a ˜1:1 mixture of the tertiary amide rotamers, combined NMR spectral data for both rotamers are reported herein, leading to 2× hydrogen count) (400 MHz, DMSO-D6) δ, ppm: 1.80-1.90 (m, 2H) 2.41-3.38 (m, 20H), 3.66 (s, 6H), 3.86 (d, J=12.91 Hz, 1H), 4.25 (s, 1H), 4.46 (d, J=15.50 Hz, 1H), 4.72 (s, 4H), 4.74 (s, 1H), 6.78-6.91 (m, 5H), 7.06-7.34 (m, 8H), 8.74 (bs, 2H), 9.31 (bs, 2H). ESI-MS: ESI-MS: m/z 489.4 (M+H)⁺.

### Example 70

### (R)-2-(4-((1-(3-(2-phenoxyphenyl)propanoyl)piperazin-2-yl)methyl)phenoxy)acetamide

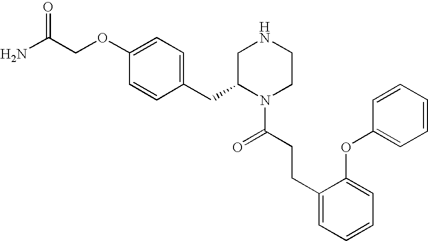

The title compound was prepared as described for Example 69 using 2-chloroacetamide as an electrophile. ESI-MS: m/z 474.4 (M+H)⁺.

### Example 71

### (R)—N,N-dimethyl-2-(4-((1-(3-(2-phenoxyphenyl)propanoyl)piperazin-2-yl)methyl)phenoxy)acetamide

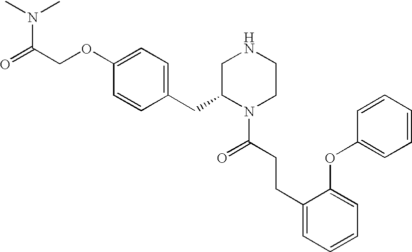

The title compound was prepared as described for Example 69 using 2-chloro-N,N-dimethylacetamide as an electrophile. ESI-MS: m/z 502.4 (M+H)⁺.

### Example 72

### (R)-4-((1-(3-(2-phenoxyphenyl)propanoyl)piperazin-2-yl)methyl)-phenyl 4-methylpiperazine-1-carboxylate

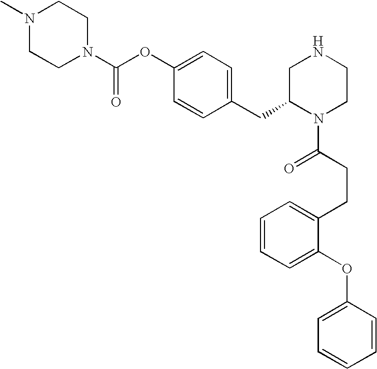

The title compound was prepared as described in Scheme 6 via the following steps:

### Step A: (R)-1-(4-Benzyl-2-(4-hydroxybenzyl)piperazin-1-yl)-3-(2-phenoxyphenyl)-propan-1-one

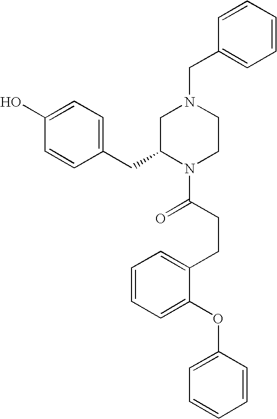

(R)-4-((4-benzylpiperazin-2-yl)methyl)phenol (prepared as described in Scheme 1, 0.664 g, 2.35 mmol), 3-(2-phenoxyphenyl)propanoic acid (0.600 g, 2.48 mmol) and EDCI (0.600 g, 3.13 mmol) were dissolved in DMF (2 mL). HOBt (0.480 g, 3.14 mmol) was added and the reaction mixture was stirred for 18 h. It was diluted with water (20 mL) and extracted with ethyl acetate (2×10 mL). The combined organic extracts were washed with water (2×5 mL), NaHCO₃ (sat. aq., 2×10 mL), brine (10 mL), dried (MgSO₄), filtered and concentrated in vacuo The crude material was purified using flash column chromatography on silica gel (90 g SiO₂, hexanes-ethyl acetate-dichloromethane 40:40:5) to afford the title compounds as an off-white solid. ESI-MS: m/z 507.4 (M+H)⁺.

### Step B: (R)-4-((1-(3-(2-phenoxyphenyl)propanoyl)piperazin-2-yl)methyl)phenyl 4-methylpiperazine-1-carboxylate

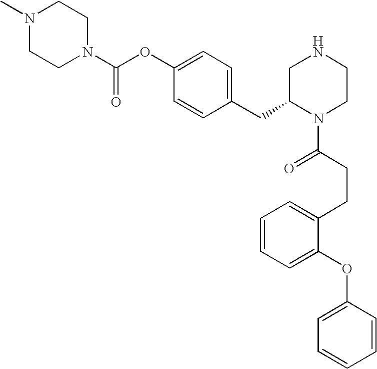

(R)-1-(4-benzyl-2-(4-hydroxybenzyl)piperazin-1-yl)-3-(2-phenoxyphenyl)propan-1-one (61.0 mg, 0.120 mmol) was dissolved in pyridine (0.2 mL) and morpholine-4-carbonyl chloride (40 mg, 0.267 mmol) was added. The reaction mixture was stirred at 100° C. for 1 h, and concentrated in vacuo. This crude product was dissolved in dioxane (2 mL) and ammonium formate (0.400 g, 6.34 mmol) was added followed by Pd(OH)₂/C (20%, 0.400 g). The mixture was sealed and heated at 80° C. for 1 h. The reaction mixture was cooled, filtered through celite using ethyl acetate (10 mL) to rinse the celite. The filtrate was concentrated in vacuo and the crude product was purified by HPLC (25-40% acetonitrile in water, TFA buffered) to afford the title compounds as a white solid (TFA salt, 39 mg, 50%). ESI-MS: m/z 543.5 (M+H)⁺.

### Example 73

### (R)-4-((1-(3-(2-phenoxyphenyl)propanoyl)piperazin-2-yl)methyl)-phenyl morpholine-4-carboxylate

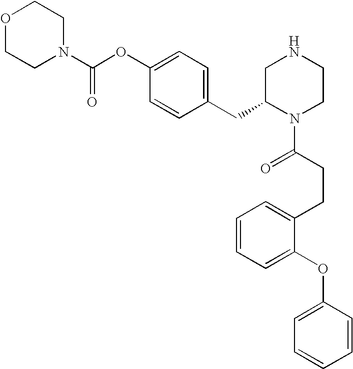

The title compound was prepared as described for Example 72. ESI-MS: m/z 530.4 (M+H)⁺.

### Example 74

### (R)-4-((1-(3-(2-phenoxyphenyl)propanoyl)piperazin-2-yl)methyl)-phenyl diethylcarbamate

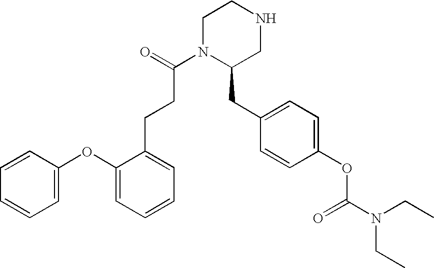

The title compound was prepared as described for Example 72. ESI-MS: m/z 516.5 (M+H)⁺.

### Example 75

### (R)-1-(2-benzylpiperazin-1-yl)-3-(2-(3-methoxypropoxy)phenyl)-propan-1-one

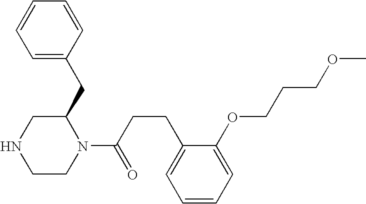

The title compound was prepared as described in Scheme 5. Specifically, (R)-tert-butyl 3-benzyl-4-(3-(2-hydroxyphenyl)propanoyl)piperazine-1-carboxylate (150 mg, 0.353 mmol) was dissolved in DMF (1 mL), and K₂CO₃ (400 mg, 2.89 mmol) was added, followed by 1-bromo-3-methoxypropane (0.120 g, 0.790 mmol). The reaction mixture was stirred at 90° C. for 3 h, diluted with water (3 mL) and extracted with ethyl acetate (3×2 mL). The combined organic extracts were dried (MgSO₄), filtered and concentrated in vacuo. The residue was dissolved in dichloromethane (2 mL), treated with TFA (1 mL), stirred for 15 min and concentrated in vacuo. The crude product was purified by HPLC (acetonitrile-water buffered with 0.05% TFA) to afford the title compound as a clear viscous oil (109 mg, 60%). ESI-MS: m/z 397.4 (M+H)⁺.

### Example 76

### (R)-1-(2-benzylpiperazin-1-yl)-3-(2-(3-hydroxypropoxy)phenyl)-propan-1-one

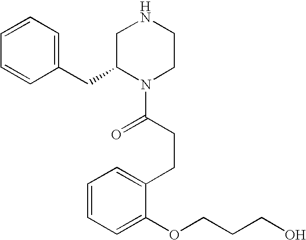

The title compound was prepared as described for Example 75. ESI-MS: m/z 383.4 (M+H)⁺.

### Example 77

### (R)-1-(2-benzylpiperazin-1-yl)-3-(2-(3-(2-methoxyethoxy)propoxy)phenyl)propan-1-one

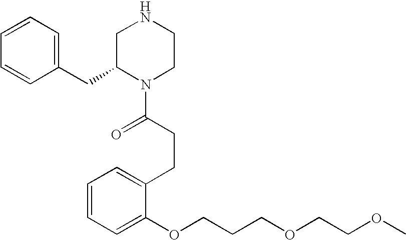

The title compound was prepared as described for Example 75. ESI-MS: m/z 441.4 (M+H)⁺.

### Example 78

### (R)—N-(2-(2-(3-(2-benzylpiperazin-1-yl)-3-oxopropyl)phenoxy)phenethyl)propionamide

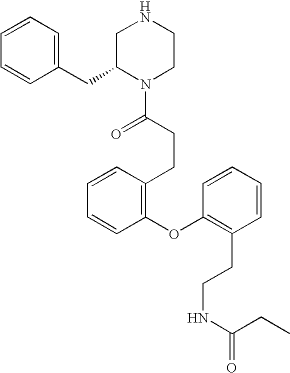

The title compound was prepared as described for Example 24, using propanoic anhydride in place of acetic anhydride. ESI-MS: m/z 500.5 (M+H)⁺.

### Example 79

### (R)—N-(2-(2-(3-(2-(4-hydroxybenzyl)piperazin-1-yl)-3-oxopropyl)phenoxy)phenethyl)acetamide

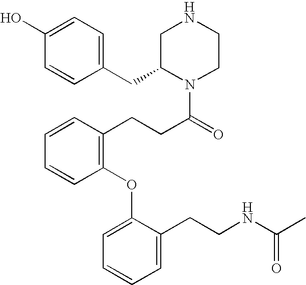

The title compound was prepared as described in Scheme 11 via the following steps:

### Step A: N-(2-(2-formylphenoxy)phenethyl)acetamide

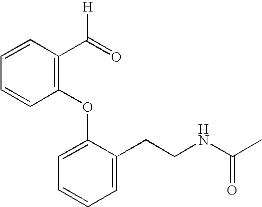

N-(2-hydroxyphenethyl)acetamide (500 mg, 2.78 mmol) was dissolved in dimethylformamide (1 mL) and potassium carbonate was added (770 mg, 5.6 mmol). The reaction mixture was heated to 120° C. for 8 h. The reaction mixture was then cooled to room temperature and dissolved in dichloromethane (100 mL). The mixture was washed with water (2×50 mL) and brine (25 mL). The organic layer was separated, dried (Na₂SO₄), and concentrated to a brown oil. The crude product was then purified via column chromatography (30% ethyl acetate in hexanes) to yield the title compound as a clear oil (394 mg, 50%). ESI-MS: m/z 284.4 (M+H)⁺.

### Step B: (E)-methyl 3-(2-(2-(2-acetamidoethyl)phenoxy)phenyl)acrylate

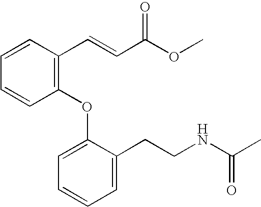

N-(2-(2-formylphenoxy)phenethyl)acetamide (369 mg, 1.30 mmol) was dissolved in toluene (2 mL) and methyl(triphenylphosphoranylidene)acetate (566 mg, 1.69 mmol) was added. The reaction mixture was stirred at 80° C. for 3 h, cooled to room temperature, and dissolved in ethyl acetate (50 mL). It was washed with water (2×30 mL) and brine (25 mL), dried (Na₂SO₄), and concentrated to an oil. The crude oil was taken to the next step without further purification. ESI-MS: m/z 340.4 (M+H)⁺.

### Step C: 3-(2-(2-(2-acetamidoethyl)phenoxy)phenyl)propanoic acid

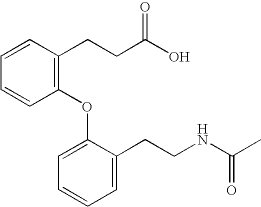

(E)-methyl 3-(2-(2-(2-acetamidoethyl)phenoxy)phenyl)acrylate (400 mg, 1.17 mmol) was dissolved in ethanol (5 mL), Pd/C (10% w/w, 40 mg) was added, and the reaction mixture was hydrogenated at 1 atm H₂ overnight at room temperature and for 2 h at 50° C. The reaction mixture was then cooled to room temperature and filtered through a plug of celite. To the crude reaction mixture was added NaOH (1N aq., 2 mL) and the reaction mixture was stirred at room temperature for 3 h. The reaction was then acidified to pH4 with HCl (1N aq., 2 mL). The excess ethanol was removed in vacuo and the product was extracted using diethyl ether (4×25 mL). The organics were concentrated to an oil and purified via column chromatography (5:15:80 methanol:ethyl acetate:dichloromethane) to yield the title compounds as an amorphous solid (234 mg, 42%). ESI-MS: m/z 328.4 (M+H)⁺.

### Step D: (R)—N-(2-(2-(3-(2-(4-hydroxybenzyl)piperazin-1-yl)-3-oxopropyl)phenoxy)-phenethyl)acetamide

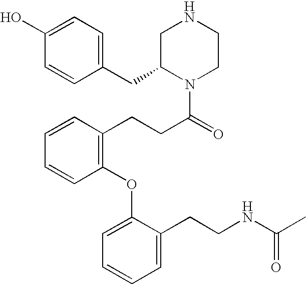

The title compound was prepared as described for Example 3 using DMF as a solvent instead of dichloromethane. ¹H NMR (due to a ˜1:1 mixture of the tertiary amide rotamers, combined NMR spectral data for both rotamers are reported herein, leading to 2× hydrogen count) (400 MHz, DMSO-D6) δ, ppm: 1.70 (bs, 6H), 2.61-3.31 (m, 30H), 3.90 (d, J=12.91 Hz, 1H), 4.25 (s, 1H), 4.46 (d, J=15.50 Hz, 1H), 4.70 (s, 1H), 6.60-6.74 (m, 8H), 6.92-7.07 (m, 8H), 7.11-7.22 (m, 4H), 7.23-7.33 (m, 4H), 7.93 (bs, 2H), 8.74 (bs, 2H), 9.02 (bs, 2H), 9.31 (bs, 2H). ESI-MS: m/z 502.5 (M+H)⁺.

### Example 80

### (R)-4-((1-(3-(2-(2-(2-acetamidoethyl)phenoxy)phenyl)propanoyl)piperazin-2-yl)methyl)phenyl morpholine-4-carboxylate

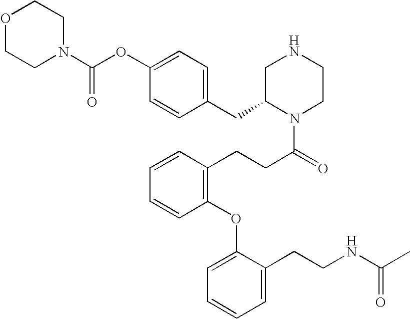

The title compound was prepared as described for Example 72, starting with (R)—N-(2-(2-(3-(4-benzyl-2-(4-hydroxybenzyl)piperazin-1-yl)-3-oxopropyl)phenoxy)phenethyl)acetamide and using morpholine-4-carbonyl chloride as an electrophile. ESI-MS: m/z 615.5 (M+H)⁺.

### Example 81

### (R)-2-(4-((1-(3-(2-(2-(2-acetamidoethyl)phenoxy)phenyl)propanoyl)piperazin-2-yl)methyl)phenoxy)acetamide

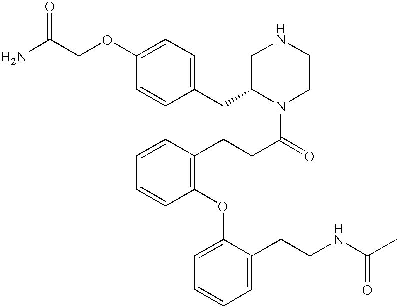

The title compound was prepared as described for Example 69. ESI-MS: m/z 559.5 (M+H)⁺.

### Example 82

### (R)-3-(2-(2-(3-(2-Benzylpiperazin-1-yl)-3-oxopropyl)phenoxy)phenyl)propanoic acid

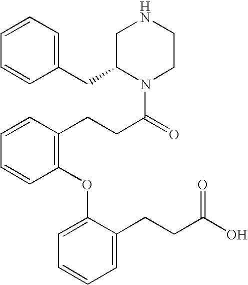

### Step A: (R,E)-tert-Butyl 3-benzyl-4-(3-(2-(2-(3-methoxy-3-oxoprop-1-enyl)phenoxy)phenyl)propanoyl)piperazine-1-carboxylate

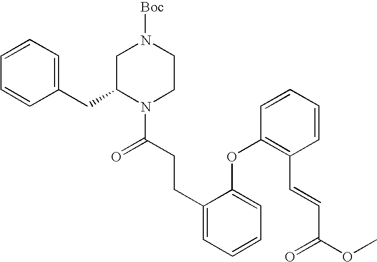

(R)-tert-Butyl 3-benzyl-4-(3-(2-(2-formylphenoxy)phenyl)propanoyl)piperazine-1-carboxylate (300 mg, 0.567 mmol) and (triphenylphosphoranylidene)acetate (247 mg, 0.738 mmol) were heated in toluene (2 mL) at 80° C. for 2 h. The reaction mixture was cooled to room temperature and diluted with ethyl acetate (50 mL). It was washed with water (2×30 mL) and brine (25 mL), dried (Na₂SO₄), and concentrated in vacuo. The residue was purified by flash column chromatography on silica gel (25 g SiO₂, 30% ethyl acetate in hexanes) to afford the title compound as a clear oil (301 mg, 92%). ESI-MS: m/z 585.5 (M+H)⁺.

### Step B: (R)-tert-Butyl 3-benzyl-4-(3-(2-(2-(3-methoxy-3-oxopropyl)phenoxy)phenyl)propanoyl)piperazine-1-carboxylate

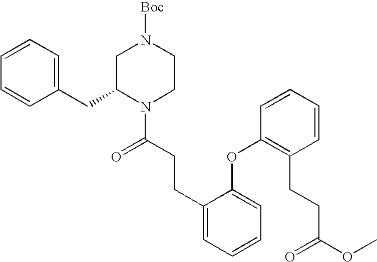

(R,E)-tert-Butyl 3-benzyl-4-(3-(2-(2-(3-methoxy-3-oxoprop-1-enyl)phenoxy)phenyl)-propanoyl)piperazine-1-carboxylate (301 mg, 0.515 mmol) was dissolved in THF (10 mL) and Pd/C (10% w/w, 30 mg) was added. The reaction mixture was hydrogenated at 1 atm H₂ at room temperature for 2 h, filtered through celite and concentrated in vacuo to afford the title compound as a clear oil (270 mg, 91%), which was used in the next step without further purification. ESI-MS: m/z 587.5 (M+H)⁺.

### Step C: (R)-3-(2-(2-(3-(2-Benzyl-4-(tert-butoxycarbonyl)piperazin-1-yl)-3-oxopropyl)phenoxy)phenyl)propanoic acid

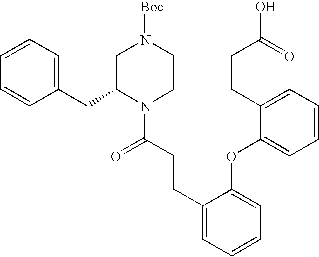

(R)-tert-Butyl 3-benzyl-4-(3-(2-(2-(3-methoxy-3-oxopropyl)phenoxy)phenyl) propanoyl)-piperazine-1-carboxylate (270 mg, 0.471 mmol) was dissolved in EtOH (4 mL) and treated with NaOH (1N, 2 mL). The reaction mixture was stirred at room temperature for 2 h and then acidified to pH4 with HCl (1N aq., 2 mL). The excess ethanol was removed in vacuo and the product was extracted using ethyl acetate (4×25 mL). The combined organic extracts were dried (Na₂SO₄), filtered and concentrated in vacuo to afford the title compound as a semi-solid (274 mg, 91%). ESI-MS: m/z 573.5 (M+H)⁺.

### Step D: (R)-3-(2-(2-(3-(2-Benzylpiperazin-1-yl)-3-oxopropyl)phenoxy)phenyl)propanoic acid

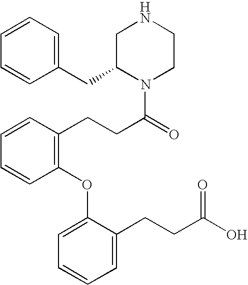

(R)-3-(2-(2-(3-(2-Benzyl-4-(tert-butoxycarbonyl)piperazin-1-yl)-3-oxopropyl)phenoxy)-phenyl)propanoic acid (100 mg, 0.170 mmol) was dissolved in dichloromethane (3 mL) and treated with TFA (2 mL). The reaction mixture was stirred for 2 h at room temperature and concentrated in vacuo. The residue was purified by HPLC (30-60% acetonitrile in water, TFA buffered) to afford the title compound as a white semisolid (32.0 mg, 40%). ESI-MS: m/z 473.5 (M+H)⁺.

### Example 83

### (R)-3-(2-(2-(3-(2-benzylpiperazin-1-yl)-3-oxopropyl)phenoxy)phenyl)propanamide

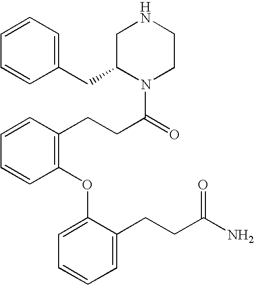

The title compound was prepared as described in Scheme 7 (Steps 1-6d). Specifically, (R)-3-(2-(2-(3-(2-benzyl-4-(tert-butoxycarbonyl)piperazin-1-yl)-3-oxopropyl)phenoxy)-phenyl)propanoic acid (165 mg, 0.288 mmol) was dissolved in DMF (3 mL) and NH₄Cl (17.1 mg, 0.320 mmol), TEA (61.7 mg, 0.610 mmol), EDCI (67.1 mg, 0.350 mmol) and HOBt (55.1 mg, 0.360 mmol) were added. The reaction mixture was stirred for 2 hours at room temperature, diluted with dichloromethane (10 mL), washed with water (5 mL), NaHCO₃ (sat. aq. 2×10 mL), dried (Na₂SO₄), filtered and concentrated in vacuo to an oil. It was dissolved in dichloromethane (3 mL), treated with TFA (2 mL), stirred at room temperature for 2 h and concentrated in vacuo. The residue was purified by HPLC (acetonitrile-water, TFA buffered) to afford the title compound as a white solid (40.0 mg, 29%). ESI-MS: m/z 472.4 (M+H)⁺.

### Example 84

### (R)—N-(2-(2-(3-(2-benzylpiperazin-1-yl)-3-oxopropyl)benzyl)phenethyl)acetamide

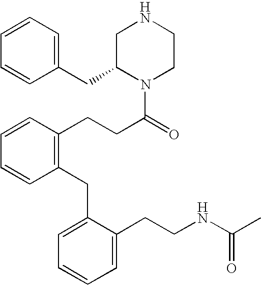

The title compound was prepared as described in Scheme 12 (Steps 1-7b) via the following steps:

### Step A: 2-(2-(Hydroxymethyl)phenyl)acetonitrile

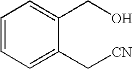

Methyl 2-(cyanomethyl)benzoate (6.00 g, 18.6 mmol) was dissolved in tetrahydrofuran (200 mL) and CaCl₂ (6.00 g, 54.1 mmol) was added. The reaction mixture was heated to 50° C. To the solution was added NaBH₄ (12.0 g, 317 mmol) portion-wise over the course of 12 h and the reaction was allowed to stir for 16 h at 50° C. The reaction mixture was diluted with ethyl acetate (200 mL) and poured slowly into a saturated NaHCO₃ aqueous solution (500 mL). The reaction was stirred at room temperature for 0.5 h, the organic layer was separated, dried (Na₂SO₄), and concentrated in vacuo. The residue was purified via column chromatography (20% ethyl acetate in dichloromethane) to yield 2.12 g (77%) of the product as a clear oil. ¹H NMR (400 MHz, DMSO-d₆) δ ppm 4.04 (s, 2H) 4.57 (d, J=5.31 Hz, 2H) 5.29 (t, J=5.43 Hz, 1H) 7.29-7.40 (m, 3H) 7.44 (d, J=6.82 Hz, 1H).

### Step B: 2-(Cyanomethyl)benzyl methyl carbonate

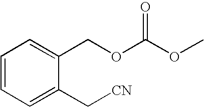

2-(2-(Hydroxymethyl)phenyl)acetonitrile (2.13 g, 14.5 mmol) was dissolved in dichloromethane (100 mL), pyridine was added (5.7 mL), and the reaction was cooled to 0° C. Methyl carbonochloridate (1.13 mL, 15.9 mmol) was added slowly and the reaction was allowed to stir at 0° C. for 1 h followed by 2 h at room temperature. The reaction was then washed with water (100 mL), HCl (0.05 N aq., 50 mL), saturated NaHCO₃ (100 mL), and brine (75 mL). The organic layer was separated, dried (Na₂SO₄), and concentrated to an oil. The oil was flashed via column chromatography (25% ethyl acetate in hexanes) to yield 0.92 g (31%) of the product as a clear oil. ¹H NMR (400 MHz, DMSO-d₆) δ ppm 3.73 (s, 3H) 4.11 (s, 2H) 5.23 (s, 2H) 7.46 (d, J=4.29 Hz, 2H) 7.36-7.49 (m, 2H).

### Step C: Methyl 3-(2-(2-(cyanomethyl)benzyl)phenyl)acrylate

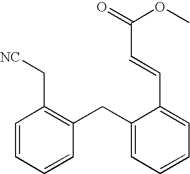

2-(3-methoxy-3-oxoprop-1-enyl)phenylboronic acid (227 mg, 1.1 mmol), potassium carbonate (304 mg, 2.2 mmol), [Pd(n³-C3H5)Cl](1.8 mg, 0.05 mmol), and 1,5-bis-(diphenylphosphino)pentane (4.4 mg, 0.10 mmol) were combined into a single vessel, and dimethylformamide (1 mL) was added. To the stirred mixture was added 2-(cyanomethyl)benzyl methyl carbonate (205 mg, 1.00 mmol) as a solution in dimethylformamide (1 mL). The reaction was heated to 80° C. and stirred for 16 h. The reaction was diluted with dichloromethane (15 mL) and washed with water (3×25 mL) and brine (20 mL). The organic layer was separated, dried (Na₂SO₄), and concentrated to an oil. Purification of the oil via column chromatography (20% ethyl acetate in hexanes) yielded 230 mg (79%) of the product as a white solid. ESI-MS: m/z 292.4 (M+H)⁺.

### Step D: Methyl 3-(2-(2-(2-acetamidoethyl)benzyl)phenyl)propanoate

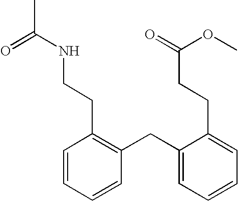

Methyl 3-(2-(2-(cyanomethyl)benzyl)phenyl)acrylate (230 mg, 0.79 mmol) was dissolved in acetic anhydride (1 mL) and an aliquot (˜1 mL) of well-mixed Ni-Raney suspension in Ac₂O [prepared by washing ˜3 mL of commercial Ni-Raney suspension in water with water (3×5 mL), ethanol (3×5 mL), acetic anhydride (3×5 mL), in that order, and suspending it in acetic anhydride (5 mL)] was added to the solution. The reaction mixture was charged with 60 psi hydrogen and heated to 50° C. with stirring for 3 h. The reaction was cooled to room temperature, diluted with dichloromethane (10 mL), and filtered through a plug of celite to remove the catalyst. The clear solution was then concentrated to afford the title compound as a white solid (244 mg, 91%). ESI-MS: m/z 340.4 (M+H)⁺.

### Step E: 3-(2-(2-(2-Acetamidoethyl)benzyl)phenyl)propanoic acid

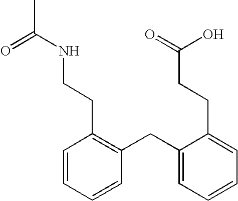

The title compound was prepared as described for Example 82. ESI-MS: m/z 326.4 (M+H)⁺.

### Step F: (R)—N-(2-(2-(3-(2-benzylpiperazin-1-yl)-3-oxopropyl)benzyl)phenethyl)acetamide

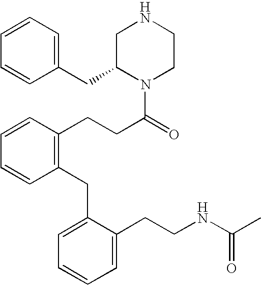

The title compound was prepared as described for Example 3. ¹H NMR (due to a ˜1:1 mixture of the tertiary amide rotamers, combined NMR spectral data for both rotamers are reported herein, leading to 2× hydrogen count) (400 MHz, DMSO-D6) δ, ppm: 1.65-1.81 (m, 7H), 2.33-3.41 (m, 30H), 3.83-3.89 (m, 3H), 4.03 (s, 1H), 4.34 (s, 1H), 4.55 (d, J=15.50 Hz, 1H), 4.85 (s, 1H), 6.75-6.90 (m, 6H), 7.04-7.32 (m, 10H), 8.01 (bs, 2H), 8.78 (bs, 2H), 8.99-9.09 (m, 2H). ESI-MS: m/z 484.5 (M+H)⁺.

### Example 85

### (R)-1-(2-(4-((1H-tetrazol-5-yl)methoxy)benzyl)piperazin-1-yl)-3-(2-phenoxyphenyl)propan-1-one

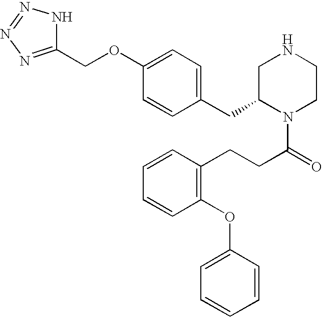

The title compound was prepared as described in Scheme 10. Specifically, (R)-2-(4-((4-benzyl-1-(3-(2-phenoxyphenyl)propanoyl)piperazin-2-yl)methyl)phenoxy)acetonitrile (200 mg, 0.366 mmol) was dissolved in dimethylformamide (1.3 mL). ammonium chloride (20.0 mg, 0.374 mmol) and sodium azide (24.1 mg, 0.371 mmol) were added and the reaction was stirred for 4 h at 100° C. The reaction was cooled to room temperature, diluted with dichloromethane (25 mL) and washed with water (20 mL) and brine (20 mL). The organic layer was dried (Na₂SO₄), and concentrated to an oil. The crude oil was dissolved in dioxane (5 mL) and ammonium formate (32 mg, 0.52 mmol) followed by the addition of Pd(OH)₂/C (10% w/w, 25 mg). The reaction mixture was stirred at 80° C. for 1 h in a sealed vial and cooled to room temperature. The mixture was filtered through celite and concentrated in vacuo. The residue was purified by HPLC (30-60% acetonitrile in water, TFA buffered) to afford the title compound as a white semisolid (12.0 mg, 6.5%). ESI-MS: m/z 499.4 (M+H)⁺.

### Example 86

### (R)-2-(4-((1-(3-(2-phenoxyphenyl)propanoyl)piperazin-2-yl)methyl)phenoxy)acetic acid

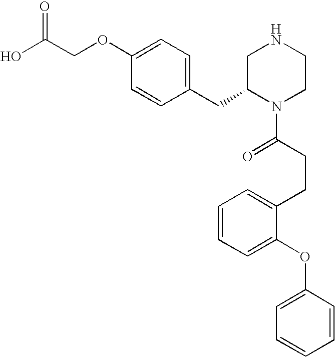

The title compound was prepared as described for Example 69 using bromoacetonitrile in place of methyl chloroacetate followed by deprotection as in Example 3 and saponification as in BDJ-462-100-1. ESI-MS: m/z 475.4 (M+H)⁺.

### Example 87

### (R)-1-(2-benzylpiperazin-1-yl)-3-(2-(2-(hydroxymethyl)phenoxy)phenyl)propan-1-one

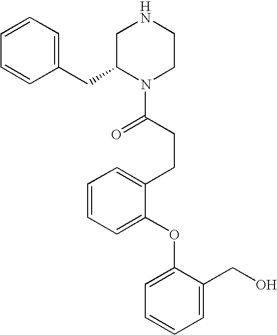

(R)-2-(2-(3-(2-Benzylpiperazin-1-yl)-3-oxopropyl)phenoxy)benzaldehyde (0.430 g, 0.813 mmol) was dissolved in tetrahydrofuran (5 mL) and methanol (5 mL). The reaction mixture was heated to 50° C. To the solution was added NaBH₄ (0.400 g, 10.6 mmol) portion-wise over the course of 3 h and the reaction was allowed to stir for 6 h at 50° C. The reaction mixture was diluted with ethyl acetate (35 mL) and poured slowly into NaHCO₃ (sat. aq., 50 mL). The resulting mixture was stirred at room temperature for 0.5 h, the organic layer was separated, dried (Na₂SO₄), and concentrated in vacuo to an oil. The residue was purified by HPLC (30-60% acetonitrile in water, TFA buffered) to afford the title compound as a white semisolid (27.0 mg, 7.7%). ESI-MS: m/z 431.4 (M+H)⁺.

### Example 88

### (R)—N-(2-(2-(3-(2-(4-hydroxybenzyl)piperazin-1-yl)-3-oxopropyl)benzyl)phenethyl)acetamide

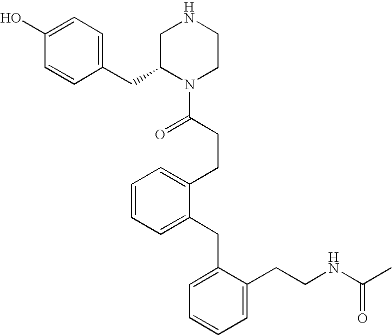

(R)-4-((4-benzylpiperazin-2-yl)methyl)phenol (prepared as described in Scheme 1) was coupled to 3-(2-(2-(2-acetamidoethyl)benzyl)phenyl)propanoic acid (prepared in Example 86, E) and the resulting compound was deprotected as described in procedure for Example 3, using DMF as a solvent for the coupling instead of dichloromethane. ESI-MS: m/z 500.4 (M+H)⁺.

### Example 89

### (R)-2-(4-((1-(3-(2-(2-(2-acetamidoethyl)benzyl)phenyl)propanoyl)piperazin-2-yl)methyl)phenoxy)acetamide

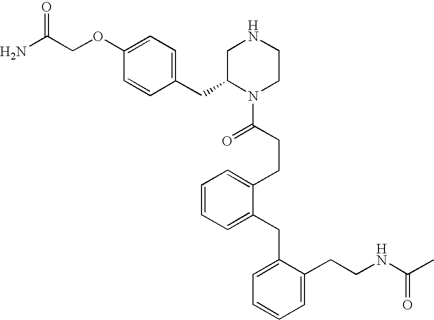

The title compound was prepared according to Scheme 12 (Steps 1-8a). Specifically, (R)-4-((4-benzylpiperazin-2-yl)methyl)phenol (prepared as described in Scheme 1) was coupled to 3-(2-(2-(2-acetamidoethyl)benzyl)phenyl)propanoic acid (prepared in Example 84, E) using procedure described in Example 72, A. The resulting (R)—N-(2-(2-(3-(4-benzyl-2-(4-hydroxybenzyl)piperazin-1-yl)-3-oxopropyl)benzyl)phenethyl)acetamide was used in the nucleophilic displacent of 2-chloroacetamide and the benzyl group was removed using procedure for Example 69. ESI-MS: m/z 557.4 (M+H)⁺.

### Example 90

### (R)-2-(4-((1-(3-(2-(2-(2-acetamidoethyl)benzyl)phenyl)propanoyl)piperazin-2-yl)methyl)phenoxy)acetic acid

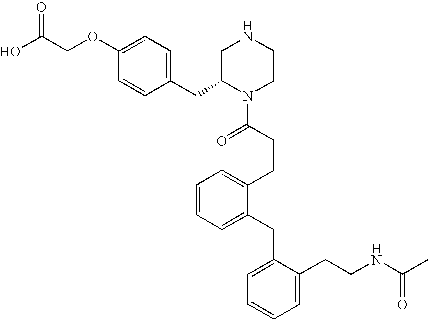

The title compound was prepared according to Scheme 12 (Steps 1-8a). Specifically, (R)-4-((4-benzylpiperazin-2-yl)methyl)phenol (prepared as described in Scheme 1) was coupled to 3-(2-(2-(2-acetamidoethyl)benzyl)phenyl)propanoic acid (prepared in Example 84, E) using procedure described in Example 72, A. The resulting (R)—N-(2-(2-(3-(4-benzyl-2-(4-hydroxybenzyl)piperazin-1-yl)-3-oxopropyl)benzyl)phenethyl)acetamide was used in the nucleophilic displacement of methyl 2-chloroacetate and the benzyl group was removed using procedure for Example 69. Next, the ester was saponified as described in Example 84. ESI-MS: m/z 558.4 (M+H)⁺.

### Example 91

### (R)-2-(4-((1-(3-(2-(2-(2-acetamidoethyl)benzyl)phenyl)propanoyl)piperazin-2-yl)methyl)phenoxy)-N-(methylsulfonyl)acetamide

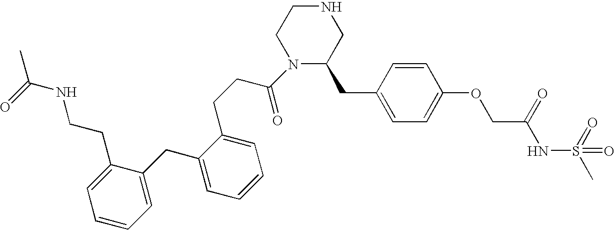

### Step A: (R)-methyl 2-(4-((1-(3-(2-(2-(2-acetamidoethyl)benzyl)phenyl)propanoyl)-4-benzylpiperazin-2-yl)methyl)phenoxy)acetate

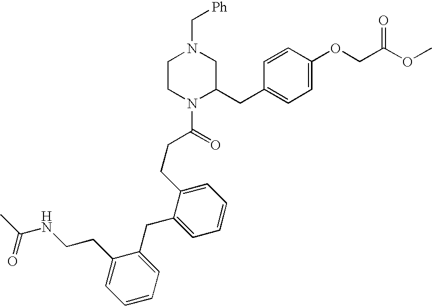

(R)—N-(2-(2-(3-(4-benzyl-2-(4-hydroxybenzyl)piperazin-1-yl)-3-oxopropyl)benzyl)phenethyl)acetamide (633 mg, 1.070 mmol) was dissolved in dimethylformamide (0.20 ml) and acetone (0.50 ml). Potassium carbonate (890.44 mg, 6.430 mmol) was added followed by the addition of methyl 2-chloroacetate (580 mg, 5.366 mmol). The reaction was heated to 70° C. and stirred for 15 hours. The reaction was then cooled to room temperature, filtered through celite, and concentrated to collect the product as a thick oil (676 mg, 95%). ESI-MS: m/z 662.5 (M+H)⁺.

### Step B: (R)-2-(4-((1-(3-(2-(2-(2-acetamidoethyl)benzyl)phenyl)propanoyl)-4-benzylpiperazin-2-yl)methyl)phenoxy)acetic acid

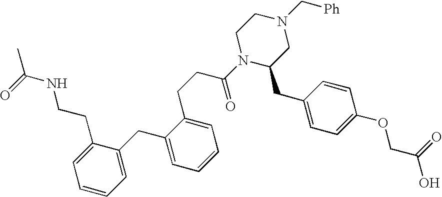

The title compound was prepared according to Scheme 12. Specifically (R)-methyl 2-(4-((1-(3-(2-(2-(2-acetamidoethyl)benzyl)phenyl)propanoyl)-4-benzylpiperazin-2-yl)methyl)phenoxy)acetate was saponified as described in Example 84. ESI-MS: m/z 648.5 (M+H)⁺.

### Step C: (R)-2-(4-((1-(3-(2-(2-(2-acetamidoethyl)benzyl)phenyl)propanoyl)-4-phenethyl piperazin-2-yl)methyl)phenoxy)-N-(methylsulfonyl)acetamide

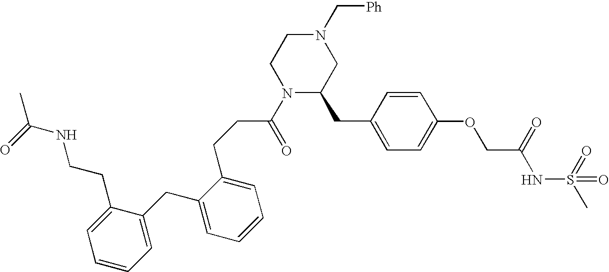

(R)-2-(4-((1-(3-(2-(2-(2-acetamidoethyl)benzyl)phenyl)propanoyl)-4-benzylpiperazin-2-yl)methyl)phenoxy)acetic acid (156 mg, 0.231 mmol) was dissolved in dimethylformamide (5 ml) under a blanket of nitrogen. To the solution was added methanesulfonamide (44 mg, 0.462 mmol), (2-(7-aza-1H-benzotriazole-1-yl)-1,1,3,3-tetramethyluronium hexafluorophosphate) (114 mg, 0.300 mmol) and Hunig's base (0.12 ml, 0.693 mmol). The reaction was allowed to stir at room temperature for 16 hours. The excess dimethylformamide was removed in vacuo. The residue was dissolved in ethyl acetate (30 ml) and washed with water (20 ml) followed by brine (20 ml). The organic layer was collected, dried with Na₂SO₄, and concentrated to a brown oil (158 mg, 94%) which was used crude without further purification. ESI-MS: m/z 725.5 (M+H)⁺.

### Step D: (R)-2-(4-((1-(3-(2-(2-(2-acetamidoethyl)benzyl)phenyl)propanoyl)piperazin-2-yl)methyl)phenoxy)-N-(methylsulfonyl)acetamide

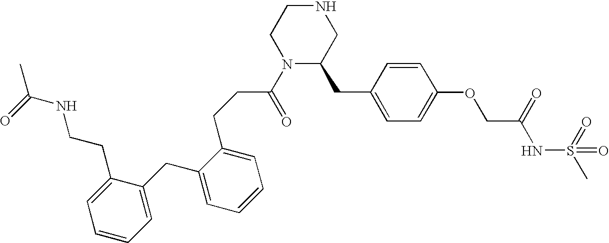

(R)-2-(4-((1-(3-(2-(2-(2-acetamidoethyl)benzyl)phenyl)propanoyl)-4-phenethyl piperazin-2-yl)methyl)phenoxy)-N-(methylsulfonyl)acetamide (85 mg, 0.12 mmol) was dissolved in dioxane (5 ml). To the solution was added palladium hydroxide (50 mg, 0.356 mmol) and ammonium formate (50 mg, 0.793 mmol). The reaction mixture was stirred in a sealed vessel at 80° C. for one hour. The reaction was then cooled to room temperature, filtered through celite, and concentrated to an oil. The residue was reconstituted in minimal dimethylsulfoxide and purified via HPLC to yield the TFA salt of the title compound as a white solid (24.9 mg, 28%).) (400 MHz, DMSO-D6) δ, ppm: 1.74-1.79 (m, 4H), 2.54-3.38 (m, 16H), 3.86 (d, J=12.95 Hz, 1H), 3.92 (s, 1H), 4.03 (s, 1H), 4.26 (s, 1H), 4.52 (d, J=12.95 Hz, 1H), 4.65 (s, 1H), 4.77 (s, 1H), 6.73-6.93 (m, 4H), 7.05-7.24 (m, 8H), 7.99 (bs, 1H), 8.72 (bs, 1H), 8.97 (d, J=41.71 Hz, 1H), 12.04 (s, 1H). ESI-MS: m/z 635.5 (M+H)⁺.

### Example 92

### (R)-Methyl 2-(4-((1-(3-(2-(2-(2-acetamidoethyl)benzyl)phenyl)propanoyl)piperazin-2-yl)methyl)phenoxy)acetate

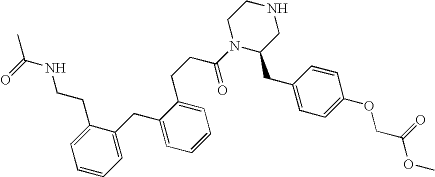

The title compound was prepared according to Scheme 12. Specifically, (R)-methyl 2-(4-((1-(3-(2-(2-(2-acetamidoethyl)benzyl)phenyl)propanoyl)-4-benzylpiperazin-2-yl)methyl)phenoxy)acetate was deprotected using the procedure described in Example 91, Step D. ESI-MS: m/z 572.5 (M+H)⁺.

### Example 93

### (R)-4-((1-(3-(2-(2-(2-acetamidoethyl)benzyl)phenyl)propanoyl)piperazin-2-yl)methyl)-N-(methylsulfonyl)benzamide

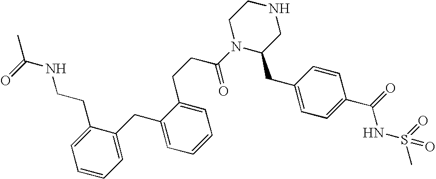

### Step A: (R)-4-((1-(3-(2-(2-(2-acetamidoethyl)benzyl)phenyl)propanoyl)-4-benzylpiperazin-2-yl)methyl)benzoic acid

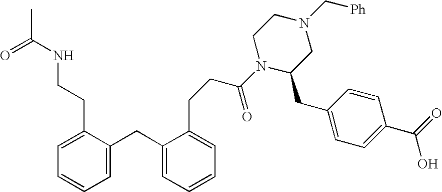

(R)—N-(2-(2-(3-(4-benzyl-2-(4-cyanobenzyl)piperazin-1-yl)-3-oxopropyl)benzyl)phenethyl)acetamide (255 mg, 0.426 mmol) was dissolved in methanol (5 ml) and an aqueous solution of potassium hydroxide (2M, 2.5 ml) was added. The reaction was stirred at 85° C. for 5 days. The reaction was then cooled to room temperature and neutralized with HCl (6N). The mixture was filtered and concentrated to an amorphous solid (257.4 mg, 98%) which was used in the next step without further purification. ESI-MS: m/z 618.5 (M+H)⁺.

### Step B: (R)-4-((1-(3-(2-(2-(2-acetamidoethyl)benzyl)phenyl)propanoyl)-4-benzylpiperazin-2-yl)methyl)-N-(methylsulfonyl)benzamide

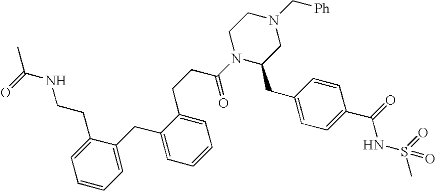

The title compound was prepared according to Scheme 12. Specifically, (R)-4-((1-(3-(2-(2-(2-acetamidoethyl)benzyl)phenyl)propanoyl)-4-benzylpiperazin-2-yl)methyl)benzoic acid was coupled using the procedure described in Example XX1, Step C. ESI-MS: m/z 695.3 (M+H)⁺.

### Step C: (R)-4-((1-(3-(2-(2-(2-acetamidoethyl)benzyl)phenyl)propanoyl)piperazin-2-yl)methyl)-N-(methylsulfonyl)benzamide

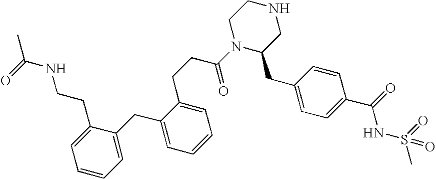

The title compound was prepared according to Scheme 12. Specifically, (R)-4-((1-(3-(2-(2-(2-acetamidoethyl)benzyl)phenyl)propanoyl)-4-benzylpiperazin-2-yl)methyl)-N-(methylsulfonyl)benzamide was deprotected using the procedure described in Example 91, Step D. ESI-MS: m/z 605.4 (M+H)⁺.

### Example 94

### (R)—N-(2-(2-(3-(2-(4-(1H-tetrazol-5-yl)benzyl)piperazin-1-yl)-3-oxopropyl)benzyl)phenethyl)acetamide

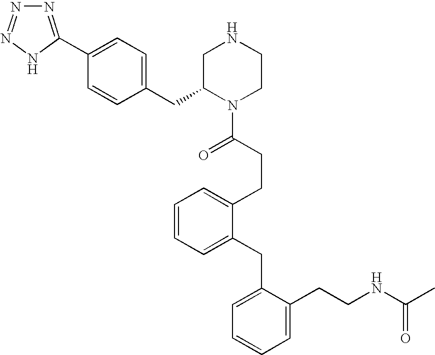

### Step A: (R)-tert-butyl 4-benzyl-2-(4-hydroxybenzyl)piperazine-1-carboxylate

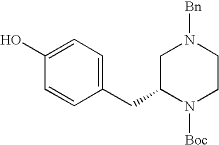

(R)-4-((4-benzylpiperazin-2-yl)methyl)phenol (prepared as described in Scheme 1, 2.66 g, 9.43 mmol) was suspended in MeOH/THF (1:1, 40 mL), cooled to 10° C. and treated with Boc₂O (2.20 g, 10.1 mmol). The reaction was stirred at 10° C. for 5 min and at room temperature for 1 h. It was concentrated in vacuo and the crude material was purified by flash column chromatography (90 g SiO₂, hexanes:ethyl acetate:dichloromethane 3:2:2, 700 mL) to afford the title compound as a white solid (2.86 g, 79%). ESI-MS: m/z 383.4 (M+H)⁺.

### Step B: (R)-tert-butyl 4-benzyl-2-(4-(trifluoromethylsulfonyloxy)benzyl)piperazine-1-carboxylate

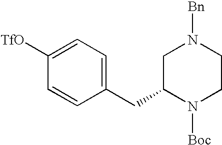

(R)-tert-butyl 4-benzyl-2-(4-hydroxybenzyl)piperazine-1-carboxylate (800 mg, 2.09 mmol) was treated with PhNTf₂ (768 mg, 2.15 mmol) and Et₃N (0.600 mL, 4.30 mmol) dichloromethane (5 mL) for 1.5 h. The reaction mixture was washed with water (5 mL), NaOH (aq. 1N, 5 mL), water (5 mL), dried (MgSO₄), filtered and concentrated in vacuo. Flash column chromatography on silica gel (80 g SiO₂, hexanes:ethyl acetate:dichloromethane 4:1:2, 700 mL) afforded the title compound as a white solid (1.01 g, 94%). ESI-MS: m/z 515.4 (M+H)⁺.

### Step C: (R)-tert-butyl 4-benzyl-2-(4-cyanobenzyl)piperazine-1-carboxylate

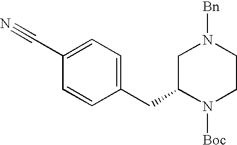

(R)-tert-butyl 4-benzyl-2-(4-(trifluoromethylsulfonyloxy)benzyl)piperazine-1-carboxylate (1.16 g, 2.25 mmol) was treated with Zn(CN)₂ (0.528 g, 4.50 mmol) and Pd(PPh₃)₄ (0.260 g, 0.225 mmol) in DMF (7 mL) under nitrogen atmosphere at 120° C. for 1.5 h. The reaction mixture was cooled to room temperature and directly loaded onto a column (90 g SiO₂, hexanes:ethyl acetate:dichloromethane 4:1:2, 700 mL) to afford the title compound as a white solid (0.515 g, 58%). ESI-MS: m/z 392.4 (M+H)⁺.

### Step D: (R)-4-((4-benzylpiperazin-2-yl)methyl)benzonitrile

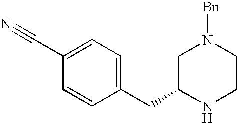

(R)-tert-butyl 4-benzyl-2-(4-cyanobenzyl)piperazine-1-carboxylate (507 mg, 1.29 mmol) was treated with trifluoroacetic acid (2 mL) in dichloromethane (4 mL) at room temperature for 1.5 h. The reaction mixture was concentrated in vacuo, the residue was cooled to 0 C and quenched with aq. NaOH (1M, ˜10 mL) to pH ˜13-14. The mixture was extracted with ethyl acetate (3×10 mL). The combined organic extracts were washed with brine (3 mL), dried (MgSO4), filtered and concentrated in vacuo to afford the title compound as a clear oil (0.350 g, 93%). ESI-MS: m/z 292.4 (M+H)⁺.

### Step E: (R)—N-(2-(2-(3-(4-benzyl-2-(4-cyanobenzyl)piperazin-1-yl)-3-oxopropyl)benzyl)phenethyl)acetamide

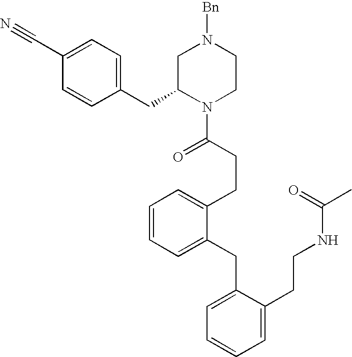

(R)-4-((4-benzylpiperazin-2-yl)methyl)benzonitrile (347 mg, 1.19 mmol) and 3-(2-(2-(2-acetamidoethyl)benzyl)phenyl)propanoic acid (prepared as described in Example 82, 387 mg, 1.19 mmol) were dissolved in DMF (3 mL). EDCI (274 mg, 1.43 mmol) and HOBt (219 mg, 1.43 mmol) were added and the reaction mixture was stirred at room temperature for 12 h. The reaction mixture was diluted with ethyl acetate (20 mL) and washed with water (3 mL), NaHCO₃ (sat. aq., 5 mL) and water again (5 mL). The combined aqueous layers were back-extracted with ethyl acetate (5 mL). The combined organic extracts were washed with brine (5 mL), dried (MgSO₄), filtered and concentrated in vacuo. The resulting crude oil was purified using flash column chromatography (40 g SiO₂, ethyl acetate:methanol 98:2, 500 mL) to afford the title compound as a clear foam (0.632, 89%). ESI-MS: m/z 599.5 (M+H)⁺.

### Step F: (R)—N-(2-(2-(3-(2-(4-(1H-tetrazol-5-yl)benzyl)piperazin-1-yl)-3-oxopropyl)benzyl)phenethyl)acetamide

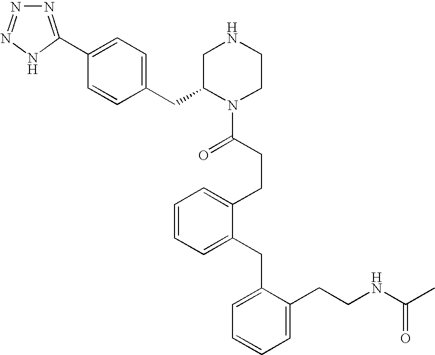

(R)—N-(2-(2-(3-(4-benzyl-2-(4-cyanobenzyl)piperazin-1-yl)-3-oxopropyl)benzyl)-phenethyl)acetamide (60.0 mg, 0.100 mg) was dissolved in DMF (0.3 mL) and triethylamine hydrochloride (16.0 mg, 0.116 mmol) was added. The reaction mixture was stirred for 2 min and NaN₃ (7.50 mg, 0.116 mmol) was added. The mixture was heated to 90° C. for 16 h and another portion of triethylamine hydrochloride (16.0 mg, 0.116 mmol) and NaN₃ (7.50 mg, 0.116 mmol) was added. The heating was continued for 6 h and the reaction mixture was cooled, diluted with water (3 mL), HCl (6 N, 0.3 mL) and extracted with ethyl acetate (10×2 mL) and dioxane (2×2 mL) ensuring dissolution of brown insoluble matter (contains the desired product). The combined organic layers were washed with brine (3 mL) and concentrated in vacuo and the brown poorly soluble residue was dried in high vacuum overnight and dissolved in dioxane (3 mL). Pd(OH)₂/C (20% w/w, 0.330 g) and ammonium formate (0.330 g) were added and the reaction mixture was heated at 90° C. in a closed vial (CAUTION: significant pressure build-up, use pressure-rated vessel) for 1.5 h. The reaction mixture was filtered through a small pad of celite, and the pad was washed well with dioxane (4×3 mL) and DMSO (2×1.5 mL). The combined filtrate and washes were concentrated in vacuo until only DMSO was left as a solvent and the resulting solution was used for HPLC purification (25-40% MeCN in water, 0.05% TFA buffered) to yield the title compound as a white solid (TFA salt, 28 mg, 42% yield for 2 steps). ESI-MS: m/z 552.4 (M+H)⁺.

### Example A

### Establishment of Renin Expressing Vector

A plasmid DNA to express human renin in HEK293 cells can be prepared as follows. PCR is carried out using human renal cDNA (Clontech Laboratories, Inc., Marathon Ready cDNA) as the template and using two synthetic DNAs (5′-AAGCTTATGGATGGATGGAGA-3′ (SEQ ID NO: 1) and 5′-GGATCCTCAGCGGGCCAAGGC-3′ (SEQ ID NO: 2)), and the obtained fragment is cloned using TOPO TA Cloning Kit (Invitrogen Corp.). The obtained fragment is subcloned into pcDNA3.1(+) that has been cleaved by HindIII and BamHI, to obtain a plasmid DNA for human preprorenin expression (pcDNA3.1(+)/hREN).

### Example B

### Expression of Preprorenin and Purification of Prorenin

The sequence of human wild-type Renin is known in the art; see, Imai, T. et al., Proc. Natl. Acad. Sci. USA 1983, 80, 7105-7409. It is noted that the fragment of the Renin protein useful for the assay comprises amino acid residues 67-406 of human Renin (active Renin). To prepare active Renin, a fragment longer than active Renin, a preprorenin (e.g., comprising residues 1-406), may be expressed and from which a prorenin (e.g., comprising residues 23-406) may be recovered. The prorenin may later be cleaved to obtain active Renin.

Expression of human preprorenin (residues 1-406) can be conducted using a FreeStyle 293 Expression System (Invitrogen Corp.), wherein the plasmid DNA for human prorenin expression (pcDNA3.1(+)/hREN) is used to conduct transient expression in FreeStyle 293-F cells. After transfection of the plasmid DNA, the cells are subjected to shaking at 37° C., 8% CO₂ and 125 rpm for 3 days.

The prorenin protein is then accumulated and purified by salting out. Powdered ammonium sulfate is added to the culture medium and dissolved to make a 40% saturation of the salt. The resulting precipitate can be collected by centrifugation and discarded. Ammonium sulfate is added to the remaining solution and dissolved to make an 80% saturation of salt. The resulting precipitate can be collected by, for example, centrifugation. The prorenin protein is recovered by dissolving the precipitate in buffer.

The concentrated liquid is subjected to gel filtration chromatography using, for example, HiLoad 16/60 Superdex 200 pg (Amersham Biosciences, Inc.) equilibrated with mM Tris-hydrochloric acid (pH 8.0) containing 0.15 M sodium chloride, at a flow rate of 1.4 ml/min, to obtain 3.6 mg of purified prorenin (residues 24-406).

### Example C

### Purification of Active Renin

To 3.6 mg of prorenin (residues 24-406, as prepared in Example A) dissolved in 5.2 ml of 0.1 M Tris-hydrochloric acid (pH 8.0), is added 12 g of trypsin (Roche Diagnostics Corp.), and the mixture is allowed to react at 28° C. for 55 minutes to carry out activation of Renin. After the reaction, 0.4 ml of immobilized trypsin inhibitor (Pierce Biotechnology, Inc.) is added to remove the trypsin used in the activation, by adsorption. The reaction liquid containing the active renin is concentrated using Vivaspin 20 (molecular weight of the fraction 10,000; Vivascience, Inc.), and diluted with 20 mM Tris-hydrochloric acid (pH 8.0). The diluted liquid is fed to a TSKgel DEAE-5 PW column (7.5 mm I.D.×75 mm, Tosoh Corp.) equilibrated with 20 mM Tris-hydrochloric acid (pH 8.0) at a flow rate of 1 ml/min to adsorb the active renin (residues 67-406). The column is washed with the buffer solution used for the equilibration, and then elution is carried out by means of a linear concentration gradient of sodium chloride from 0 M to 0.3 M, to obtain 1.5 mg of purified active renin (residues 67-406).

### Example D

### Assaying the In Vitro Enzymatic Activity of Renin Inhibitors

Solutions of test compounds in varying concentrations (<2 mM final concentration) are prepared in dimethyl sulfoxide (DMSO) and then diluted into assay buffer comprising 50 mM Hepes, 1 mM EDTA, 1 mM DTT, 0.1 mg/ml BSA, 0.01% Brij35, pH 7.4. Alternatively, the assay can be performed with a high BSA concentration, wherein the buffer contains an additional 2% BSA.

Recombinant human renin (3 nM final concentration) is added to the dilutions and pre-incubated with the compounds for 10 minutes at 37° C. As described in Examples A-C above, human Renin can be obtained by expressing preprorenin (residue 1-406) in mammalian cells, treating the prorenin (residues 24-406) contained in the culture supernatant with trypsin, and isolate the active form (residues 67-406). After pre-incubation, the reaction is initiated with 1 μM of substrate QXL520-γ-Abu-Ile-His-Pro-Phe-His-Leu-Val-Ile-His-Thr-Lys (HiLyteFluo488)-Arg-OH (Anaspec, San Jose, Calif.). The final DMSO in the assay is 5%. The total volume of the reaction mixture is 20 μL, which can be placed on Greiner 384-well small volume plates.

Renin activity may be determined via fluorescence (excitation λ=485 nm; emission λ=538 nm), e.g., on a Molecular Devices SPECTROmax GEMINI XPS. The fluorescence intensity is determined upon the addition of substrate and determined again after incubation at 37° C. for one hour. The fluorescence intensity of a blank (no inhibition) using vehicle alone is also determined. Renin activity is linearly proportional to the change in fluorescence observed (final−initial).

The percent inhibition of Renin at a given compound concentration is defined as:

100%×[1−(F_{compound}/F_{blank})]

where F_{compound} is the observed fluorescence at a given concentration of test compound and F_{blank} is the observed fluorescence in the presence of vehicle alone.

The pIC₅₀ value (negative log of the molar concentration of the compound that produces 50% inhibition) of a test compound is calculated by non-linear least squares curve fitting of the equation:

Percent Inhibition=100%/(1+(10^{-pIC50}/10^{log[I]}))

to percent inhibition versus compound concentration. The 50% inhibitory concentration (IC₅₀) of a test compound is calculated by raising 10 to the negative pIC₅₀ (10^{-pIC50}).

IC₅₀ values for selected compounds of the present invention are given in Table 1.

TABLE 1
IC₅₀ of Exemplified Compounds Against Renin

| EXAMPLE | IC₅₀ (μM) |
|---|---|
| 1 | 5-50 |
| 2 | 0.5-5 |
| 3 | 0.5-5 |
| 4 | 5-50 |
| 5 | >50 |
| 6 | 0.5-5 |
| 7 | <0.5 |
| 8 | 5-50 |
| 9 | 5-50 |
| 10 | >50 |
| 11 | 0.5-5 |
| 12 | 5-50 |
| 13 | 0.5-5 |
| 14 | 0.5-5 |
| 15 | 5-50 |
| 16 | 0.5-5 |
| 17 | 0.5-5 |
| 18 | 5-50 |
| 19 | 5-50 |
| 20 | 0.5-5 |
| 21 | 0.5-5 |
| 22 | <0.5 |
| 23 | <0.5 |
| 24 | <0.5 |
| 25 | <0.5 |
| 26 | <0.5 |
| 27 | <0.5 |
| 28 | <0.5 |
| 29 | <0.5 |
| 30 | 0.5-5 |
| 31 | <0.5 |
| 32 | 0.5-5 |
| 33 | 0.5-5 |
| 34 | 0.5-5 |
| 35 | 0.5-5 |
| 36 | 0.5-5 |
| 37 | 0.5-5 |
| 38 | 0.5-5 |
| 39 | <0.5 |
| 40 | <0.5 |
| 41 | <0.5 |
| 42 | 0.5-5 |
| 43 | <0.5 |
| 44 | 0.5-5 |
| 45 | 0.5-5 |
| 46 | <0.5 |
| 47 | 0.5-5 |
| 48 | 0.5-5 |
| 49 | <0.5 |
| 50 | <0.5 |
| 51 | 0.5-5 |
| 52 | 5-50 |
| 53 | <0.5 |
| 54 | 5-50 |
| 55 | 5-50 |
| 56 | <0.5 |
| 57 | >50 |
| 58 | 5-50 |
| 59 | >50 |
| 60 | 0.5-5 |
| 61 | <0.5 |
| 62 | 5-50 |
| 63 | 0.5-5 |
| 64 | 5-50 |
| 65 | 0.5-5 |
| 66 | <0.5 |
| 67 | <0.5 |
| 68 | <0.5 |
| 69 | 0.5-5 |
| 70 | <0.5 |
| 71 | 0.5-5 |
| 72 | 0.5-5 |
| 73 | 0.5-5 |
| 74 | 0.5-5 |
| 75 | 0.5-5 |
| 76 | 0.5-5 |
| 77 | 0.5-5 |
| 78 | <0.5 |
| 79 | <0.5 |
| 80 | 0.5-5 |
| 81 | <0.5 |
| 82 | 0.5-5 |
| 83 | <0.5 |
| 84 | <0.5 |
| 85 | <0.5 |
| 86 | 0.5-5 |
| 87 | <0.5 |
| 88 | <0.5 |
| 89 | <0.5 |
| 90 | <0.5 |
| 91 | <0.5 |
| 92 | <0.5 |
| 93 | <0.5 |
| 94 | <0.5 |

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

1-2. (canceled)

3. A compound consisting of the formula: 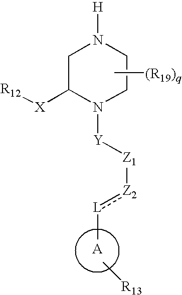
wherein:
q is selected from the group consisting of 0, 1, 2 and 3;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
L is absent or is a linker providing 1, 2 or 3 atom separation between Z₂ and R₂, wherein the atoms of the linker providing the separation are selected from the group consisting of carbon, oxygen, nitrogen, and sulfur;
X is —(CH₂)ₙ—, where n is selected from the group consisting of 1 and 2;
Y is selected from the group consisting of —CO— and —SO₂—;
Z₁ is selected from the group consisting of CR₆R₇, NR₈, O and S;
Z₂ is selected from the group consisting of CR₉R₁₀, NR₁₁, O and S, or Z₂ and one or more of the atoms of L providing the separation are taken together to form a 3, 4, 5, 6 or 7 membered ring;
R₆ and R₇ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, sulfonyl(C₁₋₃)alkyl, sulfinyl(C₁₋₃)alkyl, amino (C₁₋₁₀)alkyl and imino(C₁₋₃)alkyl, each substituted or unsubstituted, with the proviso that R₇ is absent when the atom to which it is bound forms part of a double bond, or R₆ and R₇ are taken together to form a ring;
R₈ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted;
R₉ and R₁₀ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together to form a ring, with the proviso that R₁₀ is absent when the atom to which it is bound forms part of a double bond;
R₁₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₁ is absent when the atom to which it is bound forms part of a double bond;
R₁₂ is a substituted or unsubstituted (C₄₋₇)aryl;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R₁₉ is selected from the group consisting of hydrogen, methyl and fluoromethyl,
with the proviso that Z₁ is not —O— when Y is —CO—, R₁ is hydrogen, X is —CH₂—, R₁₂ is phenyl, Z₂ is —CH₂—, ring A is phenyl and R₁₃ is hydrogen.

4. The compound according to claim 3 consisting of the formula: 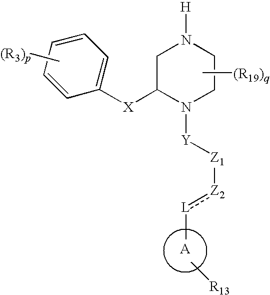
wherein:
p is selected from the group consisting of 0, 1, 2, 3, 4, and 5; and
R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted.

5. (canceled)

6. The compound according to claim 4 consisting of the formula: 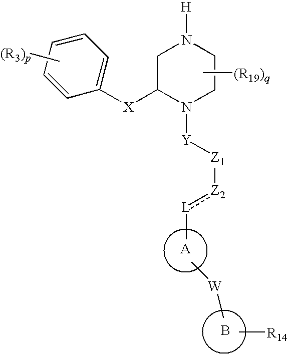
wherein:
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
W is absent or selected from the group consisting of CR₁₅R₁₆, NR₁₇, O and S;
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₁)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, with the proviso that R₁₆ is absent when the atom to which it is bound forms part of a double bond; and
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, with the proviso that R₁₇ is absent when the atom to which it is bound forms part of a double bond.

7-8. (canceled)

9. The compound according to claim 3, wherein X is —CH₂—.

10-11. (canceled)

12. The compound according to claim 3, wherein Z₁ is CR₆R₇.

13. The compound according to claim 3, wherein Z₂ is CR₉R₁₀.

14. The compound according to claim 12, wherein R₆ is hydrogen.

15. The compound according to claim 14, wherein R₇ is hydrogen.

16. The compound according to claim 3, wherein Z₁ is NR₈ where R₈ is selected from the group consisting of hydrogen and substituted or unsubstituted (C₁₋₃)alkyl.

17. The compound according to claim 13, wherein R₉ is selected from the group consisting of hydrogen, hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted.

18. The compound according to claim 17, wherein R₁₀ is hydrogen.

19. The compound according to claim 3, wherein Z₂ is NR₁₁ where R₁₁ is selected from the group consisting of hydrogen and substituted or unsubstituted (C₁₋₃)alkyl.

20-21. (canceled)

22. The compound according to claim 3, wherein R₃ is selected from the group consisting of 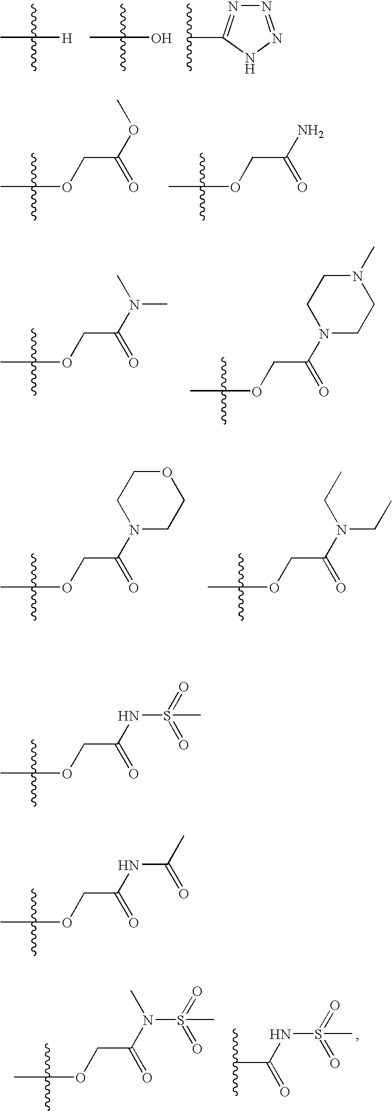
each substituted or unsubstituted.

23-25. (canceled)

26. The compound according to claim 3, wherein ring A is selected from the group consisting of phenyl and hetero(C₁₋₅)aryl, each substituted or unsubstituted.

27. The compound according to claim 26, wherein R₁₃ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted.

28. The compound according to claim 26, wherein R₁₃ is selected from the group consisting of 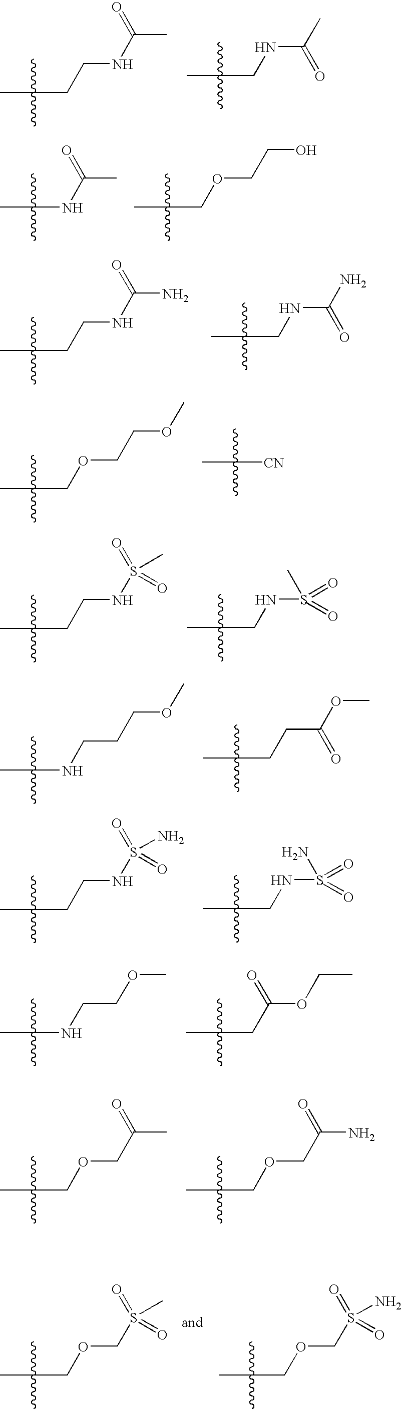
each substituted or unsubstituted.

29. The compound according to claim 6, wherein W is selected from the group consisting of CR₁₅R₁₆ where R₁₅ and R₁₆ are each independently selected from the group consisting of hydrogen, halo, hydroxyl and substituted or unsubstituted (C₁₋₃)alkyl, NH and O.

30. The compound according to claim 29, wherein R₁₅ is selected from the group consisting of hydrogen, hydroxyl, halo and substituted or unsubstituted (C₁₋₃)alkyl.

31. The compound according to claim 30, wherein R₁₆ is selected from the group consisting of hydrogen, hydroxyl, halo and substituted or unsubstituted (C₁₋₃)alkyl.

32. The compound according to claim 6, wherein W is —N(R₁₇)— where R₁₇ is selected from the group consisting of 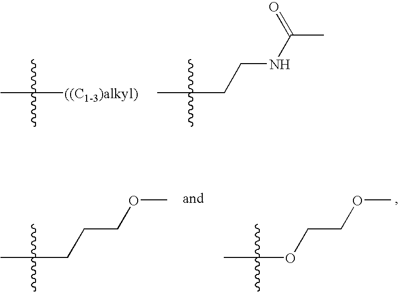
each substituted or unsubstituted.

33. (canceled)

34. The compound according to claim 6, wherein ring B is selected from the group consisting of phenyl and hetero(C₁₋₁₀)aryl, each substituted or unsubstituted.

35. The compound according to claim 6, wherein R₁₄ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, amido(C₁₋₁₀)alkyl, alkoxyalkoxy(C₁₋₁₀)alkyl, alkoxy(C₁₋₁₀)alkyl and alkoxyalkoxy, each substituted or unsubstituted.

36. The compound according to claim 6, wherein R₁₄ is selected from the group consisting of 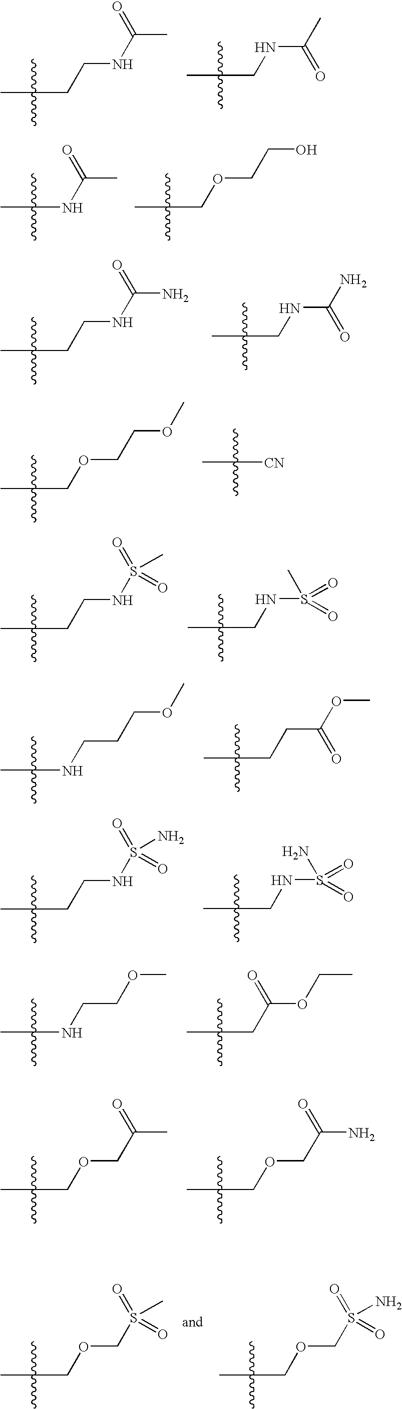
each substituted or unsubstituted.

37. The compound according to claim 3, wherein L is absent.

38. The compound according to claim 3, wherein Y is —CO—.

39. A compound selected from the group consisting of:
(R)-1-(2-benzylpiperazin-1-yl)-3,3-diphenylpropan-1-one;
(R)-1-(2-benzylpiperazin-1-yl)-2-(4′-fluorobiphenyl-2-yl)ethanone;
(R)-1-(2-benzylpiperazin-1-yl)-2-(2-bromophenoxy)ethanone;
(R)-1-(2-benzylpiperazin-1-yl)-4-phenylbutane-1,4-dione;
(R)-1-(2-benzylpiperazin-1-yl)-3-phenylpropan-1-one;
1-((R)-2-benzylpiperazin-1-yl)-2,3-diphenylpropan-1-one;
(R)-1-(2-benzylpiperazin-1-yl)-3-(2-phenoxyphenyl)propan-1-one;
(R)-1-(2-benzylpiperazin-1-yl)-2-(2-phenoxyphenyl)ethanone;
1-((R)-2-benzylpiperazin-1-yl)-2-(2′-methoxybiphenyl-2-yl)ethanone;
(R)—N-benzhydryl-2-benzylpiperazine-1-carboxamide;
1-((R)-2-benzylpiperazin-1-yl)-2-(2′-chlorobiphenyl-2-yl)ethanone;
(R)-2-benzyl-1-(phenethylsulfonyl)piperazine;
2-(3-((R)-2-benzylpiperazin-1-yl)-3-oxo-1-p-tolylpropyl)isoindolin-1-one;
(R)-1-(2-benzylpiperazin-1-yl)-2-(naphthalen-1-yloxy)ethanone;
1-((R)-2-benzylpiperazin-1-yl)-3-(furan-2-yl)-4-phenylbutan-1-one;
(R)-2-benzyl-1-(2,2-diphenylethylsulfonyl)piperazine;
(R)-1-((R)-2-benzylpiperazin-1-yl)-3-phenylbutan-1-one;
(S)-1-((R)-2-benzylpiperazin-1-yl)-3-phenylbutan-1-one;
(R)-1-(2-benzylpiperazin-1-yl)-4-phenylbutan-1-one;
(R)-1-(2-benzylpiperazin-1-yl)-3-(2-(4-chlorophenoxy)phenyl)propan-1-one;
1-((R)-2-benzylpiperazin-1-yl)-2-(2,2-dimethyl-4-phenyltetrahydro-2H-pyran-4-yl)ethanone;
(R)-2-(3-(2-benzylpiperazin-1-yl)-3-oxopropyl)phenyl benzoate;
(R)-2-(2-(3-(2-benzylpiperazin-1-yl)-3-oxopropyl)phenoxy)benzonitrile;
(R)-1-(2-benzylpiperazin-1-yl)-3-(2-(m-tolyloxy)phenyl)propan-1-one;
(R)—N-(2-(2-(3-(2-benzylpiperazin-1-yl)-3-oxopropyl)phenoxy)phenethyl)acetamide;
(R)—N-(3-(2-(3-(2-benzylpiperazin-1-yl)-3-oxopropyl)phenoxy)phenyl)acetamide;
(R)—N-(2-(2-(3-(2-benzylpiperazin-1-yl)-3-oxopropyl)phenoxy)benzyl)acetamide;
(R)-1-(2-benzylpiperazin-1-yl)-3-(2-(3-(dimethylamino)phenoxy)phenyl)propan-1-one;
(R)-1-(2-benzylpiperazin-1-yl)-3-(2-(3-ethoxyphenoxy)phenyl)propan-1-one;
(R)-3-(2-(3-(2-benzylpiperazin-1-yl)-3-oxopropyl)phenoxy)-N-ethylbenzamide;
(R)—N-((2-(2-(3-(2-benzylpiperazin-1-yl)-3-oxopropyl)phenoxy)pyridin-3-yl)methyl)acetamide;
(R)—N-((2-(2-(3-(2-benzylpiperazin-1-yl)-3-oxopropyl)phenoxy)-4,6-dimethylpyridin-3-yl)methyl)acetamide;
(R)—N-(2-(2-(3-(2-benzylpiperazin-1-yl)-3-oxopropyl)phenoxy)-5-methylbenzyl)acetamide;
(R)—N-(2-(2-(3-(2-benzylpiperazin-1-yl)-3-oxopropyl)phenoxy)-5-methoxybenzyl)acetamide;
(R)-1-(2-benzylpiperazin-1-yl)-3-(2-(p-tolyloxy)phenyl)propan-1-one;
(R)-1-(2-benzylpiperazin-1-yl)-3-(2-(4-methoxyphenoxy)phenyl)propan-1-one;
(R)—N-((2-(2-(3-(2-benzylpiperazin-1-yl)-3-oxopropyl)-4-methylphenoxy)-6-methylpyridin-3-yl)methyl)acetamide;
(R)—N-(2-(2-(3-(2-benzylpiperazin-1-yl)-3-oxopropyl)-4-methylphenoxy)-5-methylbenzyl)acetamide;
(R)—N-((2-(2-(3-(2-benzylpiperazin-1-yl)-3-oxopropyl)-5-methylphenoxy)-6-methylpyridin-3-yl)methyl)acetamide;
(R)—N-(2-(2-(3-(2-benzylpiperazin-1-yl)-3-oxopropyl)-5-methylphenoxy)-5-(trifluoromethyl)benzyl)acetamide;
(R)—N-(2-(2-(3-(2-benzylpiperazin-1-yl)-3-oxopropyl)-5-methylphenoxy)-5-methylbenzyl)acetamide;
(R)—N-(2-(2-(3-(2-benzylpiperazin-1-yl)-3-oxopropyl)-4-methylphenoxy)-5-methylphenethyl)acetamide;
(R)—N-(2-(2-(3-(2-benzylpiperazin-1-yl)-3-oxopropyl)-4-methylphenoxy)phenethyl)acetamide;
(R)—N-(2-(2-(3-(2-benzylpiperazin-1-yl)-3-oxopropyl)-5-methylphenoxy)-5-methylphenethyl)acetamide;
(R)—N-(2-(2-(3-(2-benzylpiperazin-1-yl)-3-oxopropyl)-5-methylphenoxy)phenethyl)acetamide;
(R)—N-(2-(2-(3-(2-benzylpiperazin-1-yl)-3-oxopropyl)-4-methylphenoxy)-5-(trifluoromethyl)benzyl)acetamide;
(R)—N-(3-(2-(2-(3-(2-benzylpiperazin-1-yl)-3-oxopropyl)phenoxy)phenyl)propyl)acetamide;
(R)—N-(2-(2-(3-(2-benzylpiperazin-1-yl)-3-oxopropyl)phenoxy)-5-methylphenethyl)acetamide;
2-(3-((R)-2-benzylpiperazin-1-yl)-3-oxopropyl)-2-phenylcyclohexanone;
(R)-3-(2-benzylphenyl)-1-(2-benzylpiperazin-1-yl)propan-1-one;
(R)-1-(2-benzylpiperazin-1-yl)-3-(4-(4-chlorophenyl)-2-methyloxazol-5-yl)propan-1-one;
(R)-3-(3-(2-benzylpiperazin-1-yl)-3-oxopropyl)-2H-benzo[b][1,4]oxazin-2-one;
(R)-1-(2-benzylpiperazin-1-yl)-3-(2-(4-chlorobenzyl)phenyl)propan-1-one;
(R)-1-(2-benzylpiperazin-1-yl)-2-(2-methoxyphenoxy)ethanone;
(R)-1-(2-benzylpiperazin-1-yl)-3-(diphenylamino)propan-1-one;
(R)-4-(3-(2-benzylpiperazin-1-yl)-3-oxopropyl)-2-methoxyphenyl acetate;
(R)-4-(2-benzylpiperazin-1-yl)-4-oxo-N,N-diphenylbutanamide;
1-((R)-2-benzylpiperazin-1-yl)-3-(2,2-dimethyltetrahydro-2H-pyran-4-yl)-3-phenylpropan-1-one;
(R)-2-(3-benzylphenoxy)-1-(2-benzylpiperazin-1-yl)ethanone;
(R)-1-(2-benzylpiperazin-1-yl)-3-(4,5-diphenyloxazol-2-yl)propan-1-one;
(R)—N-(1-(2-(2-benzylpiperazin-1-yl)-2-oxoethyl)cyclohexyl)benzamide;
(R)-2-(benzhydrylthio)-1-(2-benzylpiperazin-1-yl)ethanone;
(R)-3-(2-(2-(3-(2-benzylpiperazin-1-yl)-3-oxopropyl)phenoxy)phenyl)propanenitrile;
(R)-methyl 3-(2-(2-(3-(2-benzylpiperazin-1-yl)-3-oxopropyl)phenoxy)phenyl)propanoate;
(R)-1-(2-(4-hydroxybenzyl)piperazin-1-yl)-3-(2-phenoxyphenyl)propan-1-one;
(R)-methyl 2-(4-((1-(3-(2-phenoxyphenyl)propanoyl)piperazin-2-yl)methyl)phenoxy)acetate;
(R)-2-(4-((1-(3-(2-phenoxyphenyl)propanoyl)piperazin-2-yl)methyl)phenoxy)acetamide;
(R)—N,N-dimethyl-2-(4-((1-(3-(2-phenoxyphenyl)propanoyl)piperazin-2-yl)methyl)phenoxy)acetamide;
(R)-4-((1-(3-(2-phenoxyphenyl)propanoyl)piperazin-2-yl)methyl)phenyl 4-methylpiperazine-1-carboxylate;
(R)-4-((1-(3-(2-phenoxyphenyl)propanoyl)piperazin-2-yl)methyl)phenyl morpholine-4-carboxylate;
(R)-4-((1-(3-(2-phenoxyphenyl)propanoyl)piperazin-2-yl)methyl)phenyl diethylcarbamate;
(R)-1-(2-benzylpiperazin-1-yl)-3-(2-(3-hydroxypropoxy)phenyl)propan-1-one;
(R)-1-(2-benzylpiperazin-1-yl)-3-(2-(3-methoxypropoxy)phenyl)propan-1-one;
(R)-1-(2-benzylpiperazin-1-yl)-3-(2-(3-(2-methoxyethoxy)propoxy)phenyl)propan-1-one;
(R)-1-(2-benzylpiperazin-1-yl)-3-(2-(3-(hydroxymethyl)phenoxy)phenyl)propan-1-one;
(R)-3-(2-(3-acetylphenoxy)phenyl)-1-(2-benzylpiperazin-1-yl)propan-1-one;
(R)-3-(2-(3-(2-benzylpiperazin-1-yl)-3-oxopropyl)phenoxy)benzamide;
(R)—N-(2-(2-(3-(2-benzylpiperazin-1-yl)-3-oxopropyl)phenoxy)phenethyl)propionamide;
(R)—N-(2-(2-(3-(2-(4-hydroxybenzyl)piperazin-1-yl)-3-oxopropyl)phenoxy)phenethyl)acetamide;
(R)-4-((1-(3-(2-(2-(2-acetamidoethyl)phenoxy)phenyl)propanoyl)piperazin-2-yl)methyl)phenyl morpholine-4-carboxylate;
(R)-2-(4-((1-(3-(2-(2-(2-acetamidoethyl)phenoxy)phenyl)propanoyl)piperazin-2-yl)methyl)phenoxy)acetamide;
(R)-3-(2-(2-(3-(2-benzylpiperazin-1-yl)-3-oxopropyl)phenoxy)phenyl)propanoic acid;
(R)—N-(2-(2-(3-(2-benzylpiperazin-1-yl)-3-oxopropyl)benzyl)phenethyl)acetamide;
(R)-3-(2-(2-(3-(2-benzylpiperazin-1-yl)-3-oxopropyl)phenoxy)phenyl)propanamide;
(R)-1-(2-(4-((1H-tetrazol-5-yl)methoxy)benzyl)piperazin-1-yl)-3-(2-phenoxyphenyl)propan-1-one;
(R)-2-(4-((1-(3-(2-(2-(2-acetamidoethyl)benzyl)phenyl)propanoyl)piperazin-2-yl)methyl)phenoxy)acetamide;
(R)-2-(4-((1-(3-(2-(2-(2-acetamidoethyl)benzyl)phenyl)propanoyl)piperazin-2-yl)methyl)phenoxy)acetic acid;
(R)-1-(2-benzylpiperazin-1-yl)-3-(2-(2-(hydroxymethyl)phenoxy)phenyl)propan-1-one;
(R)—N-(2-(2-(3-(2-(4-hydroxybenzyl)piperazin-1-yl)-3-oxopropyl)benzyl)phenethyl)acetamide; and
(R)-2-(4-((1-(3-(2-phenoxyphenyl)propanoyl)piperazin-2-yl)methyl)phenoxy)acetic acid;
(R)-2-(4-((1-(3-(2-(2-(2-acetamidoethyl)benzyl)phenyl)propanoyl)piperazin-2-yl)methyl)phenoxy)-N-(methylsulfonyl)acetamide
(R)-Methyl 2-(4-((1-(3-(2-(2-(2-acetamidoethyl)benzyl)phenyl)propanoyl)piperazin-2-yl)methyl)phenoxy)acetate
(R)-4-((1-(3-(2-(2-(2-acetamidoethyl)benzyl)phenyl)propanoyl)piperazin-2-yl)methyl)-N-(methylsulfonyl)benzamide
(R)—N-(2-(2-(3-(2-(4-(1H-tetrazol-5-yl)benzyl)piperazin-1-yl)-3-oxopropyl)benzyl)phenethyl)acetamide

40-42. (canceled)

43. A pharmaceutical composition comprising as an active ingredient a compound according to any one of claim 3-4, 6, 9, 12-19, 22, 26-32, 34-39.

44-47. (canceled)

48. The pharmaceutical composition according to claim 43, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

49-54. (canceled)

55. A therapeutic method comprising administering a compound of claim 3 to a subject.

56. A method of inhibiting Renin comprising contacting Renin with a compound of claim 3.

57. A method of inhibiting Renin comprising causing a compound of claim 3 to be present in a subject in order to inhibit Renin in vivo.

58. A method of inhibiting Renin comprising administering a first compound to a subject that is converted in vivo to a second compound wherein the second compound inhibits Renin in vivo, the second compound being a compound according to claim 3.

59. A method of treating a disease state for which Renin possesses activity that contributes to the pathology and/or symptomology of the disease state, the method comprising causing a compound of claim 3 to be present in a subject in a therapeutically effective amount for the disease state.

60. A method of treating a disease state for which Renin possesses activity that contributes to the pathology and/or symptomology of the disease state, the method comprising administering a compound of claim 3 to a subject, wherein the compound is present in the subject in a therapeutically effective amount for the disease state.

61. A method of treating a disease state for which Renin possesses activity that contributes to the pathology and/or symptomology of the disease state, the method comprising administering a first compound to a subject that is converted in vivo to a second compound wherein the second compound inhibits Renin in vivo, the second compound being a compound according to claim 3.

62. The method according to claim 59, wherein the disease state is selected from the group consisting of cardiovascular disease, hypertension, congestive heart failure, myocardial infarction, renal protection, inflammation, neurological diseases and cancer.

63-100. (canceled)
